SEQUENCE LISTING (12 sequences)

SEQ ID NO: 1 - DNA, 4998 bp, Mus musculus
cgaggaagtgcggcgtgaagttgtggagctgagattgcccgccgctggggacccggagcccaggagcgccccttcccaggcggccccttccggcgccgcgcctgtgcctgccctcgccgcgccccgcgcccgcagcctggtccagcctgagccatggggccggagccgcagtgatcatcatggagctggcggcctggtgccgttgggggttcctcctcgccctcctgtcccccggagccgcgggtacccaagtgtgtaccggtaccgacatgaagttgcgactccctgccagtcctgagacccacctggacatgcttcgccacctctaccagggctgtcaggtggtgcagggcaatttggagcttacctacctgcccgccaatgccagcctctcattcctgcaggacatccaggaagtccagggatacatgctcatcgctcacaaccgagtgaaacacgtcccactgcagaggttgcgcatcgtgagagggactcagctctttgaggacaagtatgccctggctgtgctagacaaccgagaccctttggacaacgtcaccaccgccgccccaggcagaaccccagaagggctgcgggagctgcagcttcgaagtctcacagagatcttgaagggaggagttttgatccgtgggaaccctcagctctgctaccaggacatggttttgtggaaggatgtcctccgtaagaataaccagctggctcctgtcgacatggacaccaatcgttcccgggcctgtccaccttgtgccccaacctgcaaagacaatcactgttggggtgagagtcctgaagactgtcagatcttgactggcaccatctgtactagtggctgtgcccggtgcaagggccggctgcccactgactgttgccatgagcagtgtgctgcaggctgcacgggtcccaagcattctgactgcctggcctgcctccacttcaatcatagtggtatctgtgagctgcactgcccggccctcatcacctacaacacagacaccttcgagtccatgctcaaccctgagggtcgctacacctttggtgccagctgtgtgaccacctgcccctacaactacctctccacggaagtgggatcctgcactctggtctgtcccccgaacaaccaagaggtcacagctgaggacggaacacagcggtgtgagaaatgcagcaagccctgtgctggagtatgctatggtctgggcatggagcacctccgaggggcgagggccatcaccagtgacaatatccaggagtttgctggctgcaagaagatctttgggagcctggcatttttgccggagagctttgatgggaacccctcctccggcgttgccccactgaagccagagcatctccaagtgttcgaaaccctggaggagatcacaggttacctatacatttcagcatggccagagagcttccaagacctcagtgtcttccagaaccttcgggtcattcggggacggattctccatgatggtgcttactcattgacgttgcaaggcctggggattcactcactggggctacgctcactgcgggagctgggcagtggattggctctcattcaccgcaacacccatctctgctttgtaaacactgtaccttgggaccagctcttccggaacccgcaccaggccctactccacagtgggaaccggccagaagaggcatgtggtcttgagggcttggtctgtaactcactgtgtgcccgtgggcactgctgggggccagggcccacccagtgtgtcaactgcagtcagttcctccggggccaggagtgtgtggaggagtgccgagtatggaaggggctccccagggagtatgtgaggggcaagcactgtctgccatgccaccccgagtgtcagcctcaaaacagctcggagacctgctatggatcggaggctgaccagtgtgaggcttgtgcccactacaaggactcatcttcctgtgtggctcgctgccccagtggtgtgaagccagacctctcctacatgcctatctggaagtacccggatgaggagggcatatgtcagccatgccccatcaactgcacccactcatgtgtggacctggacgaacgaggctgcccagcagagcagagagccagcccagtgacattcatcattgcaactgtggtgggcgtcctgttgttcctgatcatagtggtggtcattggaatcctaatcaaacgaaggcgacagaagatccggaagtataccatgcgtaggctgctgcaggagaccgagctggtggagccgctgacgcccagtggagctgtgcccaaccaggctcagatgcggatcctaaaggagacagagctaaggaagctgaaggtgcttgggtcaggagccttcggcactgtctacaagggcatctggatcccagatggggagaacgtgaaaatccccgtggccatcaaggtgttgagggaaaacacatctcctaaagctaacaaagaaatcctagatgaagcgtacgtcatggctggtgtgggttctccatatgtgtcccgcctcctgggcatctgcctgacatccacagtgcagctggtgacacagcttatgccctatggctgccttctggaccatgtccgagaacaccgaggtcgcttaggctcccaggacctgctcaactggtgtgttcagattgccaaggggatgagctacctggaggaagttcggcttgttcacagggacctagctgcccgaaacgtgctagtcaagagtcccaaccacgtcaagattaccgacttcgggctggcacggctgctggacattgatgagactgaataccatgcagatgggggcaaggtgcccatcaagtggatggcattggaatctattctcagacgccggttcacccatcagagtgatgtgtggagctatggtgtgactgtgtgggagctgatgacctttggggccaaaccttacgatgggatcccagctcgggagatccctgatttgctggagaagggagaacgcctacctcagcctccaatctgcaccatcgacgtctacatgatcatggtcaaatgttggatgattgactccgaatgtcgcccgagattccgggagttggtatcagaattctcccgtatggcaagggacccccagcgctttgtggtcatccagaacgaggacttaggcccctccagccccatggacagcaccttctaccgttcactgctggaggatgatgacatgggggagctggtcgatgctgaagagtacctggtaccccagcagggattcttctccccagaccctgccctaggtactgggagcacagcccaccgcagacaccgcagctcgtcggccaggagtggcggtggtgagctgacactgggcctggagccctcggaagaagagccccccagatctccactggctccctccgaaggggctggctccgatgtgtttgatggtgacctggcagtgggggtaaccaaaggactgcagagcctctctccacatgacctcagccctctacagcggtacagtgaggatcccacattacctctgccccccgagactgatggctacgttgctcccctggcctgcagcccccagcccgagtatgtgaaccagccagaggttcggcctcagtctcccttgaccccagagggtcctccgcctcccatccgacctgctggtgctactctagaaagacccaagactctctctcctgggaaaaatggggttgtcaaagacgtttttgcctttgggggtgctgtggagaaccctgaatacttagcacccagagcaggcactgcctctcagccccacccttctcctgccttcagcccagcctttgacaacctctattactgggaccagaactcatcggagcagggtcctccaccaagtacctttgaagggacccccactgcagagaaccctgagtacctaggcctggatgtgccagtatgaggtcacatgtgcagacatcctctgtcttcagagtggggaaggaaggcctaacttgtggtctccatcgcccgccacaaagcagggagaaggtcctctggccacatgacatccagggcagccggctatgccaggaacgtgccctgaggaacctcgctcgatgcttcaatcctgagtggttaagagggccccgcctggccggaagagacagcacactgttcagccccagaggattacagaccctgactgccctgacagactgtagggtccagtgggtattccttacctggcctggctctcttggttctgaagactgagggaagctcagcctgcaagggaggaggccccaggtgaatatcctgggagcaggacaccccactaggactgaggcacgtgcatcccaagagggggacagcacttgcacccagactggtctttgtacagagtttattttgttctgtttttacttttgttttttgttttttttttaaagatgaaataaggatacagtgggagagtgggtgttatatgaaagtcggggggtgctgtcccctttctccatttgcaatgagatttgtaaaataactggaccccagcctatgtctgagagtggtcccgggccgggtcaaaccgtattgctcatctgacacacagctcctcctggagtgagtgtgtagagatcttccaaaagtttgagacaatttggctttgggcttgagggactggggagttaggattccttctgaaggccctttggcaacagggtcattctccgttggacacactcataccaaggctacccccagaatactccgttggacacactcattccaaggctacccccagaatgaagtcctgtcctcccagtgggagaggggagcttgtggagagcattgccatgtgacttgttttccttgccttagaaagaagtatccatccaggaaaaccccacccactaggtgttagtcccacccactaggtgttagcagggccagactgacctgtgtgccccccgcacaggctggacataaacacacgccagttgacacaa

SEQ ID NO: 2 - DNA, 4624 bp, Homo sapiens
ggaggaggtggaggaggagggctgcttgaggaagtataagaatgaagttgtgaagctgagattcccctccattgggaccggagaaaccaggggagccccccgggcagccgcgcgccccttcccacggggccctttactgcgccgcgcgcccggcccccacccctcgcagcaccccgcgccccgcgccctcccagccgggtccagccggagccatggggccggagccgcagtgagcaccatggagctggcggccttgtgccgctgggggctcctcctcgccctcttgccccccggagccgcgagcacccaagtgtgcaccggcacagacatgaagctgcggctccctgccagtcccgagacccacctggacatgctccgccacctctaccagggctgccaggtggtgcagggaaacctggaactcacctacctgcccaccaatgccagcctgtccttcctgcaggatatccaggaggtgcagggctacgtgctcatcgctcacaaccaagtgaggcaggtcccactgcagaggctgcggattgtgcgaggcacccagctctttgaggacaactatgccctggccgtgctagacaatggagacccgctgaacaataccacccctgtcacaggggcctccccaggaggcctgcgggagctgcagcttcgaagcctcacagagatcttgaaaggaggggtcttgatccagcggaacccccagctctgctaccaggacacgattttgtggaaggacatcttccacaagaacaaccagctggctctcacactgatagacaccaaccgctctcgggcctgccacccctgttctccgatgtgtaagggctcccgctgctggggagagagttctgaggattgtcagagcctgacgcgcactgtctgtgccggtggctgtgcccgctgcaaggggccactgcccactgactgctgccatgagcagtgtgctgccggctgcacgggccccaagcactctgactgcctggcctgcctccacttcaaccacagtggcatctgtgagctgcactgcccagccctggtcacctacaacacagacacgtttgagtccatgcccaatcccgagggccggtatacattcggcgccagctgtgtgactgcctgtccctacaactacctttctacggacgtgggatcctgcaccctcgtctgccccctgcacaaccaagaggtgacagcagaggatggaacacagcggtgtgagaagtgcagcaagccctgtgcccgagtgtgctatggtctgggcatggagcacttgcgagaggtgagggcagttaccagtgccaatatccaggagtttgctggctgcaagaagatctttgggagcctggcatttctgccggagagctttgatggggacccagcctccaacactgccccgctccagccagagcagctccaagtgtttgagactctggaagagatcacaggttacctatacatctcagcatggccggacagcctgcctgacctcagcgtcttccagaacctgcaagtaatccggggacgaattctgcacaatggcgcctactcgctgaccctgcaagggctgggcatcagctggctggggctgcgctcactgagggaactgggcagtggactggccctcatccaccataacacccacctctgcttcgtgcacacggtgccctgggaccagctctttcggaacccgcaccaagctctgctccacactgccaaccggccagaggacgagtgtgtgggcgagggcctggcctgccaccagctgtgcgcccgagggcactgctggggtccagggcccacccagtgtgtcaactgcagccagttccttcggggccaggagtgcgtggaggaatgccgagtactgcaggggctccccagggagtatgtgaatgccaggcactgtttgccgtgccaccctgagtgtcagccccagaatggctcagtgacctgttttggaccggaggctgaccagtgtgtggcctgtgcccactataaggaccctcccttctgcgtggcccgctgccccagcggtgtgaaacctgacctctcctacatgcccatctggaagtttccagatgaggagggcgcatgccagccttgccccatcaactgcacccactcctgtgtggacctggatgacaagggctgccccgccgagcagagagccagccctctgacgtccatcatctctgcggtggttggcattctgctggtcgtggtcttgggggtggtctttgggatcctcatcaagcgacggcagcagaagatccggaagtacacgatgcggagactgctgcaggaaacggagctggtggagccgctgacacctagcggagcgatgcccaaccaggcgcagatgcggatcctgaaagagacggagctgaggaaggtgaaggtgcttggatctggcgcttttggcacagtctacaagggcatctggatccctgatggggagaatgtgaaaattccagtggccatcaaagtgttgagggaaaacacatcccccaaagccaacaaagaaatcttagacgaagcatacgtgatggctggtgtgggctccccatatgtctcccgccttctgggcatctgcctgacatccacggtgcagctggtgacacagcttatgccctatggctgcctcttagaccatgtccgggaaaaccgcggacgcctgggctcccaggacctgctgaactggtgtatgcagattgccaaggggatgagctacctggaggatgtgcggctcgtacacagggacttggccgctcggaacgtgctggtcaagagtcccaaccatgtcaaaattacagacttcgggctggctcggctgctggacattgacgagacagagtaccatgcagatgggggcaaggtgcccatcaagtggatggcgctggagtccattctccgccggcggttcacccaccagagtgatgtgtggagttatggtgtgactgtgtgggagctgatgacttttggggccaaaccttacgatgggatcccagcccgggagatccctgacctgctggaaaagggggagcggctgccccagccccccatctgcaccattgatgtctacatgatcatggtcaaatgttggatgattgactctgaatgtcggccaagattccgggagttggtgtctgaattctcccgcatggccagggacccccagcgctttgtggtcatccagaatgaggacttgggcccagccagtcccttggacagcaccttctaccgctcactgctggaggacgatgacatgggggacctggtggatgctgaggagtatctggtaccccagcagggcttcttctgtccagaccctgccccgggcgctgggggcatggtccaccacaggcaccgcagctcatctaccaggagtggcggtggggacctgacactagggctggagccctctgaagaggaggcccccaggtctccactggcaccctccgaaggggctggctccgatgtatttgatggtgacctgggaatgggggcagccaaggggctgcaaagcctccccacacatgaccccagccctctacagcggtacagtgaggaccccacagtacccctgccctctgagactgatggctacgttgcccccctgacctgcagcccccagcctgaatatgtgaaccagccagatgttcggccccagcccccttcgccccgagagggccctctgcctgctgcccgacctgctggtgccactctggaaaggcccaagactctctccccagggaagaatggggtcgtcaaagacgtttttgcctttgggggtgccgtggagaaccccgagtacttgacaccccagggaggagctgcccctcagccccaccctcctcctgccttcagcccagccttcgacaacctctattactgggaccaggacccaccagagcggggggctccacccagcaccttcaaagggacacctacggcagagaacccagagtacctgggtctggacgtgccagtgtgaaccagaaggccaagtccgcagaagccctgatgtgtcctcagggagcagggaaggcctgacttctgctggcatcaagaggtgggagggccctccgaccacttccaggggaacctgccatgccaggaacctgtcctaaggaaccttccttcctgcttgagttcccagatggctggaaggggtccagcctcgttggaagaggaacagcactggggagtctttgtggattctgaggccctgcccaatgagactctagggtccagtggatgccacagcccagcttggccctttccttccagatcctgggtactgaaagccttagggaagctggcctgagaggggaagcggccctaagggagtgtctaagaacaaaagcgacccattcagagactgtccctgaaacctagtactgccccccatgaggaaggaacagcaatggtgtcagtatccaggctttgtacagagtgcttttctgtttagtttttactttttttgttttgtttttttaaagatgaaataaagacccagggggagaatgggtgttgtatggggaggcaagtgtggggggtccttctccacacccactttgtccatttgcaaatatattttggaaaacagcta

SEQ ID NO: 3 - DNA, 3724 bp, Homo sapiens
tctccctcggcgccgccgccgccgcccgcggggctgggacccgatgcggttagagccgcggagcctggaagagccccgagcgtttctgctttgggacaaccatacatctaattccttaaagtagttttatatgtaaaacttgcaaagaatcagaacaatgcctccacgaccatcatcaggtgaactgtggggcatccacttgatgcccccaagaatcctagtagaatgtttactaccaaatggaatgatagtgactttagaatgcctccgtgaggctacattaataaccataaagcatgaactatttaaagaagcaagaaaataccccctccatcaacttcttcaagatgaatcttcttacattttcgtaagtgttactcaagaagcagaaagggaagaattttttgatgaaacaagacgactttgtgaccttcggctttttcaaccctttttaaaagtaattgaaccagtaggcaaccgtgaagaaaagatcctcaatcgagaaattggttttgctatcggcatgccagtgtgtgaatttgatatggttaaagatccagaagtacaggacttccgaagaaatattctgaacgtttgtaaagaagctgtggatcttagggacctcaattcacctcatagtagagcaatgtatgtctatcctccaaatgtagaatcttcaccagaattgccaaagcacatatataataaattagataaagggcaaataatagtggtgatctgggtaatagtttctccaaataatgacaagcagaagtatactctgaaaatcaaccatgactgtgtaccagaacaagtaattgctgaagcaatcaggaaaaaaactcgaagtatgttgctatcctctgaacaactaaaactctgtgttttagaatatcagggcaagtatattttaaaagtgtgtggatgtgatgaatacttcctagaaaaatatcctctgagtcagtataagtatataagaagctgtataatgcttgggaggatgcccaatttgatgttgatggctaaagaaagcctttattctcaactgccaatggactgttttacaatgccatcttattccagacgcatttccacagctacaccatatatgaatggagaaacatctacaaaatccctttgggttataaatagtgcactcagaataaaaattctttgtgcaacctacgtgaatgtaaatattcgagacattgataagatctatgttcgaacaggtatctaccatggaggagaacccttatgtgacaatgtgaacactcaaagagtaccttgttccaatcccaggtggaatgaatggctgaattatgatatatacattcctgatcttcctcgtgctgctcgactttgcctttccatttgctctgttaaaggccgaaagggtgctaaagaggaacactgtccattggcatggggaaatataaacttgtttgattacacagacactctagtatctggaaaaatggctttgaatctttggccagtacctcatggattagaagatttgctgaaccctattggtgttactggatcaaatccaaataaagaaactccatgcttagagttggagtttgactggttcagcagtgtggtaaagttcccagatatgtcagtgattgaagagcatgccaattggtctgtatcccgagaagcaggatttagctattcccacgcaggactgagtaacagactagctagagacaatgaattaagggaaaatgacaaagaacagctcaaagcaatttctacacgagatcctctctctgaaatcactgagcaggagaaagattttctatggagtcacagacactattgtgtaactatccccgaaattctacccaaattgcttctgtctgttaaatggaattctagagatgaagtagcccagatgtattgcttggtaaaagattggcctccaatcaaacctgaacaggctatggaacttctggactgtaattacccagatcctatggttcgaggttttgctgttcggtgcttggaaaaatatttaacagatgacaaactttctcagtatttaattcagctagtacaggtcctaaaatatgaacaatatttggataacttgcttgtgagatttttactgaagaaagcattgactaatcaaaggattgggcactttttcttttggcatttaaaatctgagatgcacaataaaacagttagccagaggtttggcctgcttttggagtcctattgtcgtgcatgtgggatgtatttgaagcacctgaataggcaagtcgaggcaatggaaaagctcattaacttaactgacattctcaaacaggagaagaaggatgaaacacaaaaggtacagatgaagtttttagttgagcaaatgaggcgaccagatttcatggatgctctacagggctttctgtctcctctaaaccctgctcatcaactaggaaacctcaggcttgaagagtgtcgaattatgtcctctgcaaaaaggccactgtggttgaattgggagaacccagacatcatgtcagagttactgtttcagaacaatgagatcatctttaaaaatggggatgatttacggcaagatatgctaacacttcaaattattcgtattatggaaaatatctggcaaaatcaaggtcttgatcttcgaatgttaccttatggttgtctgtcaatcggtgactgtgtgggacttattgaggtggtgcgaaattctcacactattatgcaaattcagtgcaaaggcggcttgaaaggtgcactgcagttcaacagccacacactacatcagtggctcaaagacaagaacaaaggagaaatatatgatgcagccattgacctgtttacacgttcatgtgctggatactgtgtagctaccttcattttgggaattggagatcgtcacaatagtaacatcatggtgaaagacgatggacaactgtttcatatagattttggacactttttggatcacaagaagaaaaaatttggttataaacgagaacgtgtgccatttgttttgacacaggatttcttaatagtgattagtaaaggagcccaagaatgcacaaagacaagagaatttgagaggtttcaggagatgtgttacaaggcttatctagctattcgacagcatgccaatctcttcataaatcttttctcaatgatgcttggctctggaatgccagaactacaatcttttgatgacattgcatacattcgaaagaccctagccttagataaaactgagcaagaggctttggagtatttcatgaaacaaatgaatgatgcacatcatggtggctggacaacaaaaatggattggatcttccacacaattaaacagcatgcattgaactgaaaagataactgagaaaatgaaagctcactctggattccacactgcactgttaataactctcagcaggcaaagaccgattgcataggaattgcacaatccatgaacagcattagaatttacagcaagaacagaaataaaatactatataatttaaataatgtaaacgcaaacagggtttgatagcacttaaactagttcatttcaaaattaagctttagaataatgcgcaatttcatgttatgccttaagtccaaaaaggtaaactttgaagattgtttgtatctttttttaaaaaacaaaacaaaacaaaaatccccaaaatatatagaaatgatggagaaggaaaaaaaaaaaaaaaaa

SEQ ID NO: 4 - protein, 1068 aa, Homo sapiens
MPPRPSSGELWGIHLMPPRILVECLLPNGMIVTLECLREATLITIKHELFKEARKYPLHQLLQDESSYIFVSVTQEAEREEFFDETRRLCDLRLFQPFLKVIEPVGNREEKILNREIGFAIGMPVCEFDMVKDPEVQDFRRNILNVCKEAVDLRDLNSPHSRAMYVYPPNVESSPELPKHIYNKLDKGQIIVVIWVIVSPNNDKQKYTLKINHDCVPEQVIAEAIRKKTRSMLLSSEQLKLCVLEYQGKYILKVCGCDEYFLEKYPLSQYKYIRSCIMLGRMPNLMLMAKESLYSQLPMDCFTMPSYSRRISTATPYMNGETSTKSLWVINSALRIKILCATYVNVNIRDIDKIYVRTGIYHGGEPLCDNVNTQRVPCSNPRWNEWLNYDIYIPDLPRAARLCLSICSVKGRKGAKEEHCPLAWGNINLFDYTDTLVSGKMALNLWPVPHGLEDLLNPIGVTGSNPNKETPCLELEFDWFSSVVKFPDMSVIEEHANWSVSREAGFSYSHAGLSNRLARDNELRENDKEQLKAISTRDPLSEITEQEKDFLWSHRHYCVTIPEILPKLLLSVKWNSRDEVAQMYCLVKDWPPIKPEQAMELLDCNYPDPMVRGFAVRCLEKYLTDDKLSQYLIQLVQVLKYEQYLDNLLVRFLLKKALTNQRIGHFFFWHLKSEMHNKTVSQRFGLLLESYCRACGMYLKHLNRQVEAMEKLINLTDILKQEKKDETQKVQMKFLVEQMRRPDFMDALQGFLSPLNPAHQLGNLRLEECRIMSSAKRPLWLNWENPDIMSELLFQNNEIIFKNGDDLRQDMLTLQIIRIMENIWQNQGLDLRMLPYGCLSIGDCVGLIEVVRNSHTIMQIQCKGGLKGALQFNSHTLHQWLKDKNKGEIYDAAIDLFTRSCAGYCVATFILGIGDRHNSNIMVKDDGQLFHIDFGHFLDHKKKKFGYKRERVPFVLTQDFLIVISKGAQECTKTREFERFQEMCYKAYLAIRQHANLFINLFSMMLGSGMPELQSFDDIAYIRKTLALDKTEQEALEYFMKQMNDAHHGGWTTKMDWIFHTIKQHALN

SEQ ID NO: 5 - DNA, 5572 bp, Homo sapiens
cctcccctcgcccggcgcggtcccgtccgcctctcgctcgcctcccgcctcccctcggtcttccgaggcgcccgggctcccggcgcggcggcggagggggcgggcaggccggcgggcggtgatgtggcgggactctttatgcgctgcggcaggatacgcgctcggcgctgggacgcgactgcgctcagttctctcctctcggaagctgcagccatgatggaagtttgagagttgagccgctgtgaggcgaggccgggctcaggcgagggagatgagagacggcggcggccgcggcccggagcccctctcagcgcctgtgagcagccgcgggggcagcgccctcggggagccggccggcctgcggcggcggcagcggcggcgtttctcgcctcctcttcgtcttttctaaccgtgcagcctcttcctcggcttctcctgaaagggaaggtggaagccgtgggctcgggcgggagccggctgaggcgcggcggcggcggcggcacctcccgctcctggagcgggggggagaagcggcggcggcggcggccgcggcggctgcagctccagggagggggtctgagtcgcctgtcaccatttccagggctgggaacgccggagagttggtctctccccttctactgcctccaacacggcggcggcggcggcggcacatccagggacccgggccggttttaaacctcccgtccgccgccgccgcaccccccgtggcccgggctccggaggccgccggcggaggcagccgttcggaggattattcgtcttctccccattccgctgccgccgctgccaggcctctggctgctgaggagaagcaggcccagtcgctgcaaccatccagcagccgccgcagcagccattacccggctgcggtccagagccaagcggcggcagagcgaggggcatcagctaccgccaagtccagagccatttccatcctgcagaagaagccccgccaccagcagcttctgccatctctctcctcctttttcttcagccacaggctcccagacatgacagccatcatcaaagagatcgttagcagaaacaaaaggagatatcaagaggatggattcgacttagacttgacctatatttatccaaacattattgctatgggatttcctgcagaaagacttgaaggcgtatacaggaacaatattgatgatgtagtaaggtttttggattcaaagcataaaaaccattacaagatatacaatctttgtgctgaaagacattatgacaccgccaaatttaattgcagagttgcacaatatccttttgaagaccataacccaccacagctagaacttatcaaacccttttgtgaagatcttgaccaatggctaagtgaagatgacaatcatgttgcagcaattcactgtaaagctggaaagggacgaactggtgtaatgatatgtgcatatttattacatcggggcaaatttttaaaggcacaagaggccctagatttctatggggaagtaaggaccagagacaaaaagggagtaactattcccagtcagaggcgctatgtgtattattatagctacctgttaaagaatcatctggattatagaccagtggcactgttgtttcacaagatgatgtttgaaactattccaatgttcagtggcggaacttgcaatcctcagtttgtggtctgccagctaaaggtgaagatatattcctccaattcaggacccacacgacgggaagacaagttcatgtactttgagttccctcagccgttacctgtgtgtggtgatatcaaagtagagttcttccacaaacagaacaagatgctaaaaaaggacaaaatgtttcacttttgggtaaatacattcttcataccaggaccagaggaaacctcagaaaaagtagaaaatggaagtctatgtgatcaagaaatcgatagcatttgcagtatagagcgtgcagataatgacaaggaatatctagtacttactttaacaaaaaatgatcttgacaaagcaaataaagacaaagccaaccgatacttttctccaaattttaaggtgaagctgtacttcacaaaaacagtagaggagccgtcaaatccagaggctagcagttcaacttctgtaacaccagatgttagtgacaatgaacctgatcattatagatattctgacaccactgactctgatccagagaatgaaccttttgatgaagatcagcatacacaaattacaaaagtctgaatttttttttatcaagagggataaaacaccatgaaaataaacttgaataaactgaaaatggacctttttttttttaatggcaataggacattgtgtcagattaccagttataggaacaattctcttttcctgaccaatcttgttttaccctatacatccacagggttttgacacttgttgtccagttgaaaaaaggttgtgtagctgtgtcatgtatatacctttttgtgtcaaaaggacatttaaaattcaattaggattaataaagatggcactttcccgttttattccagttttataaaaagtggagacagactgatgtgtatacgtaggaattttttccttttgtgttctgtcaccaactgaagtggctaaagagctttgtgatatactggttcacatcctacccctttgcacttgtggcaacagataagtttgcagttggctaagagaggtttccgaagggttttgctacattctaatgcatgtattcgggttaggggaatggagggaatgctcagaaaggaaataattttatgctggactctggaccatataccatctccagctatttacacacacctttctttagcatgctacagttattaatctggacattcgaggaattggccgctgtcactgcttgttgtttgcgcatttttttttaaagcatattggtgctagaaaaggcagctaaaggaagtgaatctgtattggggtacaggaatgaaccttctgcaacatcttaagatccacaaatgaagggatataaaaataatgtcataggtaagaaacacagcaacaatgacttaaccatataaatgtggaggctatcaacaaagaatgggcttgaaacattataaaaattgacaatgatttattaaatatgttttctcaattgtaacgacttctccatctcctgtgtaatcaaggccagtgctaaaattcagatgctgttagtacctacatcagtcaacaacttacacttattttactagttttcaatcataatacctgctgtggatgcttcatgtgctgcctgcaagcttcttttttctcattaaatataaaatattttgtaatgctgcacagaaattttcaatttgagattctacagtaagcgttttttttctttgaagatttatgatgcacttattcaatagctgtcagccgttccacccttttgaccttacacattctattacaatgaattttgcagttttgcacattttttaaatgtcattaactgttagggaattttacttgaatactgaatacatataatgtttatattaaaaaggacatttgtgttaaaaaggaaattagagttgcagtaaactttcaatgctgcacacaaaaaaaagacatttgatttttcagtagaaattgtcctacatgtgctttattgatttgctattgaaagaatagggtttttttttttttttttttttttttttttaaatgtgcagtgttgaatcatttcttcatagtgctcccccgagttgggactagggcttcaatttcacttcttaaaaaaaatcatcatatatttgatatgcccagactgcatacgattttaagcggagtacaactactattgtaaagctaatgtgaagatattattaaaaaggtttttttttccagaaatttggtgtcttcaaattataccttcaccttgacatttgaatatccagccattttgtttcttaatggtataaaattccattttcaataacttattggtgctgaaattgttcactagctgtggtctgacctagttaatttacaaatacagattgaataggacctactagagcagcatttatagagtttgatggcaaatagattaggcagaacttcatctaaaatattcttagtaaataatgttgacacgttttccataccttgtcagtttcattcaacaatttttaaatttttaacaaagctcttaggatttacacatttatatttaaacattgatatatagagtattgattgattgctcataagttaaattggtaaagttagagacaactattctaacacctcaccattgaaatttatatgccaccttgtctttcataaaagctgaaaattgttacctaaaatgaaaatcaacttcatgttttgaagatagttataaatattgttctttgttacaatttcgggcaccgcatattaaaacgtaactttattgttccaatatgtaacatggagggccaggtcataaataatgacattataatgggcttttgcactgttattatttttcctttggaatgtgaaggtctgaatgagggttttgattttgaatgtttcaatgtttttgagaagccttgcttacattttatggtgtagtcattggaaatggaaaaatggcattatatatattatatatataaatatatattatacatactctccttactttatttcagttaccatccccatagaatttgacaagaattgctatgactgaaaggttttcgagtcctaattaaaactttatttatggcagtattcataattagcctgaaatgcattctgtaggtaatctctgagtttctggaatattttcttagactttttggatgtgcagcagcttacatgtctgaagttacttgaaggcatcacttttaagaaagcttacagttgggccctgtaccatcccaagtcctttgtagctcctcttgaacatgtttgccatacttttaaaagggtagttgaataaatagcatcaccattctttgctgtggcacaggttataaacttaagtggagtttaccggcagcatcaaatgtttcagctttaaaaaataaaagtagggtacaagtttaatgtttagttctagaaattttgtgcaatatgttcataacgatggctgtggttgccacaaagtgcctcgtttacctttaaatactgttaatgtgtcatgcatgcagatggaaggggtggaactgtgcactaaagtgggggctttaactgtagtatttggcagagttgccttctacctgccagttcaaaagttcaacctgttttcatatagaatatatatactaaaaaatttcagtctgttaaacagccttactctgattcagcctcttcagatactcttgtgctgtgcagcagtggctctgtgtgtaaatgctatgcactgaggatacacaaaaataccaatatgatgtgtacaggataatgcctcatcccaatcagatgtccatttgttattgtgtttgttaacaaccctttatctcttagtgttataaactccacttaaaactgattaaagtctcattcttgtcaaaaaaaaaaaaaaaaaaaaaaaaaaa

SEQ ID NO: 6 - protein, 403 aa, Homo sapiens [sequence omitted]

SEQ ID NO: 7 - DNA, 7709 bp, Homo sapiens
attttgaagagaggggtcccggggagctccctccaagatctagaggctccgcggccacccctgccgggtcctgccaagacttgctagaaggaacgagtcgcgtgccttagttagttggttcccgtcacaggaagaaacgcctttgcagtgggtttaattgcttctgggccgagcgaattccccgccgtacaactcagtggtgcggactttgcctcctgctaccctgttgctgcgccgagcggggtgggaaagtttctggagttgtcagtcgcgcagcccgtggccacctagacccgaggtgcgggcgcctgcgaagggcccccgcggggtggccggggccgccggggcatgcagcgcgggggcgcggctcggtgacgccgcgggcggggacccggcatccgggcaggctgcgcgcgggtgcggggcgagggcgccgcggggactgggacgcacggcccgcgcgcgggacacggccatggaggacgcgggagcagctggcccggggccggagcctgagcccgagcccgagccggagcccgagcccgcgccggagccggaaccggagcccaagccgggtgctggcacatccgaggcgttctcccgactctggaccgacgtgatgggtatcctggacggttcactgggaaacatcgatgacctggcgcagcagtatgcagattattacaacacctgtttctccgacgtgtgcgagaggatggaggagctgcggaaacggcgggtttcccaggacctggaagtggagaaacccgatgctagccccacgtcacttcagctgcggtcccagatcgaagagtcgcttggcttctgtagcgccgtgtcaaccccagaagtggaaagaaagaaccctcttcataaatcaaactcagaagacagctctgtaggaaaaggagactggaagaagaaaaataagtatttctggcagaacttccgaaagaaccagaaaggaataatgagacagacttcaaaaggagaagacgttggttatgttgccagtgaaataacgatgagcgatgaggagcggattcagctaatgatgatggtcaaagaaaagatgatcacaattgaggaagcacttgctaggctcaaggaatacgaggcccagcaccggcagtcggctgccctggaccctgctgactggccagatggttcttacccaacgtttgatggctcatcaaactgcaattcaagagaacaatcggatgatgagactgaggagtcggtgaagtttaagaggttacacaagctggtaaactccactcgcagagtcagaaagaaactaattagggtggaagaaatgaaaaaacccagcactgaaggtggggaggagcacgtgtttgagaattcgccggtcctggatgaacggtccgccctctactctggcgtgcacaagaagccccttttctttgatggctctcctgagaaacctcccgaagatgactcagactctctcaccacgtctccatcctccagcagcctggacacctggggggctggccggaagttggtcaaaaccttcagcaaaggagagagccggggcctgattaagccccccaagaagatggggacattcttctcctacccagaagaagaaaaggcccagaaagtgtcccgctccctcaccgagggggagatgaagaagggtctcgggtccctaagccacgggagaacctgcagttttggaggatttgacttgacgaatcgctctctgcacgttggcagtaataattctgacccaatgggtaaagaaggagactttgtgtacaaagaagtcatcaaatcacctactgcctctcgcatctctcttgggaaaaaggtgaaatcagtgaaagagacgatgagaaagagaatgtctaaaaaatacagcagctctgtctctgagcaggactcgggccttgatggaatgcctggctcccctccgccttcacagcccgaccccgaacacttggacaagcccaagctcaaggccgggggttctgtagaaagtcttcgcagttctctcagtgggcagagctccatgagcggtcaaacagtgagcaccactgattcctcaaccagcaaccgggaaagcgtcaagtcggaagatggggatgacgaagagccgccttaccgaggcccgttctgcgggcgtgccagggtgcacaccgacttcacccccagtccctatgacacagactcactcaagctcaagaaaggagatatcatcgatataatcagcaagccacccatggggacctggatgggcctgctgaacaacaaagtcggcacgttcaagttcatctacgtggacgtgctcagtgaagacgaggagaaacccaaacgccccaccaggaggcgtcggaaaggacgaccaccccagcccaagtctgtggaggatctcctggatcggattaacctaaaagagcacatgcccactttcctgttcaatggatatgaagatttggacacctttaagctgctggaggaggaagacttggatgagttaaatatcagggacccggaacacagagctgttctcttgacagcagtggagctgttacaagagtatgacagtaacagcgaccagtcaggatcccaggagaagctgctcgttgacagccagggcctgagtggatgctcaccccgagactcaggatgctacgaaagcagtgagaacctggaaaacggcaagactcggaaagctagcctcctatctgccaagtcatccaccgagcccagcttgaagtcttttagcagaaaccagttgggcaattacccaacattgcctttaatgaaatcaggggatgcactgaagcagggacaggaggagggcaggctgggtggtggccttgccccagacacgtccaagagctgtgacccacctggtgtgactggtttgaataaaaaccgaagaagcctcccagtttccatctgccggagctgtgagaccctggagggcccccagactgtggacacttggccccgatcccattccctggatgaccttcaagtggagcctggtgctgagcaagacgtgcctaccgaggtgacagaaccgccccctcagattgtacctgaagtgccacagaagacgaccgcctcttccacgaaggcccagcccctggagcaagactctgctgtcgacaatgcattgctactgacccaaagcaagagattttctgaacctcagaaattgacaactaagaaactggagggctcaatcgcagcctctggtcgcggcctgtcaccccctcagtgtttgcccagaaactatgatgctcagcctcctggagctaaacacggtttagcaaggacgcctctggagggccacagaaaaggacacgagtttgaaggaacacaccatcccctgggcaccaaagaaggggtagatgctgagcagagaatgcagcccaaaattccatcacagcctccacctgttcctgccaaaaagagcagagaacgccttgctaacggactccaccctgttcccatgggccccagtggggccctccccagtcccgatgcgccatgcctgccagtgaaaaggggcagccccgccagccccaccagccctagcgactgtcccccagcactggctcccaggcctctctcagggcaggcgcctggcagcccaccaagcacaaggccgcccccctggctctcagagctccccgagaacacaagcctccaggagcacggtgtgaagctgggcccggctttgaccaggaaggtctcctgtgcccggggagtggatctagaaacgctcactgaaaacaagctgcacgctgaaggcatcgatctcacggaggagccgtattctgataagcatggccgctgtgggattcctgaagccctggtgcagagatacgcagaggacttggatcagcccgagcgggacgtcgccgccaacatggaccagatccgggtgaagcagcttcggaagcagcaccgcatggcgattccaagtggtggactcacggaaatctgccgaaagcccgtctctcctgggtgcatttcgtctgtgtcagattggctcatttccatcggtctgcccatgtacgccggcaccctctccaccgcgggcttcagcacactgagccaagtgccttctctgtctcacacttgccttcaggaggccggcatcacagaggagagacacataagaaagctcctatctgcagccagactcttcaaactgccgccaggccctgaggccatgtagccaggcccggaatgggcctctctggacaagagccaccctttcactgtgcatatgatgctgatgcaattcctccatcatctctggacgtgcagaccagatccagaagaaaggcctggcgtgtggccaaacagcgtgaaaccttggcacaggactgaggatcctctcctccagaaaagccccctcgaggaaataaattagtgcggttctctttgacccccaaagacaagacaagcacttatttttattttcagaagacaaaagaaccaagatgccaactggctgcgaatgctctatctccagtctgtctctgtgtactggtagaggctgggaggagtagggggcagcctgttccatttctgatagtgcccttgctcttctgtctgtcatcttgcaggatgcccgagggccagatgggcttagctaggccaaagtaacagactcaagagttattgtacattactgaccacgctcatttgttcaaaagttagaacatctggctgcaccaggaaaaaaaaaaaaaaaaagtcctgttcttctttagataaacaagagacattttcataattgctttctagcaatcagcttttatttgccttaatataagcttttaagcagttatctaactagtgtccacaaccctgtaaccatacttccacatcttcagcttaggcagacatcgaacctctctgggatgtttccagcaaaagtgagcttttctaatcgtctcattgtaacatggcttattttgtagaggtattcatcagccacacacttcatgttggtttttggtttttaagctaactacaaatctagtaaaaagctatctgaaattcacaaatatcatgtgtgtgcgtgcgtgcgtgcgcgtgtgtgtctgtattcatagtgactgcttttggttttaaccagtttagtatcgttactgtgtggatcgtcgcgctgcagtattgacttggaatcctgaccatgtccatcccaaaattcagtcctcagttaacggatcatgtttgcaaaaggtcactgtgaggctgcatatttcagaaagatgtccttaataagggaagtcatgtataagatgttttctaaaagacttttcagtattacaactaatactattattatccttctttttttatttagataattcttttaatttaaacaaaggttcactatggaaccagacaaatctcattagccatgtgttaagtatttgctactttaaattgttttacaactgatttcagcacattctatccttttttttttttgaaatggagtttcgctcttgtcacccaggctggagtgcaatggcacgatcttggctcactgcaacctcagtctcccaggttcaagtgattctcctgccttagcctcccgagtagctgggattataggcacccaccaccacgcccagctaatttttgtattattagtagagacagggtttcaccatgttggccaggctggtctcaaactcaactcctgacctcaggtggtccacccgcctcagcctcccaaagtgctgggattacaggtgtgagccaccgcacctggcctctgtcctcttttagtctagtgtctggttttctagcaaacagtaaatttaaacaagtaaactattatggtttccattgcttacaaaatgattttcctttacattcttatcatgaacactattttaagcatcaaatgcaatcatctaaaatataaaggtcaatcatttataatagaaacaccttgaccacaagcccttgattgaacattttataatatttcatctacttattaaaacaaataatttcccttgggttggaggggaagtgatttcataaattaattagaaagccatctttagcatattgcttatgtctggatccatgtttctgaggaaaaagacattctcaggtgatgtatttttttcatgcattagtatgcatttttaaaaaataatgcatgtttctttaataattaattttcatcttctataagatgccatgtgaagaagttgtggaaatgtagaataaaaagctaaagctgccaaatttctgttgaactcttaaaaacagctcatgtttgtttgtcctctcgggttgtggcctagcctatttgcaatgtaatgaagctgcagggttcttgtatagctaaagcgttcaatgcatttcacgtgctgtggtggatgtgggtgctgtagacaggcttcttctcttcctgctctcaaaatacctcggcttgacatttggacagatcctgtcattgtttaagctgagcaaaaaaccacacaaaagttgtgtaagagatgagataacaaaggagcgagagaaatctcatgtgaatttccaagttttaattcgttctccatgaaggattttcatttcagtgaaagtcgcagcagaagagggaactttctggagtttttgagaatgccaaaccacatttttatcacacttctttggaaatcaatgcctttgcatagaaaatcaaattcagggaccacaaagaattttcagtgggaatgtctagtctgaggggtctgaggttgtttttactttattgtgttgtttaaatattttaaaaatatctttagcgtttggtcttttttttttctgtaaacatttaatttggtctgagaaaagctgaatgtttgggtgtgacgtttgactgaggtggattggggctgcctgtggacattagtgaacaggtggtaggcttcaggaatatccagttttaatcagttgcatttggtacagaattttgagtaatggtgaaaattgttgtctttggaaagcacaaaagaaacctggaaaggcagttcggctcaggtagctacacataacattgtgtatgattttcacttcaaagctgtctggaaggaaatgcagtcagctccagctagtactatttatgtacccagataactaagatattgtttcatggccttgccttagtcagaggcccttttctctgtcctgaacccccaggtatgggtgaaattggaaattactaatctattggaaatcagttcctgacatagtaaagtttgctttcataactgcagcaaaaaaggtcaacttgccaagtcactgctgccatgtgtgtactgtattattttcagaaaaaaatataatagtctgagtccaagttatcttgatttaaaattgatagagaaaagaaactgtcgagcaagttatataacaactaacaacattgcactttctgtatatgaaatcaatatttaaataacttatttttctccattgctgttcttaaaaacattgtaagtagctgtaatataccagtaccaatatgttcttgcaattgcttcagcccaagaaagctgtgtattgttttaaaaattgtaaaaattattgtgatgattcatttagcataaagagaggtggacggaagggttttcctatgtatcaaaacttgtctataattatgtcatctatgtacctagaaaaaagtaaataaatttcttcagttgaatatg

SEQ ID NO: 8 - protein, 1247 aa, Homo sapiens
MEDAGAAGPGPEPEPEPEPEPEPAPEPEPEPKPGAGTSEAFSRLWTDVMGILDGSLGNIDDLAQQYADYYNTCFSDVCERMEELRKRRVSQDLEVEKPDASPTSLQLRSQIEESLGFCSAVSTPEVERKNPLHKSNSEDSSVGKGDWKKKNKYFWQNFRKNQKGIMRQTSKGEDVGYVASEITMSDEERIQLMMMVKEKMITIEEALARLKEYEAQHRQSAALDPADWPDGSYPTFDGSSNCNSREQSDDETEESVKFKRLHKLVNSTRRVRKKLIRVEEMKKPSTEGGEEHVFENSPVLDERSALYSGVHKKPLFFDGSPEKPPEDDSDSLTTSPSSSSLDTWGAGRKLVKTFSKGESRGLIKPPKKMGTFFSYPEEEKAQKVSRSLTEGEMKKGLGSLSHGRTCSFGGFDLTNRSLHVGSNNSDPMGKEGDFVYKEVIKSPTASRISLGKKVKSVKETMRKRMSKKYSSSVSEQDSGLDGMPGSPPPSQPDPEHLDKPKLKAGGSVESLRSSLSGQSSMSGQTVSTTDSSTSNRESVKSEDGDDEEPPYRGPFCGRARVHTDFTPSPYDTDSLKLKKGDIIDIISKPPMGTWMGLLNNKVGTFKFIYVDVLSEDEEKPKRPTRRRRKGRPPQPKSVEDLLDRINLKEHMPTFLFNGYEDLDTFKLLEEEDLDELNIRDPEHRAVLLTAVELLQEYDSNSDQSGSQEKLLVDSQGLSGCSPRDSGCYESSENLENGKTRKASLLSAKSSTEPSLKSFSRNQLGNYPTLPLMKSGDALKQGQEEGRLGGGLAPDTSKSCDPPGVTGLNKNRRSLPVSICRSCETLEGPQTVDTWPRSHSLDDLQVEPGAEQDVPTEVTEPPPQIVPEVPQKTTASSTKAQPLEQDSAVDNALLLTQSKRFSEPQKLTTKKLEGSIAASGRGLSPPQCLPRNYDAQPPGAKHGLARTPLEGHRKGHEFEGTHHPLGTKEGVDAEQRMQPKIPSQPPPVPAKKSRERLANGLHPVPMGPSGALPSPDAPCLPVKRGSPASPTSPSDCPPALAPRPLSGQAPGSPPSTRPPPWLSELPENTSLQEHGVKLGPALTRKVSCARGVDLETLTENKLHAEGIDLTEEPYSDKHGRCGIPEALVQRYAEDLDQPERDVAANMDQIRVKQLRKQHRMAIPSGGLTEICRKPVSPGCISSVSDWLISIGLPMYAGTLSTAGFSTLSQVPSLSHTCLQEAGITEERHIRKLLSAARLFKLPPGPEAM

SEQ ID NO: 9 - DNA, 2878 bp, Homo sapiens
cggcaggaccgagcgcggcaggcggctggcccagcgcagccagcgcggcccgaaggacgggagcaggcggccgagcaccgagcgctgggcaccgggcaccgagcggcggcggcacgcgaggcccggccccgagcagcgcccccgcccgccgcggcctccagcccggccccgcccagcgccggcccgcggggatgcggagcggcgggcgccggaggccgcggcccggctaggcccgcgctcgcgcccggacgcggcggcccggggcttagggaaggccgagccagcctgggtcaaagaagtcaaaggggctgcctggaggaggcagcctgtcagctggtgcatcagaggctgtggccaggccagctgggctcggggagcgccagcctgagaggagcgcgtgagcgtcgcgggagcctcgggcaccatgagcgacgtggctattgtgaaggagggttggctgcacaaacgaggggagtacatcaagacctggcggccacgctacttcctcctcaagaatgatggcaccttcattggctacaaggagcggccgcaggatgtggaccaacgtgaggctcccctcaacaacttctctgtggcgcagtgccagctgatgaagacggagcggccccggcccaacaccttcatcatccgctgcctgcagtggaccactgtcatcgaacgcaccttccatgtggagactcctgaggagcgggaggagtggacaaccgccatccagactgtggctgacggcctcaagaagcaggaggaggaggagatggacttccggtcgggctcacccagtgacaactcaggggctgaagagatggaggtgtccctggccaagcccaagcaccgcgtgaccatgaacgagtttgagtacctgaagctgctgggcaagggcactttcggcaaggtgatcctggtgaaggagaaggccacaggccgctactacgccatgaagatcctcaagaaggaagtcatcgtggccaaggacgaggtggcccacacactcaccgagaaccgcgtcctgcagaactccaggcaccccttcctcacagccctgaagtactctttccagacccacgaccgcctctgctttgtcatggagtacgccaacgggggcgagctgttcttccacctgtcccgggagcgtgtgttctccgaggaccgggcccgcttctatggcgctgagattgtgtcagccctggactacctgcactcggagaagaacgtggtgtaccgggacctcaagctggagaacctcatgctggacaaggacgggcacattaagatcacagacttcgggctgtgcaaggaggggatcaaggacggtgccaccatgaagaccttttgcggcacacctgagtacctggcccccgaggtgctggaggacaatgactacggccgtgcagtggactggtgggggctgggcgtggtcatgtacgagatgatgtgcggtcgcctgcccttctacaaccaggaccatgagaagctttttgagctcatcctcatggaggagatccgcttcccgcgcacgcttggtcccgaggccaagtccttgctttcagggctgctcaagaaggaccccaagcagaggcttggcgggggctccgaggacgccaaggagatcatgcagcatcgcttctttgccggtatcgtgtggcagcacgtgtacgagaagaagctcagcccacccttcaagccccaggtcacgtcggagactgacaccaggtattttgatgaggagttcacggcccagatgatcaccatcacaccacctgaccaagatgacagcatggagtgtgtggacagcgagcgcaggccccacttcccccagttctcctactcggccagcggcacggcctgaggcggcggtggactgcgctggacgatagcttggagggatggagaggcggcctcgtgccatgatctgtatttaatggtttttatttctcgggtgcatttgagagaagccacgctgtcctctcgagcccagatggaaagacgtttttgtgctgtgggcagcaccctcccccgcagcggggtagggaagaaaactatcctgcgggttttaatttatttcatccagtttgttctccgggtgtggcctcagccctcagaacaatccgattcacgtagggaaatgttaaggacttctgcagctatgcgcaatgtggcattggggggccgggcaggtcctgcccatgtgtcccctcactctgtcagccagccgccctgggctgtctgtcaccagctatctgtcatctctctggggccctgggcctcagttcaacctggtggcaccagatgcaacctcactatggtatgctggccagcaccctctcctgggggtggcaggcacacagcagccccccagcactaaggccgtgtctctgaggacgtcatcggaggctgggcccctgggatgggaccagggatgggggatgggccagggtttacccagtgggacagaggagcaaggtttaaatttgttattgtgtattatgttgttcaaatgcattttgggggtttttaatctttgtgacaggaaagccctcccccttccccttctgtgtcacagttcttggtgactgtcccaccgggagcctccccctcagatgatctctccacggtagcacttgaccttttcgacgcttaacctttccgctgtcgccccaggccctccctgactccctgtgggggtggccatccctgggcccctccacgcctcctggccagacgctgccgctgccgctgcaccacggcgtttttttacaacattcaactttagtatttttactattataatataatatggaaccttccctccaaattcttcaataaaagttgcttttcaaaaaaaaaaaaaaaaaaaaaa

SEQ ID NO: 10 - protein, 480 aa, Homo sapiens [sequence omitted]

SEQ ID NO: 11 - DNA, 93190 bp, Homo sapiens
tttcgccctgttgcccaggttggtctcaaactgctgggctcaagtgatccacccacctcagcctcccaaagtgctgggattacaggcgtgagccaacacacctggctgaaaatagtcttttacgaatttcaaagctttgatttctccataagtgcactcctggtaacttgagaacattacacatttgtctcctttctgtggccagaattgttttgtatttcaagtatggaaaggagctttgccagaatcctcccgtgctaagtgtaagtagagcagttagactttaacatggttgtgtgatagtaatgacattcatatctgactctaggcaaggcatggctggaatgaaaaacattgactgttaagtgttttaaggtgaacttctcagatactagatctcttagctcacatagtggaattttgaggaaagtacagggtttgctcttcatctcctaagaaatgcaatgatgattctttaaagtactgatgggtcagggaggggagaaaaccattaaccgagaattgtataatcagctaaaatattttgcagaaatgaaggcaaaagtaaaatattctcaaatgaaggaaaatgaagggaatttgtcacaagcagactttctctaaaaagaatgttaaaattcttcaggataaagggaaattataccagaaggaaaaatggattttcaggaatgaagcaaaacataaatgtaaatggatacgtaaatataaaagattatttttcctcttgagttctatatgactgttgaaagcaaaaattataaaaacatctgatggggcttccatatatataaatgtaatacatatgccaactaatgacataaaggtcagggagtggggtaggagacagtggtaaatctacacggttgcaaggcttctttctacattttacttgaagtggtgtaatagtaatactaagtagaccgcaaaactttaggtatgtatattataatccatagaattaccaaaaaattaccaagaaatatattaaaactacaatatagaaattaaaatggaatattaaaaatttttcaaataatccacaagaaagcaggaaagtagaaacataaaaacaaaagtatagaggaaaaacagaaaacaaataataaaatggcaagcctaaatccatataattacattaaatgtaaattaaacacagcaattaaaacacagaaattgtcagattggaatttttaaaaagagatctaatacgtgttgtctacgagtaactcactttaaatataaaaatagatataaaaaagatgggaaaaaataccatggaaaaattaatcaaaaaaagctggactggctgtattaatatcagacaaagtagatttcagaacaaagatattactaggaataaagagagatagtacagattgattaaaaagagtcaatgtatcaaaacacaagagttctaaatgtgtatacatctaacaacaaagcttcaaaacacacaaagtaaaaatacacagaagtgaaatgagaaataaagaaatccacaattatattttgaaacatcaatgctaacaaaatcatcaacttggtaagaaactacattctgcccaacaatagcaaacaaaatattctttcataagtgcacatgggacattcaccaagatattctggggcaacacaaatcttaacttgaaaagaactgaaatcttaaaagtacgtgtgtgtatgtgtgtgtgggtgtatggtttttttttttttaagatggagtctcggtcaggcgtggtggctcacacctgtaatctcagcactttgggaggccaaggcgggcggatcatgaggtcaggagttcaagaccagcctgaccaatatggtgaaaccccgtctctactaaaaatacaaaaattagccgggcgtggtggcacgtgcctgtagtcccagctactcggaaggctgaggcaggagaatcacttgaacctgggaggcagaggttgcagtgagccaagatcgcgccactgcactccagcctgggtgacagagcgagactccatctcaaaaaaaaaaaaaaaaaaaaaaaaacagtgctacatggtaactcatatcgttatctcttggtgtgtttttattgtttgacttggcaattttatacatctaaagtatactgctacataatcaacttgactcttatttacaaaggacttcaaagcttagtagtgacttaagcgctattagagaatgtttcggtgcatatttgagtatcagttatgattttcaatggaaataaacccaaaaggcaagtaactgtcattataattctgattattagaatagaacaatagctacatactggacatttcacctcccgtctttagaacccttaggatgcaattatttaattttgaagtataccattttttgtttcatttacagcctgagaatcattatttttttaaaaccttctttcagctcctaatccttgagtgatgagtttggtcatatagatgcttatatcatacagatatttttatatcatatagatattttaaacactgaataacattgcaaaaacctttgaaagcaagatgacacctaagaccaatggcttacatataaggcaaacattatcaaccgtgaattgaatagtaaatgctactcctgtaccaatgaatggtgtcatgcattcaagtaccaggtatggctttttctgctatgacacacaacttcttaggggcagataatcacataacaaaaaacatattatgtaattagcattttcttattaaaaaataaattttaggctgggcatgtcggctgaagtctgtaatcccaacactttgggaggccgaggtagatgaatctcttgagcccaggagttggagaccagtctgggcaacgaagcaagaccctgtctttacaaaaaataaaacatttttaaaaatcacaaataggccaggtgcggtggcttacgcctgtaatcctagcactctgggaagtcgaggggggtggatcacctgaagtcagaagtttgagaccagcctgaccaacatggtgaaaccccgtctctactaagaaatacaaaaattagctggcatgttggctggtgcctgtaatcccagctactcgggaggctgaggcagaagaatcgcttgaacccgggaggcagaggctgcagtgagccgagactgcaccgctgaactccagcctgggctacagagtgagactctggctcaaaaaaataataataataaataaataataaattttataaggaaaaatatccctagtatatttttccccaccaaggtctacctattaatatctcatataactctgtgcttgaaacacaatttgggatatattgccttaaaatttactttgactagcaattcagtgttccttttttaaaaaaatctgtactctggagtaacagtgttctaaaactgttgaagaacattggttcgagaaaaacatttagaaacacaaacccctggaatgtgagatgaaaatccgagctaaagggagaaaaaggacgaaagaaagaaaacacagaaaagaaagaaatacagtcaagtgaaactacgaccacaaagaggaacagatccatctcatttaccattggggtatggacggacgtgcggtgttcgagaagactgttattggtcagagttttgctctagaggacaagtaggactgtaacatctctaggatgggtcacttcccaagccctctattacttaaaaattcaaggaggaaccgatgctggagtacttgtatctcagacttctaatcactgctcctacgcttttccaatattacaacaaaagaccagtagggggagaaaaacgcacagtaccgaacccttatcagtagtaatctcaaaagtcaacagattgatttactctcaagcaaacagacttctaaggtacgcagcaccaagacactaccttgaatcaaatctatagcctatatgacatttctgaagtctctgttggcattacgcgaaaaatcccccacgtcttctgaatagttagaattgaatcctacaagctgcttcgaatcagaattcgatttaaaaaaaaaaatgagggcatagcaaaaggtctccacgaagtgagtcaaaggactgcagagggctgtgacagtgcattccgccttcgggatggtatacaacttaaaccatgtcggcagaagaacgcacagcaacgctttgtaaaaagcattctttctattatagaatccataaccacgctggttagccactgacagcggcggttagccaccgcacctcctctcacccccgaactaatctcgtttcctctatgggtgtaaaagtgaaataacccacttgctcccaatattcctttctatatctctaccccagctcgcctgctgctcgtagaaacaaatatactacacgtacgctgtcctaggatgacacaacaccctcactactgcagaagacggatcattaaacaaacgtcagaagagcagccccaactgtacataaacttcgggcggaaaagcaagacgcaggcgcagtagcacatattgttaccctatttgcccactccctgctcctcctcgcctcaatttcgcttccgcttctttgcgcatctgcttccgggggattgtaggctctgcccctcctcagctcttaccctcttctgccggaggagggggggggccgagggggtggggaagagttcgttgtttgtttacacgatgtgagcggaaaaagagaccaataaagtttattctggaaacaaaaggaaaaaaaaacaggggcgacggagaaaggagtcgggggcgggggcgtgtggcgggggctagcgaggagagggagcgagaagtagaaagcggcagttccggtgccgccgctgcggccgctgaggtgtcgggctgctgctgccgcggccgctgggactggggctggggccgccggcgaggcagggctcgggcccggccgggcagctccggagcggcgggggagaggggccgggaggcgggggccgtgccgcccgctctcctctccctcggcgccgccgccgccgcccgcggggctgggacccgatgcggttagagccgcggagcctggaagagccccgagcgtgagtagagcgcggactggccggtagcgggtgcggtgggaatggggaccgggtgggtgccggagactcccgggcgcgcccgccgtgttgggcggaggctacgggctggtcgtctcccagcgtcggcccgcggcggagttcgcctgcgatcgccgcaccctctccctcgcctgcctctggcctctcctagctgcagaggcgagggctcgccgctccctctcctttctctctggctgccgcctcgctcttcctttgcttctactcccagttttggggacggggcggctggaggttgaggaagtgcgcccagggccgccgccccggccgcggtggctctgtctcccgcgccctgcacgcccacccagcccgcgggtggaggggctgccgcgaggactgcggctcaagggacgcgggcgcgaaaccgcgagcttttcttgaggggctgtcacgcccgaggactccggccccgccagcctggcggccctgcgtttgcctcttactgggtttaaatcgcccggcaggggtatggaaggaacaccgctcccctcacccccgccagctcccctcccacccttcctttcgaggttgttttttccttttagctgagacaaacgcttagatctttcattcatagacctgagacactgactcatgtgacttgtctctttactaagtgtattatgtgatgcctgttccatggctcttaagtggctcggaaaggtagggtagggcccgggattgggctatgtaaacaccagacgttcagccggcgtgtttttgacctcggctcgaggggtttattttaggatgctaaaggaatagtgatggtggccaaccgtgttaccagacttgggaatcgaaagctatttttttatgtcaataattgagactgtttacatggttaaataaagggtagggtaaagggagcgcaacaagagggtctttctagaaaacagtctgcatattctttaatgaattagagttgtgatttcatcttggtgaagcttcctcattccagcactgcgcctttgtgcacatgcaggacctgggttttcggaacgatgttttgaatcatagtttgtgtggccggggagaaccccttagaatacttgggtaggtaaattccaggttatctcaggggattatttctactgcgacttttgaaccactgagggcaatattggtcatatgttatcctgcttagaaattgaatttttctgagtatttgcttacattgcaccgtacaaaatttgtttatgtgctggattgcgtgttttaagaaaacttctggaaaatactcaaaataccgtgttgctaagttagtttggataaatcaaaacactttgctttcaaagcaactacagtcttaacaatttaacgtgtctttgtattcaggttttaagtttttggtgttttttttttttccttttaactgaagcactgaggtccgattgacagcatagatctccatctaaacatcacatgttctggaaatttttaattatcagattaactgttctgcagtgtcatcagccaaaattatctgaagaggactgtgtgtgtttacgtgtgtgtgtgtgtgtgtgtgtgtgtgtgtgttggtattttgcttaagttttttcatctcacattttgggacaaaataaaacttcatagcttttttcaaaagtttcttgtgctttttcaaactagagtactttcctattaaaatcacaaaatagtttatttaaaaatgatagcatatcgtaataatagcatcatgggggaaataacaagaaagtaaaggaatattaatattgcatgattgtgcgtataaagttttaaagacattcctgtcagttttaaataattaaaaatgaaacacctgtaaatatagaagagtgtttttatttaaggttaaaaaaatttaactaaattggttatagcattaaagagatgttactaaatacaaatattgtacttaaactgaaaaatggcttcatggaagtgtaaattgtcatgggccttgatatatttaaaagggagactaaaagtgcagtggttttgcttgctctgtgcgtggaacagcctttctggtggcagagcttccttcctgtcaggataaggtcctctccatcccaaatcagtatccccttaaaaaaccaaagaaaaaacccaaacttataaataatgcacaacataaagtagactgtaataactttttctctaaaccagtttactttgtgtgccgcttcttataagtaccctttactgaataaagagttttaaacagttacgattaccatttaaagggcacttgctctgttgatctgtttatttgaagagaggtaacttaatcccatttgatctcttgagcagctacagtagcattaaaaggatcagttctctattttgttattttatatcttgaattccaagataggggtgattctttaaatgaagtgtttcttaagcattataaaatatttcatctccagtgaaagtatttacaatcttaaaaattattcagctttagaagtatatgtcttagaatatcatggtttacattttattagcacagttatcaaatagtttttcttaagtattaccttgagctatattaacagtgccgtagacagagtggtattactatccctaagaaatggtattctaatctgaagtcagttggcaggtgacagactcattgacaaaaccaacattttccgactcctttcactgtatcatgctgcgtctaatctccagcctgttttttaatagaactgaagaaattagggcccaaggaggcttaattactgaagatctccatttcctatatttgcagtaatatctttatttaatttcctttacatcatgaacagtggttttcaatcctggctacacattagaataatctagggagctttttaaaagtactctggtctcatccccagagatgctcatttaattggtctgagatggatcccagttatctgtgttttgttttgtttgttttgaagttcaccaggaaatgctaatagaaaaggatgtaaagccctggctgtcaaggacctgtatatttggtccttgcttctttctctaggtttatttctcctccctactcctgcagctcccttggtcttttcatactctcctgtgcaaatacagtttgtacttcctgagacatttgaacactccattcttctttaactggttgactcctactcatctttcacatcacctcctttgaaaagtctagcttgagttaggtatccctgcttcctgttcccatagcatcgtgtactcccactgtcatagcattaacctcactgcattgtaattgcctatttctttgtccatatcctctaccaggctgtaaattccatgaggatagggactcctatcttatatactgtacagtagtattcttattgccaagcacagtagcctgtacattacagcacttgactaaacaaataaatatagtagagacagacttctttaaaacatgtagtgattcataagcaccagtttttattcttgccaatttcatttaatatttttggtcactaaaaatgtacaaccctgaaattgccacacatacgaatttcatttggggacagttgagatgttataaattgtatagagtggcattccacctcagacatatacacacacacgcaaaaaatgaagattattatcatatatagtactgcactggaaataaggagacttgaattcttacccctgtttcaccacaaacatttgggcacgtctcaatctctttgggtgtcatgtataatacagagggattgtacgaggtgctctacatggttctcagtgatctagccttaaaattctgtgagtatcaacagtattggctttggaatttgaggcaccatggttcaaaacctagttcttaaacttactggcaggattactttggacagattttcttatctgtaatgggggtgattactattcaacagagttgttgaattaaatgagataatgtagatccaagagagttcttgccctggatcaggtgcttaataaatgttcatttcctttctccttaggaagtgttagatctcatgagtggacttatctgagataacagaaggacacagtttatgtaataaatcctattttccactttgctgactgttagcactgcaggtgtgattagggaccaaaaaacactgtgacatttggtttcagtctctcagaaccccattccactgtcaaacacccaggtgttagtcaacctgcttgggaaataattatgcctggaagtttcaaccaagagagaatagcaagtgccaaactattcacacatatataataatatgcaggagggtacagtctaaactgatgaacttacactgtctccaaagataagtaaaatcagacttcatcattctaaaaaaaggaaacaaaattaaggatgatttataaaagaaaatccaaagattttggtgaagtctctgttcaccaaaccttaaaattcaggagccattatttagtaattttcaggtgatataagaaatataggaaaaattggtcttctggtcaggactaattccagaacataggcacaaataatgttttcaatttcagattagcggttacagatatcagcagactgaagttattgatacctttctctacttgggaactgtgttttaaaatttaatattgtgttatatatatcacctttatttatttggtaatattgaaagtaaacttacacagaatgtaatacaggttggacatccctaatctgaaaatttgaaattttatatgctccaaaatctgaaacttcttgagtgccaacatacggccataagtggaaaattctacacctggcatgtttgctttctggtggtcaaaggtacacaaactttgtttcatgcatataattatttaaaatactgtattaaattaccttcaacctgtgtgtataaggtgtatgtgaaacataaatgaattttatgtttagacttgggtccctagtatgtctcattatgtatatgcaaatattccaaaatctgaaatctgaaatctccagtgttttggataagggatactcaaaacattgctctgcagaagtttcttgttaccagtcttatttttgtataaatcaaatttgcttgcctgagttttcctttaagtttatgtgaaagtgaattttataagtaacttattcgaaagaaatactgtaataaaaattaaaaactgtggatgggggaaactgaagtgttaggatataaaagataaatatgaggcaatatatagttaaagtctgaggattcagcttcaaggctagagatttccatcattcaaaaagattattcagctattggatatactaatattttatcactgtataccttattcacatttaattattatgtgaatcagttgatcctggttaaggactttttaggtgttctttctttccctctctacctatacagtttagggagttttaagttaaaatattccaataaaaagtttgtaagtttgtaataaaagtggtggcttagagctatctttttcttcaaatagttcttaaagtgaaactgtgtctaatacgtattagagagctctggtttagatgcctaaaacgggtcctcctggccagcagacccagtacctgtccctggcctgttaggaaccgggctgcacagctggaggtgagcagtggggccagcattaccacatgtgctctgcctgttgtcggagcagcagcggcactagattctcatagaaggaccaaccctattgtgaactgcacatgcaagggatctaggttgcctgctcctaatgagaatctaatgcctgatgatctgaggtggaacagtttcatccccaaaccatcgcccccacacttccccaatccatggaaaaattgtcttccacgaaactgctccctggtgccaaaaaagttggggaccactggcctaagggactaaattccccttcggactgctactttgtatatgtatatggtgggggacagattgtaacacatttttgtgtctttttctatattgatacaataggaattgtaatgtgttgcttgctaagtaacataatgagataattcaaataatcacaagttgcataaaactaggaaaaaatgtgcacatactaattattattttaaatttatatttaacagaagcataatattttatatttggcctgacaatgtactcatttctcaagcatcactttggtagttagcttgataatttaaattgtactcatttataagaggttgtgttttagcaacttttgtacattattgcgattgttacaaaggggtctttatgatggctagcattgccatttgaaataaattagggatttttaaaaatctataagaattcttacagactatgcttgtcaggtacacttcggattttaagtatttgtatagtaaagccaaggaaatcagtgttgtgatgtgaagaaaaaaactgtatagggatagaatcattttgctctaatcataactctcctaactcaccaggagttagcaaggcttagtggacgaatatgtcttataagtacaacaaaaatataaggcactattatgaagaggattttaggcatatgtttttgaacaggaggagacaatgttgtgtttattgtatcctcagcacatactgtagtgtccaaatgtaggtgatcagtaagcactgatctttgtgacatagaatcatatttagaaataaaaatactttcttatatttctatcaggagccatacgctttatctgaaaagcaggtaaattgctgtgtgccagtcttatttgatcatttattgagcacagatttggccctttgtattcgtgggttttgcctccatgggttccacttaccatggattgaaaatattaggaaaaaaaaggatagttatttctgtactatgcatatacagattttttttggtcattattccctaaacaaaacagtagaacaactatttacataacatttacattgtattaagtattaaaagtaatctagagatgatttaaaatatacaggagtaggttatatgcatctgctaccccattttatataagggacttgaacctctatggattctggtatccacaggggtcctagacccaatctccaattttccatagataccaagggataactgtatataagaaacacttttgtataagttattaactagttgcctatttctattgatatttggagacattttggcaaagattgttaggttacagagaggctgtgtgctgtggtgaaaaaaaattcgatcacgaatcagaaaacacggtttttccattcatctattaactagctgtttggcctaagatcttggcctttcagagcttgtttcttcacctgcaaaatttctaatacaactcccaaatgctgagattcctagtatgttaaagcctgtttttttatttcagggaagagtaagtaaatcactccctagagagaagacagtctagctcagtttgttgtattttcagaagctaagatcattttgtctgcctgtttataaatttgcacatattttaaaggtccttgtgtgaaattctgtggcagagaaaagctgccattggaaaagaatggagtggaaggattttccccccttttttgaaaccgttagtgactttggagaataaattgctacaggtgtggtcaaaattttggttactggcactgggagcaattaaatgactgccagacattgagagtaggtcatctactgcacaccagttgccagggttataaaggccagcagacttgtcacagcacccttaatagccactggccatcatccttacttgttaaagacttctaggaaagtatattccatatttcccttggtaactcatccctctaggtggttttcagaaaatgtgttctaatcttagaaacataaatctgtaggcatggtaagcctcttttttctaggtctatcctcagttgtgcagataagtatgtggaaagataacagaaatcattgtatttgatcagaaagcagtgatgtttatgctattgatttattgtttttttctgttaatattcaaattaccttcaacttttgaatattttttaaccagcacactgctcaggtaatgagatattatttagagttattttgaggatgtactcatttttcaacaagctaagcatatttctgcagcttgtttttctttgagagttcttgcttttccaagttctttttcctgatggacttatggcaggtctagacctgaaagtactattgtatttcaaaacaactgactgcctttagccccacagtgactgaactgtatttttttcttaggaggttaatgtctttggcaagctaaacatccttggaattttttgctaagtcagccactattttgtgaatatagatttatggagtatatcatcaaattcaaagcagcattaatgggaaaatttgcggaaaaaatattgaaattagacactacttacaaactccaatctgaaatgaaaccatacatgacagcagggaaaggaaagaattacattttggaaccctaacttttatgcatataaatatcaatgcatagtaatgtcattttaaagttatatgccttctgaagcttttcttaaaatctaatggctactattatttgcagttgagaccatgataggcagcatcttatggtatttatgaaagtgatatttgtgttcttttttctaactatataccaattgtaaaagttgaacatgactttttcaaagcagtttctatacttgataataacagtggtagtaaaactcagaacagtttaaaatattagtaattttacagtgtagagacttttcttcactagaaatgaccagctttcagatttcttagtgatattatgagactctagatttcttcaaaatcattttgagtaaatctaccagtgtataactgctaaagcacagttaaatgtatcatgctgtggtcttttttgtttgtttgtttggtataaatatcagacatatatgtttgtttactctaaaactaattttttttaccaatcaaaattgaggcacgtaatctgacaatgggacaggatatttgcagtcaggatagtcttagaaaagccacaaaatatggtcgatgtacttagacttctttaattttccttgccttttcctaaatatatctcataatgattaagagcatgttctctagagtcagactatcgagattactagttttgtaactttgggaaagttatttaacctgctaaatattctccgggtaatacgtatttcttcatttgtaaatgaggggtaataactacctacatcacaatgttgtaatgaggattaaataaataatacttgaaagcatttagtaaagtgtgttgtacatagtaagcactcagtaagtatctgtgaactccttgagagtaagaaccatgttacattcatccatttatttgggtgctttaacattatgaagtgaattgaattctgtcccttccaagaatttaccagtggatccgatttctggatagtgggcaataaggttgccttcaaacaagtttagagtagttttcagttctttacaatactgcttgctttttattcgttaaaacttttaaaatttcattctagctctaggaatctgaatgctatttcatacctactcctaagggaagttagcatatggatatttagtttaataggaatgaggaattaatggacagaaggtattaagtaagtgaatgtacaattttcacatctagtctgtgcttcctgtggattgattttccctgtccattcctggaatcttcgaattcccagcacttatcttaatagcactctaccattgaagcagttttgttttgtgctgttagatacttcctttccctgtcttgtctttttttcatgcactaaagacaagagttgtgtcgtattgtcacatagtccttatattacagggcttttaaagaaatctcacgtcctgttttcaagctgcttgtatcttgtttgtccattctacttgctatgaagtctttattgaatataatattaaactcttttgcatatcattgtttgttacctaggacactcaatggacatgtgtaaactttacaagtcatttatgtaaaaataaggaaagcaaccatgtgttttaattatttccatcagttcaatctctaagtctttgcctttaattaccaaacagctatgtggtgccactgatacgagtttctgtggtatcggctacctggttttagtactgttgttactactttatgtaaatgagactattcactcatatttaagtttgtggaaaatgtatatatgttcttgcgttttagcagttttggctagactgctcttgatggtcatgcagggtaacttacttttctaaagttgatgatttcatggtctgtatttctcatataccaggataatagtaaaatctacacactttaaaaaactacatatgttgttgttaacatatatggaggtttcttcagaaattatctgcgatttttgttgtaattgttttcattgttgtgtgcttttaaagaatgacttttattaagcatgcttccaagcttttttgtacatgaaaaactgcatataattttgtctcttttagggggtgatatgtacaatgaaaaaataacatgacattggattgccctggggctgaccaagagtttgtttccatagtaagggattatgactaatttaggcattgaagttaagaatttattacttatttttaaatgacatcacgaagagttacagctgaaacccatggaagaagttttcttgatttttctatatgtacacacaaatgtgtattttgaattcccataaacccaaactttaatataaacctgaggagggggagcattatggactttgtatatgctgcctcccttaatcctcacaataacacttctgatacatgtgtaatcacttcatttccatgattaggaaaatgaagtcccacatcaaattaatacagtagaatctcacctcatttattttacatgaaatcaactgtacctactcagtcaaacataaaaacgtgacaaaaataatacagtcatgtgtagctttacagtggcaatatgttgtgagaaatgcatcattagatgatttcatcattgtgcagacaccacagagtgtaccgacacaaacttagatggtatagcttactaaacacctatgctatctggtacagcctgttactcctgggctacaaacctgtacagtatattactatactgaataccataggaaaatgtaacacagtggtatttgtatatataaatatatctaaacatagaaaaggtacagtaaaatacggtgtaaaaaattttaaaaatggtatgcatgtataaagccttaccgtaagtgaagcttgcaagactagaagttactctgggtgagttggtgagtgagtggtgagtgaatgtgaaggcccacgacattactacacactaccgtagacttgatgaacactgcacccttaggctacactagattttttaaaaagtaaagtaattgcgctacaatgttatgacagctatgatgtcactaaatgatgagagtttgtcagctccattaatcttacgggatcactgttgtatatgtagtccacagttgaccagaacattgttatgtagcacataactaatgtaaatattgcaaatcacatctgactactgagcttttgcttcaaagtagacacttaaagataagaatttgccatcctttcagttggtctcagtgcctaggtctaggtatgttacattttgttgtgttgagtgtgattctgttattcagacatgtaatgttcatacaacatggcctctaatacccagcctgtagtgcccaaccaaagaaacagcagtttgatccggtgctggaagagaaactgctgtttgggcttgctgagcaattgtattggtctccaatgtggcctgcagatatggcaacaagaaactgtcatgtattctctttgatatacacacgtgtgcaccccacatgtaagccttcacccttcttaattctatgtatattttatatattgtgtatatagttctattgttttagattgctatatgtgcattatatctttgtacaacacacaattttgtacagtgtggtatacaaaaaccacatacattatactttatggatgacttgagatttttcaagaaaaatgtcgattctgtacaatcatagactctgtttgtttgcacatactcatttctagaacatggcaccccagaaatacctctttcagttactagctacttagtaccaacttttcaggctcacaaaacagtagtttgttcaatgttacatagtaggcaaattactattatggttctgatctagaattataagcaaacgtgctgtttcattgcatcagaccttcccttcttcctcggtgtaagagagatcaagtcttcttggctcagtagcaatgtcttctgtccagctagcaagaatggcacaggaggctcagggtacacttctcattacaaggccttaagggaagtgctgcttctccacacagagcagataggacaaaaattgggtgtttgcacaaagggagcaatgattccataaggaggtgaaagtaacacatagatttgtcaccttatttacattttctatcttcttgtctctaggcaaagggggtaacagaaacctgcttagtgtttttataaagtaaattctgcctagtatcattgaatttcctttagcacatccatagacatacaacagaacagataggagaaagaaacataaataacaggaagtgctgggcgtggtggctcacgcctgtaattccagcactttgggaggccgaggcgggcagatcacttgaggtcaggagtttaagaccaccctggccaacatggcaaaaccccgtctctaccaaaaatacaaaaaattagctgggcgtggtggcgcgtgcctgtaatcccagctatcgggaggcttaggcagcagaatcgcttaaacccaggagttggaggttgcggtgagctgagatcacgccactgcactccagcctgggtgacagaatgagactctgtctcaaaaagtgataataataaagagataacagaaagtccagtggtcacaggaccagatgttttgagatgcttgtaattccaaatgttccatttccatactgttctaatatgactcaagtaattcatctggggaatgaaacagatttttcaaaagcgccttcagaatttgggctgagaaaatatcactatatgtatatttatatttttttaaaaagctgagtgattaaacattatttcctgtttattcaatataccctataattgacaatgtggtagaccctgggtatataatggtaaacaacacaaacatactctgtgattgtacggtcaacgttttccttgaaaaatctctttaattactcataaagtacaatgtatatagtttttatatatcaccttgtacagaaatatacattttatctctgtataaaacatacagaaatctgaaacatgatgaagttatatatgcaatgtatgcaatacatgtatttaaaagtgtgaatttatgtgtatatatatatgtatcaaagagaatacacatgcacagatgatagtttgttgtggccatattaagccatctttgcattttaaggataatttctacttgtagaaattatctacaagtagataaacacattatctaaacacatgttgtagagtcacactctgccacacccatctaatttttcatatttttggtagagacagggtttcgccatgttgcccaggctggtcttgaactcctgggctcgagcagtctgcctacctcagtctcccaaagtgttgggattacatgcctaagttagacttaatatcatacccggcctggtatgataggatttcattcctaggaaaaccaagagtcaactgagaaattgttagaatgaataagaattctagagtaaatagatgtaaggcaaatatttaaaaatcaatagttcttccttatgatagcagtaattgtttagaaaacgggaaaaaaacccatttagaatagtaacagaaaatataaaattataaatgagccaatgtgaagaaacctaaaaatctacttggatgttggaggttacagggaaaggaatatcatgtttctgaatgggaagtccagcaattacagataccagttttcctcattaaattacaggtttagcctaattctcttctttaaaaaaaaaaaagtgagaaattagcaaggtaatttttaattttgtctgagaaactataaacagataaaagtaaggtatcttttgagtggaaaatgaaggacagagcgctaaccctgtagcttattagctatcaaaatatacaatacaattagagtaaatgaaagagttgaatactggcatacacattgatagtaaaattggtaaaagggaatagagatcccagaaacactctaaatatgataaagaagtaatcagaaattaggagaaaaaggaaaactaagatttgtttttgagagtattaaatacttaggaaaatagacttttgaatctttatttcatcttacataccaaaataaagtctaggtaagttgaagagttaattggaaaagagtaaacttatgcctagtgtcattttaaaaagacatttatgtgaatggggaaatactttccaaagcttcaagaattgtaagtactcaaaaggaggaacaatcattcgttcgactctcaaataagattggggcatggcaatgtgccccctctagtccaagctactcaggagactgaggcaggaagactgcttgacccccggagtttaaatgcagcctgctagcaagactccctatctttaaaaaaacaaaaaaagaaaaaaatctctttctctcacctcctctccccccaacccccatacaagtgaacaaattaaaagatgtgcagaccaggattagagttctgcaaaaattcaatagatgtttgtctttaaatttttactacaaatactttcaaaagcaccatgacccatatggatgaatgagcaaagaatttgagaagttgggctcatttactaaagaggaaatgcaggtacaagcataagggacatgtttttccctgacaggtaattgaagaagtgaaattttaatatgtagtgatttttttttttttactttttgagttagcagaagtatttttctaatgatgtttttgctggtgagggtgccatgcaatgtgcttgtccatttatttccattgacagtctgaattggtttaaccctttcgaaaagcacgtgaacaatttgcatgcctttaaaatcaacatacagtttgacccaataattctctttgcagcattagatataataatttacaacagcttatgtatttaacaactaaactgtttaacatctcccgtggaaataggttgtcatagaaaatgtttatgaagagtttacaataatttgcggaaatgtttttgtagtgaaaaaagatacaaaattatgtatactatatgagtacaagtatacaaaactatagaaaacaaagaaataaaccaaaaatatccagacaaaactgtaagagaatacataaaagatgctaatatggtgtcttaggtacgttttcttttggtttcatcattcatctgtgcaccgctatatttccgatacttacagagcacaatcccaggcccataatagacctttcagtcagtgcttaagtgaataaatttctgtaataaacatgggattcatttacattgaaagaaatatgaatacactttccatccaagttaagaaaaggaaaaatgtgggaagcactctagaaaggaaactgattaataccttacaattagattttgttttcagaatgttttcacatctctattttatgcgatccttttctgttctaatttaataatatcattacttacctaagttagaaatttgtgctaaccggctgggcgtggtgcctcatgcctgtaatcccagcactttgggaggccaaggcgggtggataacctgaggtcaggagttcaagaccaagcctgaccaacatggcaaaaccccgtctctactaaaaaatataaaattagccaggcatggtggcgcatacctgtaattccagctactcagaaggctgaggcagaagaatcacttgaacccaggaggcggaggtagcagtgagccaagattgcgccattgtactccagcctgggcaacaagagtgaaactctgtcttaaaaaaaaaaagaaaaagaaaaaagaaatttctgctaaccttgattctatcccttttgacctgccaaatccaactgattaccaaatcttgggtattctttcttagaaatgtctctggattttagccccatcttttctcataatcactgccttagtttattcctttctcatttcactttactgtccctacctgtccttctacctttctactctcctcttttactttccacactgctgtaaaaattgtcaggtaaatagcagaattttgtgtgtgtgctattataaataggctgtatggaagagactggggggagattaacactgctttcacactgctattacaataatcaccctaaaatacaaaattgatctgcttaaagcttagtaattccttatgacctatattgatgcttctcaaacttgggttgtataagatcacccaggagacttgataatgtgcaaattatccagaccctagaaattctggttgagtggatccatagtactgggaacctgaattttatttttattttaattttttgaaacaagagtctcactgtgacacccaggctggagtgcagtggctgatcataactcactgcagccttgaactcctgggcttaagtagtcctcccacctccacctcccaaagccttaagattgcaggcatgagccactgtgttgagcctggaacctgaattttaataaggcttccctctcatgatccttacgcatatgatcccaggacaagatgttgaagaacagaccttgggataacatgcagtctcattaatttactgtaattctcacaatttggcatcagccttcttttctgtgctcattttctacatttttttctcatggacaccttacagtctagtcactctgaactgctacatgctcacctaggttattgtggacccctgttcttgtgctcatgctgttctgtggttcacagccttttttgctatggcttctcctactcagtaacatgttgatcttggtacactatcttcagcttatatgtaatctgagtaaagtattcccagactcactctcctagacaaagtctatctctccctcttctgtatttccatagcctctattacaacacacatatactaaattacacttcttttcataccttcctcactagaccatgaacctcttgaagtcagggatgccatattttagatcttttatttaatcttagagtaaaagcctggcacaaaataattgcatgctaaatatttgtcaaatcattaacaatccaataaagaaggcaaggagtaattagtcccattttcagtgaggaaaatgatatcttgagaaattaagtatttgcttttaattacccagcttgtaagcgtaggctcaagacttacacataagaaatctggcttcttttctggttttggtctttacagaaaagatagagaataactgtaaaattaccccttccatttttacccacacggctcacatgttcatttaaattaccccctccattttctacccataaagctcacatattagtttatgttttatcattttatgggtaaaggtgttttaataaacttttacatcatattcacactttataaaaataaaataatgcctattcctctagggtatgaacagattgctggatattaagccatttgtctctcactttcacatctaaccttctttactctgctttgtaatgcttatttatttctcttgcagaaagcatgttatagtgccttatgggatgtctacaaatatttgtttaatcaaattgagctaatatttatgtaggaaacattttattctgcaacaattaaggcaaacagatatatgtgtgtaagtattcacatgggtttccactgtagttttgaaagctcagtttccaaagtttagatgtgtttagccttactgtagacttcactcttaaggtgttgatggaaatcaacaaccagctgcaaaattcagatccagtatgccataggatacatgtagccacggtttaaaccaaatgttagcaatgaaatatttagtagcaactgttcttttaaaagaaacattttagataattagagtcgtcttgtaatatcttatacccagctgtcagctttttactttttctgccaatatttctggaactgaaaccaagaaaatggaaattagggcaccacactttgaaatgtattaaacacttgaaaaactgttatatcttataggatttccaaaaagagttcttcagtgcctcagaagatttttataaaaagcaattatctacaataaccctccccagtattaagctgaaatatacacataccttagtaaataataggagcatttgggaagtggaagaattgcctcttaatagctactggacttgaagattattaaatttgagcattctaattccatttttgaaagtttctgatattgtttcttaattttgttaccatgtttaatcagtatctcactagcctttcattcatttctttagcttctgttactatctttctacttttaagatactcatatcttcattttctgccacatcaactttcagtaatattcctgattcctcactatgttagacagtgataatagtgtctcattcttcctttcaagtctttcccacccttctacaaaatgttatatataattactttttaaattgtcaagtattgataacatttgcattctgttctataaccatatttaaatcctctttctttttaactatagatagattgtaatagttgaacctagtactatttacattaatattatataaaaatgtttcactttacaaccaagtagagtgctacagttggatttctttctctgaatctgtgtccagcatcatgaactacgcaatgaatattctttgcattcagatcatatggtttatcttttcatatattccacatactgctcaaaattgtgctcttttgcttcatttttagcatgatgttatacattttttgttttcccctaaagtttccaattacttttcttttttctattgtgaaaaccataaaaacggtattcagcgtgccattagtgctgctggactcctcagttcttcatctgttgcttcaaacctgttgtattgtttttcctgaatactttttactctggaattcagatttcatcttcaagtttgggcatattactttctgtaattaaagtttatctcaggactgctttttcttaatgatttgtttacgttatttgaccactggttcagttccaccatttctagcatgtggtctcttcctctttcttgacctccacctttggttggctagagtaattaatcaattaagtgccttagaattggttcaggggaagtaaaactttggaaaactaattgtctgaaaagacttttattttttctttacacatgatagtttgctgagtatagaattcctggtcaaattgttttccagcattttaattactgcattgttttttacttgttattgatgagaatagcattgccagtcagataatttttcttttgcagattacttgtttttccatctctgaaagcttttaagcttttatctctatcttttattgtctagtatagttctttacatttattctgcccactggtcccttttaatctaaacatgtaagtctctccagctcccagatgtttttcttttatatctttaatttcctttcttccatttttctcttacctcagaacttgtgcaataaggaagtattcagttgttgaacctctttgatttattgtatatctttttcacccttgtttcattatgtctgtctttttgctctatgttctagcaattttttaaattttgcaaatataggtgaaatgttatatccttatgattttatttttatattgtgtttgttattctattaagattcatttcaatataaggtataaattggaaacccactcttttttcccaaatgtctagccgtttccaggtggtctcttccctattgatttgaaatgatacctttatcatgtaacacattctgtgtctatttttttaatttctgggttttttttccttttgtctggctctttatgagccgtatcttaccattgtgattattgtagtttcataatttttttttctagagaagagaaggtttggttaaacttttgagatgtttatttattttcccacgtcaacaatcatggactatcatgttatgtttatgagtggctgagtcatatctcctgaggaaaacatagcaccattgagtgctacagcatcctatagagaacttcagtttttggtcatactgttaagacctcttggtttcacagccccacaagccctctttgaagtgacctatccagtagggctagacctttctctccccctgccttctcccctcccctaatttttctggtctcctgcaatttttttttaaacattagaatcagcatgcttaattctacacacaaacacaaaaatcctattggcattttattgggattacatcatatgtgtagattatctaaggaagaattgacattttgggaattggttgtcttcctatccgagaatgtggaatgtttttccatgtcttcaatcttttgtgttacttgggagcattttaaagttttcttcgtttattttaaagcagttaaaattcattttttttcagttcacgtgactaatcaattctaccaggaccgtctattaaataatctgtcctaattaaggttctggggtttttccctatacaaaattgcagcaactattgtacttaagtttgtttctgaactcagttctgttctctggatctgcatgggccaatactattctatttttataactaaggatttaatacctacttaggctagccccttctcattactcttctttcaaacattcagtatattaaaagctgttataatacttcttactctgttttagaggtggacatagacattacacacatacaaaaaaactgcaagccaatctcacttaaagtattagttatcttaaataaaatattagcagattcagtagtttgttaaaacacacacacataccatagccaaatggcatttattctggaaatacaaaggtcattttaatcttaggaaatctgtaacagacattaattctctctctttccctgcctcccttcctccctccctcttcctttctccagcacagacatgcacatgcgcgtgcacacgcgcgcgcgcacacacacacacacacacacacgaccttagtaaaatagggatagaaagctattctttgacgtactaagtaatagatataccaaactagaagcttcagtcaggcttaataatgaaagactcgcattgtcattcatattacgaataattcaagaaacaatcaatagctatttttttgccttgcttttgtaagggaaaaaagtaatataaaggagaagacatattcgcaaattttaatttcctgtatcagtttctcctgaccactcactcacattctccccaaagtgaagccaatgttggtaacggagtgaattctagtgggggacattggaggggatatagaaatacgtttgttcatatatgctttttaacacagatttttaattgtattagtataaaagtgtgataaacatgctttcattttaaatatcttctgaacctataaattatattccccttttaaatttttgtttaatttttatcatcaaaataaatgtatcttccagtaaggacagtgtgaggaggttggatgatttccttcttcagaaaacaggcatcaaaatactggagtaactgtgttaatgtcagccaaaatagattgtaagaaaaggagtattattagagataaagaggaacatttcaaaattatgaagtgacaaatatggcaggaaaatataaaaattctaaatgtatatgcacttaagagaggcacaaaatgcatgaaacaaagattgatagaactgagagatgaaataggcaattcaatatttagaagagttggagattttaatactcttctctcaacagttgataggataagtaaacagaaaacaggatacagaagacaggaataaagcaatcagccaactcaatctaattgacatctgcagaatacccaacaacagtagaaaacacattattctaaagtgcattggtaacattagccagggtgaatagtatactggcccacaaaaatgaaatgttttaataaatatggaagaattgaaattatatggtatgttttgcaactacaaaggaattaaaaatcatggagagaaataaggaagaaataagtaaaatccccaaatatgtgcaaatgaatactatttctgagtaacccatgggtgaaaaaagaattcactgggcaaaatagaaaatatttttaactgagtgaaaatgaacacaaaatgtaagaaaatatttggtatgtagctctcaggcagtgctaagaaagaaatgtttggctttaaatgcttatgccagaaaggaagaaaggttaaaaagtcaattatctaagctactaccttaagaagctagaaaagaaagagtaaagtgtaccaaagtaagtagaagaaagggagtaataaagatcagaacagaaatcaaggaaatcaaaagctggttctttaaagagttcagtaaatctttagctaaaatgaacaagaagggatgtaaattagaaatactgagaatgaaagaatgggtattactacagatactataaatactaaaaagataaaggaatattatgaacaagtttacaacaaccaatttggcaacttagatgaaatgaacaaattctttgaaagatactaattaccaaaactgactccaaaagaagtagaaaatctaaatagacctatatagagtaaataaattgaattagtagtttaaaacttccaataaagaaaatctgggctttaatggcatagctagtaattctgtcagacaccaaaggaagaaaaaaatacaagtcccaaccacattttcagaaaagcaggagggaggagagcatcacaattcattttatgaggacagtattactagataccaaagccaacacaaaggcatcccaagaaaagaaaactagcagccagtattcctcataaatgtagacacaaaaatccttagcaaaatattagcatgtttaattagcaatattatatttaaaaggataatactataatcatggtcaagtaggatttatcccagcaatgcaaggctcacttaacttagaaatatcaattctgtgtaatataccatactaataagatatatttgaaaaaaaagtgatcatctcaatagatgcagaaaagtatttgacaaaattcaatacaaattataataaaaactcttagcaagctaggaattgaaggcaacttcctcaacctgataaagaatgtctgtgaaaaaaaaaatcatacttcatggtgaatgattttatagttttccacttaagatgaagaacaaggcaaggatgtttgctcttactgcttttattcagcattatcctggaggtcctagctaatgcagtaaggcaagaaaaagaaatttaaaggatatttggaatggaaaggaagaagtaaaaccaaatagatggcatttatacagaaagttctaaagaatctaccaaaaagaaaaaaaaaaaatactagaactaacaagtgacttaggcaaggctgtagagtaaaagataacaaacaggaattaattatatttctaaacactagcaatgaaccatccaaaaatgaaattaaaaaaccatttacagtagtattgcatagctaaatacttagtaataagaatagatacacaaacctgtacactgaacactacaaaatattgttgatggaagttaaagaagaactaagttaattgagaggtacactatattcatgaattggtacactatgttcagtgcagtcccagtcaaaatcttgtcagattttctcatagaaattgacaggctggttttaaaatttatatggaagtgcaaaaaacataaccacacataaaaactataaaatctgaacacttacaagtacctgatttgtacttgctataaagctacaataatcaaaatagtacagtattagcataaggatagacatatagatggatgaaagagatttgaaaatctagaaagagtaattcttaatatagtaatatttttcaattaatttcttattttggacaattggtatatcctatagatctttttcccgtgatttaatgaaatgtaacatttataataattttctttttattgaacatttttgctttgtatattatatgttaccttatttgttaataaaaacacataaataaacttggttaggtattatttatgtggtaactgattttcaacaaaggtgccaaggtaattcaatgggaaaagaatagtcttttcaaaaaaaagaaaggccgggcgcggtagctcacgcctgtaatcccagcactttgggaggctgaggcgggttgatcacgaggtcaggagatcgagaccatcctggctaacacacagtgaaaccccgtctctgctaaaaatacaaaaaaaaaaaaaaaaaaaaaaaacttagccaggtgtggtggcgggcgcctgtagtcccagctactcgggaggctgaggcaggggaatggcaggaatccgggaggcggagcttgcagtgagcctatatcgtgccactgcactccagcctgggtgacagagggagactccatctcaaaaaaaaaagtgctagaataacaaaaaatttattaaaaatggattgatggttcatagacctaaacttcagttcttaccccagtaattacctacgtggtgtattgacaacaaaggaagaaaacttgtagaggtgggttttggcagtgtttttttgtttttgtttagaggtggagtttcactcttgttgcccaggctagagtgcaatggcacaatctcggcacactgcaacctccgcctccctggttcaagtgattcttctgcctcagcctcccaagtagctgggattacaggcccctgccaccacacctggctaattttttgtatttttagtagagacagggtttcgctatattggccaggctggtctcaaactcctgacctcaggtgatccacccgcctcggcctcccgaagtgctgggattacaggcgtgacccattgcgcccggcccggaagtgttttaaaacttggttgtagtggtggtttttacaactatacattcaccaagactcatcaaactgaattttaatttatacctcaggtaaagctaaaataaagtaatacatgcatattattttcaaattacaactgaaaggttaataaaaacatcaggccgagcatggtggctcatgcctgtaatcccagcactttgggaggctgaggtgggcgggtcatgaggtcaggagttcgagaccagcctggccaacacagcgaaaccctgtctctactaaaaatacaaaaaaattagctgggcatggtggcaagcacctgtaatcccagctctcgggaggctaaggcaggagaatagcttgaacctgggaggcggaggttgcagtgagccgagattgtgccactgcactctctagcctgagcagcagagctagactccatctcagaaacaaacaaaacatcagttcattactgtatctcacttcccccccattccctctcacaactttgtcttgtctcatgtcatgcttcaggaaaccactttcaactatttaagctactacttctggtatttgccaccttataataatggggtatattgctctgtcttggtccaccaatatttgacattattcattagttttctcttataattttaatgctcatatcttcctttgccttatctcatcatcctatatttaatcattactggttaaatccacatttagttatttcattattcaatgaccatatagtaataaagcaaatgctacatggggttatattttttctgttacaactttcttgttgttttttaattaattgcttcatctcagaaatctgcttagttaccattgtgtctgaatttgtctttccatgggcttcaatccatctttgcaataactctcacatgtgataattagtttggttattttcctataatatccttcatagagccttcagtatttctgatttaatcttgattgcttgctaggtctgtttcacagctgttatctcaagactcttttttgtcttcatcctggaaattccctacacctgcttcctgtgttgaagcattgattcctcctatgccccataccttcatggttgactacctcattctataggagcttatcctctcgtggcatcttgaagagagataactgggaggtaatttttttgagattttacatacctgaatatgtgttattctactcctacaattatgtgatattttaagtgagtgtagaatgctagattgaaaatcatttttacccagatattgctctggtatcttccagctttcaattttgctagagttctaagtgccactctgtttcctgattttttatttattcttttttctctctgtctggaagtgattaatctcttctcttcattttatggtatggtaccttggtgtgcatcgtttatctgttgtgctgtgtactccgtaagtccctgctgcttgcagacttacattcttctcggaagtatccttgccttttttggtagttccctctcctttgttttcattattctctctggaactcctactagccagatactacacttcctagattgtttttctaaatttttaccattccctcctcttactcatccctttctttttattctgccttctggaaaattgccttaactttaacttcaaacatttgttgattttttttttttttcagttcttattctctggtcttattggttcctctgcataaggatgcagtcttttatctttacagtttctttgacattttcctctgtcccatcattctctgtttctcaccacttctgtttcattttggtctcaatctttaatattggagacattcctcaaatgcatgaagatcctcagtggtcatttatatttaaaagatgtgaaaagctgaccgaaagctctgggtgtgaagtcagagctcttgtctattggtaaactatgctgtaggaaatcttcatatcacaatttttctttaggttagttttgttttctctagttttgaatcctttccagaggaaatctatagtcttctctgtgggggctgcttatgttttagagaaatactgaagaaagggactttgggtctctttagagttgcacataatcttctggttttagtcatatctacttctgtatctattgaagtccaaagcatcttgagtttagtttttccagaaattatgccttctgatttctgcatgattgggaagtcactgagtacaccaactggagttggagacctggaattccagtagttccaggaacctcctagtcctgaacttaatggggtttcatgagaattgactggcttctttaggcacttaaatttaacattcctcacctctggtgaggtttttgtttgttgtttgttttttctccctgtctttgtatattatggtttagggttacagttttccagcttcatcaaaaaaagagtttttctcattctccttgtttagaaatgattggtgacacaaaggtctgtactctgatattttaagatccgaagtgtattctaccccttttttataaactattttgcagctgttccttcacgactttgctacatcttggagatcttttcatgtcagtacctacaagtctatatcattgatattatcagctatatagtactccataaaatgctttattataatttatgtaatcatcttcctattgatgaagattttggtggtttctagtttttgctttcatgaatattgttgcaatgaatattgtttggcttacttttgggtgaatttttacaaagacatccatataataagttcccagtagtggggttgcctaatctaagaataagtgtatttaaaatttgaatcatattgtcagactgtctcccagaaagtttacattcctaccaacaatctgagagagctgtctttgcagttactaggtttcatcaaacttgtttttttcagtatggtaggtttaaaaatggggatacatttttgtttttatttgcatttttaaatattttcttaggttagttggctacttaaatttctttttctgaaaactttgtatttatagccttttaaaattcttattgacttgcctgaactatttgtaaattacagaaattagccctttgtcgtatgtgttgcaggtgcttttccagtttgccagtggtcattcattttggtttatggtacttttgatagaccagaatctttgacttttatttagtgagatttatccatctttttctgtgtggcatcttggtattatagcattaatattctcttttcttttttttttttttttttgagatggagtctcactctgtcgcccaggctggcgtgcagtggcctggtctccactcactgcaacctccgcctcctcgtttcaagtgattctcctgcctcagcctcctgagtagctgggattacaggcatctgccaccacacccagctaatttttgaatttttagtagagatggggtttcatcatgttggtcaggctggtctcaaactcctgatctcaggtgatccgcccgccttggcctcccaaagtgctggaattacaggcatgagccaccacgcccaggctatagtttttctttatctgaacaataactaattataaagtatagtggaagagaaaaacccattcagaaaattaacaaaaagatcacttatctagaaataaacttaaaccatagcagagcttacatgaagaaatcttttaaaactgagcaaggatttgttggacaggacacagcactaatctcaaaagaaaatttgatgaataggtcttattcatagtttttgctcatcaaaagacagcattaagaaaatgaatagacaagccacagaatgagttaaaaaaacagaaattcccagcatttacaagggattaaagatgtgtatcagaatatataaagaacctctgaaggccaggtgtggtggtttacacctttaatcccagtgctctgggaggccaaggtgggaggatcatttaaagctaggagttcaagaccagcctggtaacatagtgaaacaagattctgtctttacaaaaaaaaaaaaaaaaaagtgtttgggtttttttttgtgtttttttttttttgaagttagctaggcatggaggcatgtgcctatagtcccagctattcaggagcttgaggctgtagtaagctatgatcatgccactgcactccagcctgggtgaccaagtgcgaccttgtctctaaagatcaaaaaaaaaaaaaaaaaaactcagaaattaacatttaaaaaattatgaactacttaatataaaaatgggcaaaagactcgagcagacatttcacagaagatatacaaatggccaataagcacatgaaaagatttccaccatcattattagtcatcagtaaatgcaaattaaaaccatgataagataccactacaaattcaaaccatcatgagatacccactagaatagctgaaataaaagtgactgatactactgaatagttatgatatagaccaactggaattctaacttattgctggtgggaatataagaactatttcttataaagttaaacataacatttacctaatgtgtcaggaggttcccagaatcatccctaggttccatggttcgctaggacaactcacaggactcagcatgatgtcgtactcatggctctatcacttactacaatgaaaagacacaaagcacagtcagcaaagggaaaaggtgaatggggcaaaatccaggggagaccaggtacaagcttccaacaatcctcttccgtagagtctcacaagacacacttaattcccccagcaaagagctgtgacaatacttgtgaaatctaaccagccagggaagttcgctacagattcagtgcccagggcttttaccggggtctgatcataccctctgctcagcgcacacccaaattccagactcccaaaggaaagcaggtattaagcataaaccatattgttatcataaatgctttagacacatggagccagtcttatcagatgggaatcagggtaggaatcctcctaaaatccaagatccaagttcccagacaccaagccagccttgcaaacaggcttttttaaggataagcataagaatattaactcttttctgcaagtttacaacctagcattttgctaagtataacaaaaattgatgtaggaattttgatagccgatgctggaaacagcccacatattcaacaactggaaattgtataaacaagctgtagtatattttggaatactacttaaaaagaactaaccactaatacattaaaacattgatgaatcccaacctgttgcactcttctcattctgtacctgacagtatgttaaccaaaagaagccaaatacaagagtatacactgtgattcaatttatgtaaagttaagactcagcaaaactaatctataattacagaaaccaaaatgtgattgcctgggggcagagagagaaattaattggaaaagagcatgacagaatgttctaggataatagaaatgtatattatatatcttgttttgtgcagtggttacgtgggtgtaagaagttgtcaaaagtcatcaaacttaatgcttaagatgtgcagagttcgttgtatgttaactatagttcaatttgaaaaattaactactgagaaacattgaaatccaatgacattcctatccaaatacatatttggataggaaaaacatcttgtaaagatgtcagttctccctgttctcaaaataacaagaagttttgccttgatttttgtttttgaatttagcaaaatattcaaaagttcatctggaattatcagccaagtttaataagactgtgaagggactttacagaagctacttgggctgaatttaaaggatgagtatgattaaaatggcaataaatgtgaatatgaggtaaaagattatggagcatatttacaaaggcaaaaaactgtaaagggaaaagattgatctagtaagattagagcttggagtgtgggaaaaataagagaagagcacagttgtaatgtttgaggcttgagtcaaggctgtattgcatagttactagagatccacaatattgtcttaattgagtaatggtgtaattagatatatgtttttagatagccaattctagtgacactgtagataatgaagtgggtggggagaaattggatactttgtgctttcagggcctcttgtcttagttcatactgtctacttaacctgttgcactcttctcattctgtacctgtcagaaatcaggcccactttctcctaaaattaccacgttctgccttgtagattgttagataataaggtcttttcaacctcatttatatacttcatttatatacttgtatgagtgtttttctcctcctttcctttccatcacccatccctttgcttcagctgctagtgtcttaatagaaggtaattgcttaatgaatgcttgttgaattagagacagatcagtcctgcatatgagaactcttttagggtctaatagtgatagagaagattgaaaaaaaaaggtgatgaatttgagggtaatttaaggggtggaagtgatgtcatgcaagagaggagttgaaagcaccaaatgttttaaccttggagattgagaaagtggtgatgtcatgaagaatgtattccacatttgaataatgagcagagtaagaggtaccaacaaatgaaacaattaaaggaggccataaaaacaacagagatcatttcaacattctattgaggaaatttggggggacacactttaattaaataataacatcagtatttctttacctttgtaacttttaattaatgaagatgttttaagatgaaatataaaatgtgaacctgagaactgtttaacatttaacattatggatatacatatccatgtttagaatgttctctgtgtattaatgcatgattttgtcataaataccccactaggaagagaaaggtcggagagagtgtgtgtgtctctatgtgtgtgtatttgtgtgttttgtcctggcaaaaagacaaactgagagaacagaagaacaaaagagcagtactgaaaatagctagattaaaggttgtttttctgtcaccacacaccagatatggagagtagtaaaagcagacagatggacagttctgttagttttggagtgtgtgaatagagaaaagaaacattctgttccttttattcagaaaaggaatttctagactaaattactggtatcagcaataagtttcatattctgaccaaagaaacattattttttttttaccctagggataatgactcatgctgttctcagaattttgcttttgtggaaactaaagtgcagtcataaatacggcactgttatttagatcttagtgctaatgatactattgtgagtagactagtgatgggtaagaacactagtattataaacccagttgtaacaaagaaaccaagcacatcttaattagctgattctagatgggaaagtagattttttaaacaaattaactaattgttttaattctgtgaattatgcaatagaaataggacccataattttatttttttaatacttcattattttgaggggtttatgtcagaagttaaatttctaacttccaatattgacagtggagataattaaacatattttctctgccttttttccttatgttcctcaaaattagtataagtctactaaatcatagtcacatacaccataaagcacctatcaaaagagtatcagctaggtgaagttcaaagattatattctagttttattttaacctttaaagtcttgtaatttaagaaatagaggtttgaatgtcttctttatataccatagtactagatgctctcactctgaatttttaccatgtcaggatactccatcacatatttatctctgtaattataattattgggcacatacacaaaagagttgtcctttacggtatctctgtccaacctaaaaactttcccatttctcttcactttaatctcttcagtcgttccctttcctgaattacctctttactccctagtgacggcttaaagttttagatcttaaagttccctttttcaaactttcttacgtcttcctctgtgcctttattttaatgtcagcagaagttagctcatgaatcataacatgtgaagcagttgtttggaataattgacagcttttaatgacttatgttaaatagccagtttaaaagtttttgtcagttgataagctttataaagaagcattttaatgtattaaatcattgtaagtaatttattgtttaactgcatgccttgggtggttaactaatgtcatacagagcatcagtgaataagagtggcagtattggctgggtgctatgactcatgcctctaatcccagcactttgaaagtcagaggcagaaagatcaccagagcccaggagttcaagatcagcctgggcaacatggcgagaccccatctctgcattaaaaaaaatttagctgggcatggtagcacacacaggtagtcctagttcctagctactcgtgaagctgaggcaggagaattgcttgagcctggggttggggctgtagtgagccgtgttcgtagcactgtactccagcctggataactgagagagaccctgtctcaaaaaaaaagggtaatattctactcactgatcatgtatgttgctgcttttacacagttatacccatttcagtttgtcattctcattgcttgctttttaaggtatagtaattataatttccaagatacttggtacattgaactgtacataagtataaataaatagtaaatgcttgaaagggaaaataaagaccacttactaatatttctctcccaccattgaagtattttggctgtattgtattatcgaaaagatggattttttagaaatctgtgcttacatgacttcacattgttgttgctttcattcatagaacttgtcttcacatcttagctaggtgacacttgtgctgaaattaagctaccctctctctgattgcacttctgggagatctgagtttctttgcttgccaaagttattacttcaagctttcctgttgtgaaatagcccttttgccagagatcaaatactgaaggatgataaatcactattagtcaataattgcttgcaatggacattttcattattactaacataatactaggatattttgttattgccagaaaatggggcagatggcaaccaattacatcaggatataaagttattgtttggtctttatcatggaaaaagcattgttatttaaatcaaatttttactgtatatcatgtctttcatccaatgtcgtaacatcccagatagacttggggtgtattcacttaaaaaaatacaatatcttacgttcattagagtatagttggaagaaaatgtattatttttgcaagaagctgtttatggatttatctgattaatttatgtagagccaggaaaaagttgttttgaccaataaatttggcatttgaaatgagcaggttaagaatagacattaagtgatcaccttaagtgcaataaaatttcagtgcactatacattctgtgctttatatctatttgtaagaataccaataatatatcaatatggtataagaggaaagaataagagattactataatagctcctaagaaaggaaatcagtccaaggacataggctgaggttgtgatgtggacaaaaagataatagcaaggatagataaataactagacaaaaatgtcagggacctaagtatttatatggcaaacaaggatactcagtacatctagagccagactttttccagtgtctaatccttgttgtatattaaatactttgggaactaaaatatgttttatgacactgtgttaactgatgcataattatgtaatggaagccattagggcctgtgtcttggcaggaattaaactaaaagcaaggtccttctagtatttttggtagaagattattggctagtgcctattttcacagcacatactactaaaattaggaggacgacagaagacgagcattgcccctccctgtgcaaaattgatagacacattcaagaagtattccatatttttaagctaatctttttatttgattcacttaccagcaggctgttttcctgctgcaaagcaaattggtggagtccatttacaccttctccttagcctataagtattggcagaatcaccaccacgatcacagcagcacagtccagacagtgtcatttgattttgaaaatattgtccttgcaacagccagctaggacaatgacatactaatatctgctttgcttattatttgtgcctgggagtatgtaatcatagtcaacagacaccatagctacagttttatgacacttggtctcaggaaatagagaactctacagaagacagtagaccatgatgaagctttatcattagtccctggttctctcccctccagatcactttggccttctgacagacagtacagataaatctgttaagtttggaatgttgaattggtcaaaaatgaaaataatagtccaaagcaaaaagaccttctgtgaaacatatgtggatgcaattaggtgaatatattgtatctgctggtgtccccaaagttgatagctttatttattggaccagcctagactgaaaatcttctgtttaggcattttggtggggattttttcccctctgtggataccgactgttatatttaaggaagatagaacaattggttctaccaagaaacctgtagctaacccaagtttcactttatgatggcagcactttaaaagtttggtatgaaagactcatctcttcttcattctcaagacaatagggctaatcagtgagatgcaggcaagttttaatttatctaaatactaaagagtatcactagaagggatagtacttgaatacaagtccctatggtgactaaattatgtcctcaccccatgatataaggaaataagctagccgttgatttttgatgaagaaagactgtgggaacagtgtgtagcaactgttttccaagaagttatccactctcacccatagtagtggaaagacttgaaagaaagttgttgaggtgttcagagccacagaaaggcctattaagtcattgagctatactagaaagaaacataaaaacaaaagagcagaagccatttgataaacttcaggggggagaagttctaatccctgatgacttacagaaacatcttgccttcagcttcttacagatctttaaaagaatcaggtcaagtgatttgaaagacatattgctaatattacctttttctagattctgtagaaataaaccatttttcctctgctctcacactacaataatcaacacagaagacttctgtaactgctggtcgccaagaagtatatggggatttctccccaccaacaatcagtcagttgattctgccgcagattcttcaatagacagcagttgagtatcctgtaattcaattcagttctgacactatctgcctagagatagcatcagatcccatagattgagggctcagtcacaagactgccatccatttccaatggcagtcataagcttcaggttgttttacttgtgcttctgactgactggctataaatcagcgttcccatgaccccctccttgggtttgattaatttgatagagcagctcatagaactcagggaaatacatttctcggtttattataaagaatactacaaaggatatagatgaagaagatgcatagggcagggcttgtgggaaggggtgtggagcttctgtgccctctgtggacacaccaccttccaggaacctccacttgttcagctatccagaaggtcctcgaactcagtcctttttggatttttatggaaacttcattaagtagcatgattgattaaataattggccattggtgatcagcttaaccttcagcccctctctcctctgaggaggtgggtagggggtggaggtaggggtaggactaaaagccccagttctttaatcttgccttggtttttctggtgaccagccctcatcctgaagcaacctaggggctgccagccaccagtcaactcattagcatacagaaagacccttaccactttggagattccaaggaatttaggagctgtgtgccaggagagaggacagagactaaatatgtatttcacaatatcacatagatgttcttaatttaattaagatgttaatctagatattcttaaactcttaatgaactcaacaagctgagccctggataatgtacccttttgtttctttctttagctcacaagaagccaggaaacaactattttataagtcagccaagtccagaatggtgttagcttcattatggcaagaaagagtgaagttacttttcgtgaagccctgggctttttgaagaaaaagagaggagaggtggttttaacattgactttaagttgaaaagtagagcccttttatacttttttctttaataaatccatcaatcaagaatggaacaatgtgtggttagcatcatctttgaagttccaagtcacaaagggattttcttcctgaagtatgtgggatgtgagtcatttgtaaatgggaattacgaaggacaatgagccctattttttttggttatcatcaatataggtttggttataaacttgttgtaaagagttaaaccatcaggaccttttctcttagagttccacaggagctgaaatgtttgcttagtaggaaatcgcagtaacatttcaagaacccttccgtattttgcatttcctttatattagaccatgattccttgctaagttggtctcttcattgatctcattggttaaattttcattgaggcaaccaggtgcatagacttctattttcactgtcttcagtatatacagttggtcctgaaatggatatacagctggcaaacttaaataccttaggattgtttctctctttatgcaaaagatgctattttttagtcattttataagggaagtgttttttttttcctaatttaaaaaattaccctctatatgataaccaaatttcctttaagtgttgcttcagtgaaccaaatagtgttttactaagtgtaggagttctagctagataatatctaccatctctttacaacaagcctgaaacagtaagttctgtttccctttctggacacatgcatttagataacattgatccagtagataagagaaagggaggccgggtgcggtggctcacacctgtaatcccagcactttgggaggccgaggtgggcggatcacgaggtcaggagatcgagaccatcctggctaacacggtgaaaccccgtctctactaaaaatacaaaaaaattagctgggcgtggtggcgggcgcctgtagtcccagctactcaggaggctgaggcaggagaattgcttgaacccgggaggtggaggttgcagtgagccgagatcgcgccactgcactccagcctgggcaacagagcaagactccgcctcaaaaaaaaaaaaaaaaaaaagagagaaacggatcctttattgaatttctgtgttccaagcactatgctgagaactttacatataccatctcatttaattcttttgtttgtttttctttgggatggagtcttgctctgtcgcccagggtggagtgcagtggcaccatctcggctcactgcaacctccacctcccgggttcaagctattctcctgcctcagtcgcccgagtagctgggactacaggcgcccgccaccatgcctggctaattttttgtatttttagtagagatggggtttcaccatgttggccaggctggtctcgatctcctgaccttgtgatccgcccgcctcggcctcccaaaatgctgggattataggcatgagccaccatgcccgaccctcatttaattcttataacattactgtgagatagtaactatttttattcaccttttgcaaataaggaaactgccttcaaatgagcctacaaagaagacttaagaattggagagttctccctagggtcagtggaattcaggatgatggtgcactgatctcaactaaaacgaaactactggttggaccatttcttctcaggactagattgtgttaatacacattcttctctttgctactatatgttaaataatctggagatttccataatgtttccaggttccttggttactcatttgctacaggtgttgctaaagatgtgtaaaagctcaagtcactagttaggctctctttgtggtttgattagcttttgtccttttctttggccaggagacactctaattctgtccacctgtcttaccagtgcatgccattggtcacaaggtgggttggttaatataaaggaataaatgaattggagaccaagtattgtactactggaaaaacaaaggcaaatgttagtatatttttttccatttggaatagtagtcattcttttagttctgtattttgaaatcattatttaccaatacagtcttcttgatgtgaaaatgcaaacagagttttcagtgttacctgatttagggtcactggaactgagagtgatggtttacttctgactgcctgctgctgcttttatgtgaaactgtgataatctgccttgactcccttcctcctaggaaagaaagaaataaaataattaaataagaaaaatatttagtagaaaactctttatcactgtggaatcatttgtatcctattaaatggtatattatgaaatatatatcgttttccacattaagtacatttgccttttctcataatcctatctcaccttcacctttattaatgtttagtcacattttgagcttatattttaagaaactttaatttgtccttttgcatgctgaaatttgaatttttaaaattcaagtatgctgggcacagtggctcacacctgtaatcccaggactttgggagaatgaggtgggcagatcacttgaggtcaggagttcaagaccagcctggccaacatggcaaaaccccatctctactaaaaatacaaaaaagttagctgggcatggtagcacatgcttgtagtcccagctactcgggaggctgaggcaggagaattgctcgaacctcgtaaacagaggttgcagtgagccgaaatcatgccactgcactccagcctgcgtgacacagcaagactccatctcaaaaaaaaagtaaaaataaaaaaataaaattcaaattgaaaaagttcattttcacgattaaattcatatttacagtgctgccaaacttaaataccttaggattgtttctctcttcatgtaaaagatacataaagtgactacacatttcagtatctggtatggtttctctttataaaatacaaaatttaaagttcatgctttatctaataatctaagatccaagtattaatctatacttcattaatggttctttaccacatttgattctgctattcaaactggattcttttaacccaaccttggtcacatttactgataatcagaagtccacattgctataaatactactcacctaagccttgattatttgtcattaaaaaaaaaatgtgttagcacagatcctctctaaacccattcatgggcctcatattaggaaacccttgtcttatataatgcattaaatctctgaaatatatttttataacatgtttcttagcactgtaaatatgaatttgataatgaaaatgaattgcagttcctcactaaaggaatttaaattttttgcatactagtgatgaggacttgattttccaccctgaagttgtaaaggtctttcagtctctctaaaagtgtgagttaaatctgttttctcacggccagcttttaaaggtcagtgaaggacggcactttgaaagaaatggataatatgcatccttccagagctagtatatattacagctgtccctttggactcactccagaaacagctcagtgactcttagaggaagagaattttagaggccctaactggggcccagaaaattgtactgaaggatacttgcttcctgtgcatctccactatagtaaaatttgtttaaaattacgcttcttagccctcaaatccaaatggtctcttgagtgcttttgatttttctttaaccatccgtttcctactatttctactgctacctcgctttgttcaggcccttgttacattttcactagagtaatttcaacaatttagtttttctacctgctcttctatgctctaggattatagtgcataaaaaaaagtgcactatatatatagtgcaccccccccaccacccccagcataaaagaccctatcctgaaaaagttcatagcctaataggaggtcaaacatgactacagataattttaatgagtatgttgaggtacagttatagaaatacatataaaaggctgtggtaacatttaaaaagggagtaactgactctagaaatatttaaagtaactttcaactgcagtaataagaccattgaattattaacccagatttgaatcatgttgtttctgtagcaaatgtagagggaagttttgttctcctgttccaaatcagactaaagattcatttggtaatttaaaaatcagaaaagctttttttttctctctctctctcgttaatgggttgtaggggggtgaaagaaccgtctctgtctcctctggaggctcagtatttgagtccaagaaataaacttgacagtagacagattaacaggagaaaaggcatgtaaatgtatcgtatgcatatgcataggggtcctacaaagtatgagactcagataagggccagacggctgaagcttaaatagtacttttagctacagaaacaaatatgggcttaaggctcctggagggtgaggggaagaactgtactgtgaacgaaaattgtcttgctctgcggataaagtctctcagatagtagccctcagaagaataggtgaaaagtctgtatgggcctggtgtcttgggtgtgcggacctctagtctcctctcctgtgataggagttaatgtaaattccagagatggggttcacaattccattccttctggaggaacttccgtcttgataagggaaacttcagagaaagccctttcttcacttgaggagagaagatgaagagacagaggtacaggggaatgtcagaccttggttctgatgctgcttttttagttcaaagaactcatcatgtcaaagtgtcatactttggggtatagtttcctgacccctaacatgagcaaataaagagatgtaactgagttagccacaaaactatttttttctgttggctctatccaaaaatgagagtggaaaatgttctgtgaacatatatctaaaagtatgggtaaaacttgttttacaaactgttggtcattcagcaaacatttaaacagcttctatacttgtggaacataaataactagaaaatacaagctgcttattttagcaaattgtggctgtttgcattgctcaggctgccagttgtgccaattgtgctgaggtatgcttcaaaataactttctttcttatttttaatgactttttaatgcaggtacaatatcgatgttttaaaaacataagtatttggtgttttgttttgttttgttttgttttttgagatggagtttcgctcttgttgcccaggctagagtgcaatggcgcagtctcggctcactgcaacctctgcctcccaggttcaaacaattctcttgcctcagcctcccaagtagctgggattacaggctcccgccaccagggcccagctaatttttgtatttttagtagagacggggtttcaccatgttagccaggctggtctcaaactcttgacctcaggtgatctgccctcctcagcctcccaaagtgctaggattacaggcatgagccaccacgcctggcctaaaaacataagtattttaaacataatacacttttgaaattgaaacctgaactgaaatatataaaatatactttctgcatattttaaagtagttaagccagaaaacatcttaggctgtatttatagagactggtaatccatagtcaagtaatagttttctgtactctgccttggaaatactgattccagaatattgtagtagattttcattgccacattttgagatgaatattagcaaaagggcatatccaatgatggcaaaaatgagatggtaaactatcttgcctgaattatggttgaaggaattggtggaaaaagacccaggggaaattctaatagctttcaagtatgtgatgagatagagaaagtggaactgagggaatgaacaagataatttaggagtaactatgtttagggataacttcagaattactccacaaaaagttccctgtcaggcagtaagctcccagcaatgaaagtacccacttaaatcacttctgagatactttcctatttcaagattttaacttaagggcttaaagaacttaggttgacactgtataggactgtagtttgcagaggcagggtttgggataacttcttagccactgggcacttatgcggttgataccaggtaattctaatacattacagggatcagcaaactttttttctgcaagatgccagatagtaatattttaggctttgcaggccatatggtctctatagcatctgctcaactctgttgttgcagtgcaaaagtattgttgacattatgtaaacacagtttgttacctgtgttccaataaaactttatttacaaaagcaagtggtgagttggatttggcctgtgggtcatgctttgacgacccttgccttaactaatgaaatctagaaatagagttctaatgtaaatatagaaaggctttcaatctagggattaaaaatcgaactgtcaagatgtcattgtcttcttggtctgaaagagtacacatcaagtgttcaaaatagtgaattgcccaatgaattaagaaaccttccagtataaaagtcaaatgacaatggagaatctgatactgttttttactctttatttctgctacttaacgttgttcttaaaggcgtcgttacttctgctgggtagtactgtctactcagttggactgctgatgcagacccagtccataccaacaagttaacacaggttcaggccttctgagaaccaaaagatttttaagtacttccttcctaaatacagtaacttttaacagtaattttaacaccagaagcaagaagtaaacaaaaatctctgatgatcgaggcatctgttgtcatatattttgtgctcctaaggtcatacagcagagcctcttgtattatcttttggtctgttggcctttagtttatagtttgtaaaatggtggtcacatgtcaatgctgactagaagaaggagatgctactgtaatcaggcaggttggccttcgctgagaagtgaccattgattgctagaaaacaaagaaatgggaggcattcagcaacctggggttgttggttttaggaccagaaatgtacccccaagtctccaccaaatttctcaataatcctgaagagttttgctgtttgtgttttaatataacacagtaattggtgcttgtaatgaggactataattcatcaaaaaggtaaattgtgaattgtgcacacgcacacacactcttctaagcaaatgggactgctctattttgaacagtcttcaaattgaaataacttatttcgatatgtagaaaattaggataagcaaaaccaatttatttactcttaagagttccagatagtagtgatttgtcagtatgcatatgaatctttagtcctggcattctgaactcaaaacctcaatagagtggtttcttgacccttaaagtgttttatttttagtaaatacatttttaattctgtgacctcaaacctccaagctacattatgatttcataattttatagtgatttatccccccattgctttaaatttaataatcccatcctctttattggtatttaattttttctgtagtctaaaggcccttttatttaaaatgttatatattctgatttcttaattttagggatttttctctagacaattgagaatatgtaatttttacactttaatgttcatttgattataacctacacttgaaaagcaatttttaaagaaatctgagagaatttaaaaatcattttaaaagcatcatgtcatttttagctcaaaatacttcagagaatttattttattttattttttttggagacagggtcttgctctgttgcccaggctggaatgcagtggtgccatcacggcttgctgcagccttgacctcccaggctaaaatgatcctcccacctcagcctcctgagtacctgggaccataggcatgtgccaccacacctagctaatttgtttgtttttttgtagagatggggtcttgagcccaggctggtctcgaactcctcagctcaagggatcctgctgcctcagcctcccaaagtgctgggattacaggtgtgagccacaacccccagcccagagtctcctttttactaacggtgtacaacataaaattcaaactctaacatggttttacatctggcccctacctaccttttgcctctccttctttgtcacatactccacctgttcccatgcactttagactcaagtaatgaaaaacagcttgtattgctaggaacaggccgcattctttcatggtctgactttatacacactctcttgtggcaacctagaacacctgtctataggctctcttacatcaggccataccctttctgtgaaatttttaatctgtaatattctactgaaactttacctctctgaagccttccctaatctcctcaggcttttccattgttcatattctgtcttgtaacagttatcacattgttaaagtcgttccccagtaaactatgagacccttaactgtggaagggaaggcatctttttaacctttgtttttgtgccatacttagcctagagcctgacattaagcaggtacttctcaaaaaaaaaaaaaaaaaacacagggtagagacagataacaagggattcctgacacctacgctgtagatagctataacatttcaataggaatcttgggaatctggggaactggaagcagctgggaaagcaccggcatctgagccactcagtactcttccaggcttgtgtgtgagccttgccaccttcaggtattagcacttgaaatctaacttctttatgaagctccttatttacttgccttctcggtgaaaaaaaaaaacaacaacaaaaaccgtttccttccccatctggtgccagcactgtataattcctaataagcttgaaaagataatgttggcaatactttacaagtttattgctaatgtttaacatttattaggtgcttgctgtgtgctgatctctgtgcttacatatttttcatatatcacttcatctatcttcacaacagtccagtgaagtagatgccattattatcccaatttcacaaatgggaaaaatgagattcattgagataaattaaactacccaaggccatacaactatagactaagggggccaaaatttgaatctagtgtagattcaaattagagctcattctcttattcactactggggaatattgcctggccataactgaagtccaaataacagcatggttataatatgggctttaaaataatctagacctggattcaaaatccatttattggaggcaagtgacttcacttctgatcttcagttttctcatatgtaaaatgataatatctacctcaaaagtgttactgcccacccatgtacatggtacatactaaaaaagcaaatgttagttccatttgccacttctacactttatgtgaaccctgggggtaatggtttgtacagaataaggcctggcatgagaagacaaaaaccatctgacaaataaaaaagctgtcattctgtctaccagaatgtcaaccagtgcctgccttccaggatcaacaagtgttttattaccataatcacatttattctcttaatgaaaagggtgtgtcttgagtatttaatagttaattacccataaatatactacagtagaataaggaataaaaccagtgatctgtctactatagtaactaggtttctttcttggtaatacttagggaacatatgagggttccctgtgggttgttatttattatataaaatagttgctctggtagttccataatagagacctgtcagtgccattcagcagagaaaaatcataaacacagctgctgtttttgtgtggcttgtggttttggttttgttttcatttgaacttttttatgcaggcatgtcatcatttaaaagtagtcatttggcgttttcccttgcattcttcagattttttgtaggcttaaaaaaatttaagtcacttctataattgttcatacagattgatccatctggtctttttactagccaatgaaatttttgacatgtaggaaaaaggacaattattttaaattctataatttcagtggatctcttttataagtttgaagaaaaatagtttcaactatcaaaaaatatttttcttatattcttcattgtttcctgttaatttctgtattttcgtgggggctaaaaaggctaataatttctgctattcctactcttcacatcctttgtgtgttataactctgaagcataccataattatttgtttgcaaccaagaattggtacagcataaatgtctaatcaaattaggccctcttgctctctttaagtcacactggctatgaggtggaggtggattatagggggcaggcatcaattcagggagaccatttaggcctttgcagtggtccagacaagaaatattgatggccggcccaaggtactaagagtggtatttgaagaatgtagacagaataaagagagaattaacgaagcttgttgatgggttagatgttgattggtgtgagatggagagaggagtcagggatggcaggtttcctctcttctttcttgttgtgtttgttcaactcttgcttatcttttttttttctttgttttttgatggagtctcgctctgtcgcccagactggagtgcagtggagcgatctcggctcactgcaacctctgcctcccaggtttaagtgattctcctgcttcagcctcctgagtaggtgggattacaggtgcatgccaccacgcccagctaacttttgtatttttagtagagacggggtttcaccgtgttggtcaggctggtctcaaactcctgacctcgtgatctgcccacctcagcctcccaaaatgctaggattacaggtgtgagccaccatgcccagccacttatctttaaaggattaagtttatgtttcctactatgggaaaccatcccaccccaaacttgatgaccgcattatgtgcttttatagaacctggcacttctccaggatagcatttattctgttttgtaagtgtgaatgtaattaccctacacacagcatacacataatcttcatattctttgccttgtcttgtgaaggcaagggccatgtctatcttattcgtcattagattcccacatccaacatagtcctggggacagcaccaatgcacttttggtgcataagcaaatagtgtcatttatagctcttacctacaatatctgatagactaatcaaatatagtaggttatctgggcctttttgattcatgtctctagcttaactttcatttttttcttatttggtatctctcactttgccttttgatatactcttacagtttcgctcactgagtaaaagaaaatataaacagcaagaagtaaacttgtgttttatggattttgataacatcttctaaaagaccccccaagattgttgatgtctaaaaaaattaagggccttcaactcataataatacttaatagttcttaaaatattacaaactgattggaacattgcactaacagagagagtcatggatcttatttatgtgtagattcatttcagtcagaagtattaggactggtttatttgagaaagtaaatagatcattacaaattcatcgtcatacttcaaggaaaattcattgatgacactttctaatattgtgactatatacctaaatcatactttaagaacagaatgtctaagatatcacctagataagaatcaatacaaaaagaaaaaggaaaattacaaagattgatttaagaaagacaaaagactaagccataaaatttctcattattacaggttgagggcagggagtgaaaaaggaaaaggatttgacttctctaggcatggttttacttattttaaaataagagcagtggacccatgatctgaaagattgtttctggcctaaattctgtttttccgtaagatagaagagaatgataaaggtgatatgcaatgggaattttgggttgaattgcaacttacttttcattcttcccatagcctcatctctgtcttgatgaaaccttagagaatccactcaaatatagtgttttcttaaatgactttattaaatttaattaaatacaaatggtttttctaatctctgctgaaataaattctttgtagcctaatttagagcaacagtctagattagaatacttaagatgaattattactacttagcctaatcaagtcaaactatggaaaatgagtttttacattttagcagtgttataaatgtattcttctgtagttgtgtctcttttaacatatgttttccttctttgatttaggtttctgctttgggacaaccatacatctaattccttaaagtagttttatatgtaaaacttgcaaagaatcagaacaatgcctccacgaccatcatcaggtgaactgtggggcatccacttgatgcccccaagaatcctagtagaatgtttactaccaaatggaatgatagtgactttagaatgcctccgtgaggctacattaataaccataaagcatgaactatttaaagaagcaagaaaataccccctccatcaacttcttcaagatgaatcttcttacattttcgtaagtgttactcaagaagcagaaagggaagaattttttgatgaaacaagacgactttgtgaccttcggctttttcaaccctttttaaaagtaattgaaccagtaggcaaccgtgaagaaaagatcctcaatcgagaaattggtatgatacaatatcctattctaaaatgcaaataaccataaagcttaactgttgtccctttctaaaatatttctgtctaaaccaataccttcgtaatcttaaatagctttctaaataaaaatcataaatctaaagtatgttttactatcgaactatggaactatttttaacaccttgatattattccataaggttttatttaagaaatgtcatttgtgggatgacttagatttgttatatctcagtgttgttattcttttaaaaatgattgataggaatgtttgctgcctttgctctaaattgctgaatatattattttttatatattaaaaatattcaggactgtcaacttttaatatatatgcattcatcaaaaatttgttttaacctagcggtactttttttactttattgtgatcttccaaatctacagagttccctgtttgcaaaaaaaacatgttcatgctgtgtatgtaatagaatgttatattctttatgtaattttattaaaggttttgctatcggcatgccagtgtgtgaatttgatatggttaaagatccagaagtacaggacttccgaagaaatattctgaacgtttgtaaagaagctgtggatcttagggacctcaattcacctcatagtagagcaatgtatgtctatcctccaaatgtagaatcttcaccagaattgccaaagcacatatataataaattagataaaggtaagaaaatgactaatctactctaatcattactatagtgcagtcttctacctgtgtctatatctttgtatagtctttttttttttttccagctagatagtaagcttcttgaaggcagggactgcttatactgacaagatatagttgagtgctaaatagaaattatgtacaatcaatatttattggttgaatttttgtgtgaatatgatactgatttcttggtaaattgtctataaaacggaagaagtatgagtttgaaatttactattttttaggattcagaattagagctgattatcttttcataaataaaaactataaatagaaacattttatgaaattttgggaaaactttatacattcttgttattatatatcataaatacaccagaaaaaaataagtaattttcttaagtattaaatgcagtaattctgatcatgggtgataatatatagggagatttttgatatttaatatattagtttctagaattaagggaacaagaaactaaactatattgttttcaaaatgcatgtcattgtatcagataaatatctatatttttcagggtaacagtaaaattatcaatttgagttaactctcacacactattaaatatcaaattctgttagtagaataagttaagacatcttattactattcgtatttttcaaagtagtttaatgcaacataagatctttcaaaattcttttctattctagcatatattttaatgctcttttcattttcccgaacattctttgtgaaaaattttcaacatatagacaacttgaaagaaatgtactgtgaacaaccaaacctctctaaatagtttgtcttcccgttttcttcatctttctcttttcttcttttttggactcctggcttaatcttaaaaggagaaaattagacaatgcatttcctctcccctcgtaattttcttacctttcctccctcacattttctgtcattctaattgaacatctttaaacctccattggttctttctttacttcccatagttaccaaaaacttccaccttaggcactgtcaaacctttgagaccatcatgaatcactattcataggggcagtacccatgaagtatgtcatacagtttagaatggaaattagcttggtctcaggtacttgtgacccacatatcacagttttgtgtgcttgtcataaaacatgagatttgggaatgatctggcagcccgctcagatataaacattttctgtttctacctgatatttacctagttttaattgggctgattaaaaagcatttctgatatggataaagtaatgatagtgaatacttgttgaaatttctcccttgaaaaatgaaagagagatggtgattgcatctaatgttttcctgttatagggcaaataatagtggtgatctgggtaatagtttctccaaataatgacaagcagaagtatactctgaaaatcaaccatgactgtgtaccagaacaagtaattgctgaagcaatcaggaaaaaaactcgaagtatgttgctatcctctgaacaactaaaactctgtgttttagaatatcagggcaagtatattttaaaagtgtgtggatgtgatgaatacttcctagaaaaatatcctctgagtcagtataaggtgagtaacaagtttcaaaatattaatttttaatttaaaaagtaatcacattgaggatgagtatctgtattttttttttttttttgagacggaatctcactctcgcccaggctggagtgcagtggcgcgatctcggctcactgcaggctctgcctcctgggttcatgccattctcctgcctcagcctcccgagcagctgggactacaggcgcccgccaccacacctggctaattttttgtatttttagtagagacaggttttgcaccatgttatctaggatagtcttgatctcctgacctcgtgatccactctccgtggcttcccaaagtgctgggattacagacgtgagccaccacgcccagccaagtatctatttttaggatatacttcttgataagtaatattagtaaatagcgtttgtaagcttattcttttaattctgtgattaattcgagaggctgaaaatgttggcagttactccagctcccaaatatagatattccatggggttgttgtttttgttgttgtttgtttgtttttcgagacagggtctcgctttgtctctcaggctggaatgcagtggtatgatcatggcttactgcagcatcagcctcccaggctcaagcagtcctctcacctcagcctcctaagtggctgggaccacatgcgtgtgccaccatgcccagctaatttttgtttgtttgtttgtttgtttagagacagagtctcatattgtccaggttggtctcgaattctgggcacaaacaatcctcctgccttggcctcccacagtgctgggattacaggcgtgagccattgcgcccagcctatttcgtgatttttatcccctccaaccagtggctgtggaatggaattgaaatagacatttcaaaaaatccatgacctactgttaagatatactattgatactttcctactctctttttctgtttattaaataaatacctataagaagcaactacgtttgtgccaagcatatgtcgagtactaatttacataaccaagtaaaacctgggcctggccttcaggttgcttattttctagtggggcttatgggtaagaaaataatgtgtatatagtaacatttatattaaaaatctcgtcttaactaccatttcaaaattcagaccagtatattttaacattttttgtaaatatccataatgttactggtctccactatttgtggttttttgttttaattttagcaaaactaatgtttctcaggaaatgtttggacaaaaaagcaagtgaacagcagcctttggatgggacatcagtatgacttaatagattgaatgacagaggcgtcctaagtgatattacttttctgttatcatatacaatgttttcataatgagtatcctgtgatccttgtttttttttttttttttttgagacggagtcttgctctgtcgcccaggctggagtgcagtggcaggatctcggctcactgcaagctccgcctcccgggttcacgccattctcctgcctcagcctcccaagtagctgggactacaggcgcccgccactacgcccggctaattttttgtatttttagtagagacggggtttcaccgttttagccaggatggtctcgatctcctgacctcgtgatccgcccgcctcggcctcccaaagtgctgggattacaggcgtgagccaccacgcccggccgtgatccttgtttttaattcctttttttgcctccagttaagggtagaactacagtttcaaaagttgaccttaatttttttctttcgtgcaatttatattcagaagtgtttgattgatcttgtgcttcaacgtaaatcctaaatgttagtattttaaatgttataggaactactagtaaatgtggtctataatgtttaattttttatcacctttgcagattaatatgtagtcataatactctgacatgttacttttaaaatgaaaaaccttacaggaaatggctcgcccccttaatctcttacagtatataagaagctgtataatgcttgggaggatgcccaatttgatgttgatggctaaagaaagcctttattctcaactgccaatggactgttttacaatgccatcttattccagacgcatttccacagctacaccatatatgaatggagaaacatctacaaaatccctttgggttataaatagtgcactcagaataaaaattctttgtgcaacctacgtgaatgtaaatattcgagacattgataaggtaaagtcaaatgctgatgcttattattttatagaaattattttagataacctttttcttgcactatacagtaatctgttgacctgtagtatgttttcagatggttaggagaacatccaaatctccgaatgtaaaaatatatcaagaattttacttgagcttccatctaccttagctattatacagctcacagtcctttgttaataattctaatattcacaattctagctcttaaaatcaaaagttttacagaattcgtttggcagaaagacctgggccaaccttaagtgagggtttttataatctttattaaccccacttagtataaaattccggtatcttattaaagaaatattaatgtctttatgaggtactgcttcaccagctaaggaagtagtatttagtaagtacgtgtaccaatttagctttctaaaatatggaaaaactctgaattacatacctcccttaaggggattgtgggcctatatttatgttttagtagtctgatgtctccattgttattagtggatgaaggcagcaactaattttggtgaagactctacatcagtattaacgtgttacatatgtgaaaaaaaggagaaccaagctatatctgaacaaaaattccgtggttttatatttgagtctatcgagtgtgtgcatatgtgtatgttgagtgtatacattagtatatacctacttttttcttttagatctatgttcgaacaggtatctaccatggaggagaacccttatgtgacaatgtgaacactcaaagagtaccttgttccaatcccaggtaaggaagtatatagatttatatttccaaaggttatattagtgtttagcagtatgatccataaaagtagtatatttttttagaccagcctgggcaacaaagcaagaccccatttctacaaaaagtttttcttaaaaattagctaggcactggccaggcacggtggctcatgcctgtaatcccagcgctttgggaggctgaggcgggtggatcacgaagtcggagtttgagaccagtctggccaagatggagaaaccccatctctactaaaaataaaaaaacattagccgggcgtggtggcgggcacctgtaatcccagctacttgggagactgaggcaggagaatcgcttgaacctggaaggctgaggttgcagtgagctgagattgtgccattgcactccagcctgggcaacagagcgagactccgtctcaaaaaaaaaaaaaaaaaaaaaaaaaaaaaaaaaaaattagctaggcacagtggcatgtgcctgtagtcccagccattagggaggctgagacaggagaataggagaattgcttgatcctggaagttccaggctgcagtcagctatgatcgtgccactgcattccagcattagtgagacctgatctcaaaatatatatatattttacatcattagctttatacaagctatatattaataaattaccagaaatgtacatatagaccagcaagactttctgtcaaatgagtatcagtcacaggattattttattgattctgtctaaagttttatcagaatagtttctgattgtctcttttcattgtatcaaagagagaacagagtatggttataattttatgctaatcattccccacattagattaaaaacctagcattttattcttttcctgtctccatcatcattcacatgaaggagaagtaagagttggcaagcaactccataaccaaactaccctgaggcagtttccttcaaatcactcccaagaatagatatttatggctttcaaataaagggtctttggtagcaaattaatttgttagtgggagagggagttatggggaacattttaaattattactgcctggatgtgatgaagaaataaaaagtagtataatccaatagctgactatcctgcatttctttgcttctctctatacagtcatttgggaataagagtacccctgaacgtaatagcagtaatatttcaagagaactcaaattgttattggactttatgataaaataacaatagagctaactagtgactcctccagaagtaaatctcataatgaatttagactgagttctaagacattcaagaaaaagttctggtcttgttgctaaagatggccacccttttagagcctaggtaaggttaaaagagatggtagccttgtctgccacagggaaggaagatagttcctcagcctacctaaaccactgtaggtcagaaaacgtatcattcaggtcagaaatgagattgtgacaacaaacaacttcaggatctttttttttttttttttttttttgagttgaagtctcaccctgtggcccaggctggagtacagtcaagtgtaagcgattctcctgcctcagcctccctagtagctgggactacaggcacatgccactgcgcccagcttattttttgtattttcagtagaggggggtttcaccatgttggccaggctggtcttgaacttctgacctcaggtgatcccacccaccttggcctcccaaagtgctgggattacaggcctgagccatcatgcccggccaggatctttttaaattcttgctaaaacaagagtttatatcccactcatactggtatgcctcacactgattagtggggtggttgtagcaggaaaagaaaaacaacatttgaggaaataatagccagaatttttccaaatttggtaaaaactataaacccgtcaatccaaggagttcaacaagccccaagcaaagaccccaaaataatactggatagaattatcctagctagctgtttcaagtaccacaagttaaactagctttataaaagtaaaaggctgggcgtggtggctcacgcctataatcccaacactttgggaagccaaggcagatggatcatttgagcccaggagtttgagaccaccctgggctatatggaaaaacacagtctctaccaaaaatacaaaaaattagcctgggatggtggtgtgtgtacctatagtcccagctactcaggagggtgaggtgggaggatcacctgagcccagggaggttgagactgcagtgagccatgatcgtactactgcagtccagtctagggggcagagtgagaccccgactcaaaaaaaaaaaaaaaagtaaaagataaacttatttaataatatatgattaaaatgttgaaatgttagtaaaatagctatttagaaaggactatatttgtctttaagcattctgtagtctaaaaggtgaagtatttatggtagtgtgaaaatttaagtaaactttttaaacttttctgtgcttacaaaaacaaaaatcagataaatgaatatgaagttattaaaaagatagaaaagtagattttaaaaattcttactaaatatttgtttcaaatctgtgcttcttttatgctaaattagatgctttatgatttctttgtaaatgttacaagtatctcccaaaatcagagtcaatttggactgtatacacacacacaaccattttctagaaataaagaacttaattgagcatgtgtatatgtgtgtgtgtctgtgtgttgtgggatggatggactttagaagaaaagagaaacatgctttattttgttagcatagggctgaacaagtagatttgtaggacatagctctcttagagtatagaacacttcttattaatttatgagcacttctaaagatttaggaataataaaaagtctggtacattaaagtaatatatctttaatttttcttttcttttttttttttttggaggcggagtcttgctctgtcgcccaggctggagtgcatggcacaaactcgactcactgcaacctctgcctcagcctcctgagtagctggagttacaggcgcccaccaccacgcccagctaatcttggcatttttagtagagatggggttttgccacgttggccagactggcttcgaactcctgacctcaggtgatccgtccaccttggcctcccaaagtgctgggattacaggtgtgagccaccatgcccagcctaattttttttaaatcattgcggacttaagtcccataatatttttctctatatgtttctacaattatatgtgttactgggcatggtagtatgcgcctgaaatcccagcaactcaggaggctaagggaggctgaggcaagaggatcacttgagcccaggagttcgaagctataatgcactttggtcatgcctgtgactagccactgcacttcagcctgggcacatagcaataccctatctctaaaaaataataataataataacatttgttttttcttttacctttagaattcttgccacatttccccattttaaatgtgtcttgtttctataaatagaccatatttttaaaaatctttatccatacttgagcaaaaatgctagaataaatggaacttattttactgatgaagtttattacttagttggctaattaaaaatagaattgttatactattttattgttgattaagaattattttatttataatcaagattagaccagaatgataagacatttgaaaagtttttaaaattactatctaacatatgagcttctatcatgggacaattgctattctaattcctttgcttgtgttatctcatttaattttatgagatagttatcaaccttatgagggtaggtactattattagtcccatttcatagaggaggcaaaagacaaagcaagggtcacacacttagtgaaaaaggggaaataagattctactccagtctgatttctgagcccatactctttatccattgttataatgtttttttaaaatacaaaactgtttattgtatgtggttctttacaaatatgttcaccaaagacagacataatttcacatgtagcacccagtaaattgagggaatttatcattaaaattcaaataattttttatcttgttcactttttgtagcagtgttttattcttcccttatgaatgtgaagtttcattaccttttataaaataattattcttcaattcaaatactttatgtatccatcattttatttacctactgatttatttagtggattcctttggataaaactatattatgtataaaagtaactaaatgtaatcataataggaaatcttattttacctttagttttccttttgtttgacttttatgagcaaaattatatatataaatatatagaaataataaatatatatttatattatgtatatagtatattatataatatatattataaatataaataagttttttagtgacgtatgtagcacacatgcataaaagtatacaaatcataattgtatagttcagtcacaaagtaaatacccatgtcttctgcttttaacaaatgcacacatttctgtggttttattcctacaattggagctgggtcacagggaatacatatgctcacttttattagattatgctttccaaattggttgtaacaaattataccccaatactcttagtgtatgagatttcctattactgcataaccttaccaccccttggcatcatcagtctttatagtttagccattcttatgtatagtgatatctccatgagattttaattttcattttctgtatgactaataatgactcccttttcatgtttattaactatttgaatatgaccttataaagtgccttttccaatcaatctctttcctgtttttcgtttggttgatctttgtcttcgtgatttgtaggagtcatttatatactttgatgaagacttttcttgatgtattatttttgctttaaaattttacataggtggaatgaatggctgaattatgatatatacattcctgatcttcctcgtgctgctcgactttgcctttccatttgctctgttaaaggccgaaagggtgctaaagaggtaaagtatttcagaaggaacaattatgtttacctttaaaaactcctgattataccgctgattgaattttttcacaaattggatgttattttatatttaagaaaataataataaacctatttttaaaattttaataaatgtatcatggaagaataccttgggagagcttcaggaatttatgatgaatatgttttgagttcttattgataccatttttaaaaatgcaaagtgactatataacagggattgcatgcaaatatctcatgcttgctttggttcatattttctatttataattaaaatacatgtaatttcaaatggggaaaaaggaaagaatgggcttaaaccttgaaaaatcaattttttttttttagatattcccattattatagagatgattgttgaattttccttttggggaagaaaagtgttttgaaatgtgttttataatttagactagtgaatatttttctttgttttttaaggaacactgtccattggcatggggaaatataaacttgtttgattacacagacactctagtatctggaaaaatggctttgaatctttggccagtacctcatggattagaagatttgctgaaccctattggtgttactggatcaaatccaaataaagtaaggtttttattgtcataaattagatattttttatggcagtcaaaccttctctcttatgtatatataatagcttttcttccatctcttaggaaactccatgcttagagttggagtttgactggttcagcagtgtggtaaagttcccagatatgtcagtgattgaagagcatgccaattggtctgtatcccgagaagcaggatttagctattcccacgcaggactggtaaggcaaatcactgagtttattaagtatcaattataatctgtggatttaggtagatactttctctatggaaaaggatccatatattttactggcatagatactatgaactctaggaccaatattgcaatagaattaagttctctatatgctaatatttttactgcacctctgcttttttaaagccctagcttgccttcattcatttaacaaatatttattgaatgccgactatgtgtaaagcactgggaaaggcactaggtaataaagtcatgaactaaacagaccttcattgagtatgcactctaactgtgaaaacagacattaagcagataaaagcagaaatataagattacagttgacccttgaacaacatggatatgaactacactggtctacttatatgtggatttttttcaataaatatattggaaaattttttggagatttgtgacaatttgaaaaaaacttggagataaactacattgtccagaagtatcaaaaaaattgaagttaggtatgtcatgaatccataaaatatgtatagatacaagtctattttatcatttacgaccataaaatatacacaaatctattataaaaagttaaaatttatcaaaacatacacacacaaacacagaccatacatggtaccactcatagtcaagagaaatgcaaacatatataaagatgcagtattaaattgtatctgcataaaattaactgtagtacatactgtactactgtaataatttcatagccacctcctgttgctattgtagtaagctcaagcattgcatatatccacttaaaatgctatgtaggctgggtgcagtggctcatgcctgtaatcccagcactttgggaggccgaagcgggtgaatcacctgaggtcagagttcaaaaccagcctggcaaacatggtgaaatcccgtctctactaaaaatacaaaaagccgggtgtggtggcatgtgcctgtaatcccagctactcgggaggctaagttaggagaattgcttgaacccgggaggcagaggttgcagtgagccgaggtcacgccattgcactccagcctgggcaacagagcaagactccgtctcaaaaaacaaaacaaaacaaaatgctatgtaatgctgatcatctctacatgagtacttcatcacttcagtaaattgtttattgcagtgaaaagtgatctctcacagttcttggatatttttcatcatgtttagtgcaataccgtgaaccctgaataacactatgagggctgtaaggagtgcctctagtgatgctagaggtactctcaagaagcagagaaacgtcatgacgttacaagcaaaagttgaattgcttgatgtgtaccatatattgaggtctgcagctgcagttgcccttatttcagacagacaactaatcttgtaaacagacaacataaactttgtttgtttttgtttttttgagatggcgttttgctcttgttgctcaggctagagtgcaatggcacaatcttggctcactgcaacctctgcctcccgggttcaagtgatcctcctgcctcagcctcctgagtagctgggattacaggcatgcgccaccatgcccagctaatttttgtatttttaatagagacggggtgtctccatgttggtcaggctggtcttgaactcctgacttcatgtgatccacccgcctcggcctcccaaagtgctgggattacaggcgtgagccaccgcgcccggccaaactttttttttttaataagagatggggtaggtggatcacacctgtaatcctagcattttgggaggccgaggcgggtggatcacgaggtcaggagttcaagaccagcccggccaatatggtgaaaccccgtctctactaaaaataccaaaattagccaggcgttgttgtacatgcctgttagctactcaggaggctgaggcaggagaatcacttgaacctgggagatggaggttgcagtgagccgagattgcgccgctgcactccagcctgggcaacagagtgagactccgtctcaaaaaaacaaaaaacaagagagagatggggtctcactgtgttgcctgggctggtctcgaactcctgagctcaagtgatcctccctcccctacctcccaaagtgctaggattacaggtgtgagccatcatgcccggccaaaagacaaaataaatgtatagtatcagtaaatacagtacagtactgtaaatgaattttctcttccttatgattttcttaataacattttattttcactgtcttactctattgtaagaatataatgtatagtacatataccaagtaagtgttaatcaactgtttatatttctgttaagacttccaatctacagtagtcatttgtagttaagtttttggggagtcaaaatttacacatggattttcagttgtacaggagatcagttgcccctatcccctatattgtccaagggtcagctgtataaatcacattaagtgccaggaaggaaaaaacaatagtgagctaagaataactaactgaggatttgtggggaaaaggcaaatatatatatattgggttcaggaaagacctctctgagatgataaccttgaagttgagactgaagtacagctatacctctgaggtattgtgagtggtctagttctagaccactgtactaaagcagaaatcacaatagagtcacaaatgttttggtttcccagtgcatataaaagttctgttcacattatactgtcatctgtaaagtgtgtaatagtctgtcatctgtaaagtgtgtgatagccttacgtctaacacaacaatgtacatactttaaaaatattgctcaaaaatgctgatgatcatgcaaggcttcagcaagttgtaatctttttgctggtggagggtcttgcctcagtgttgatggctgctgacttaccaagctggtggttgctgaagtaacgttagcgtggctacagcagtttctttaagacgacagtgaagtttgccacattgattgacccttccttatgtgaaagatttctctgcagcatgtgatgctatttgatagcattttgcccacaatagaacttctttcagatttggagtcagtcctctcaaaacctgacgttacagttaaccagctgcattagcccctaacaagagagtcagcctgaagctttgaagccaggcattaacttctcttctctagctgtgaaagtcctagatggcatcttcttccattataagactgtttagtttacattgaaaatctgttgcttagtgtagccaccttcatcagttatcttggctagatcttctggataacttgcagcagcttctccatcagcacttgctgcttcacagcttcttttttaaacctcttataccaacctctgctagcttcaaacttttcctctgaagcatcttcgtctctctcaatattcatagaattgaaaagagttacggccttgctctggattaggctttgacttaaggaaaagttgtggctggtttgatcttctatctagaccactaaaactttctccatttcagcattaaggctgttttgttttcttatacttcattcactgaagtagcacttttaattttcttcaggaacttttcctttgcattcacaacttagctgtttggcaccagaggcctagccttggcctgtatggcttttgacatgcatttctcactaaacttaacatttctagcttttgatgtgaagcaagagacgtggcactcttcctgtcacttgaacacttagaggccattgtagggttattaattggcctaatttcaatattgttgtgtctcagggaatagggagtcctgagaggaggaagaaagaggagcaaaggttggtcggtgaagaagtcagaacatgcaacacttatcgaataagttcaccatcttaattggctcatggcacccccaactgcatcagagtaacatcaaagatcactgatctttgatcacagatcatcataatatataataataatgaaaagcttgaaatattataagaattaccaaaatgtgacacagagacaaaaaatgagccatgcatttggaaaaatagtgccaggagacttcctttacatagggataccaccacaaaccttcagtttgttggaaatgcaatatctgtgaggtatgataaagcaaagcacaataatacagggtacacctgcacaaatatattgttagttgagcttggaagcagagaaaacagcaggtcttcaccatgttatcccaggttccccaggaaagagcgtatgtaagatggaatttaaatattgatctagttgtctaatcttggccatagcttttgaaccacagtataattatctaggttcaagaaccattaactctccctgatttctcaagggcaaagatgtcaatgccacgagaagatgtttgtgttcattggtgtttccaaatatattactttttctttggctttgttggctatagataaaccagccaatgaattttgggccaagaagtccaaaacacccctatcccattaacagtaacagcagcatcaagaggcagcctggtatattgcacttaggaaatttattgattcatagctgtgttctcagtaaggcgtttaccactacttttcggaaaacaaattatcttttctgcatatatatttttctgcatttaaaagatacgagtctcttttttgagcagttctctttgtaagaattgatatttgatgactgtattctttcttaaaagaaattttatttcaaagaaaagaaatttgcttattttatagcagaataaaacttctatttttcagttacaatttgtatctattggaaacttttgtaaatctaagatacgacatggaatcttagtttttctgtatcttaaatattttactatttcatcatcctagaaattacttcatccatattcattatttccaactatgctaaaaaagactaaaataatatgaaaatataaaagaaggatagaatagcctagaaggacaataaaatagtgaagtaaatcatgatgttacatcatccttcagttttcttattgcattgcccagtgtcttgcatcatctttccagaagtatagaagaagtctgcagacactgtctttgtaatttttttgtttggttggttggtttctttcaggctttcattggacacttgagaattcagaatttttcacttttcacccagaatgatgacagatgaaaactgaccaaggaagatatttcacaagcattagacaaatcataatatgaatgtagatagatagtagcagcctagagcagagtttcccaacctcagcactattgacattttgcgccaaataattctttgttgtaggagccatcctgtgtgtgcaatccaggatgtttagcatccctgtcctctaccctactgcaggccagtagtgccatcctgctaagttatggctatcaaaaatgtctccagacattagccaaggttcctgggtggtcaaggggacacaaaatcaccctagttgagaaccactactctaaactctaatcatggtgaagtttattatataaatgaaatgtcagatgacaacacctaaactgtctctcagttatcttaagtcttctcaaactcaggataatgatgagtaaagaatatatttctaacaacaaaaaggaaatttgatagtatttctaaagacaaaaaggaaatttgtattcacattcagttagtcattccaccagaatgacttcatcacacaatattttgtgacaagaacctgaacagcctgttttacagtattcttttcatcttttattatatgcaccaaaatttttttttaaattttcttgaacctctaaatctacgttaaaaatttacctgatacactttctaaatggacaaatgccgaaggtagctgtgtatacaaatgtgactagaaggaaaaagatgatatagaaataaaataactccttgagttgatcattctgattggcatttatagagtagaaatgttttgtaattacagaggaaaaaagatggcctttccttcaacagttatgagccgtcagaattttcaaaaatactgcattttgacaatgtagtttctagtttgacaatgatatatttatcttcaaaaccaggaaaatgtagataagaatttggttttataatatttaaattcttattaaaatgtctaataaaattgttttccccatcactttattcttctgtaagttattttatatttaaaatgtaaacaaataaaaataagtaaataaacagtagcagcttcttttcctgataaatcgaggattgagtatgtattatctctttcctggactactggaataacctctccctccttccacagagaagccataataatctttatgaaatacaaatcaaatcatggtattcattctttaaatagctatcaataaaaataaaatcccaactttataccctgttcgcaaattttacgtggtctgaattcagcttacatttcttctttcccttgtctattgcccatcaggctcactggctttattccttcacaccaaactagttatttccggggtgggaggaaggcttgcagtgttttctccatctgcaatagtctttcccaaatcttagtgtggataaaggttccttcttgttacttgaatcacaaatactatgttctcagtcattctctgttacatcatccagagtacattatatcaattttccaatatttttatttatttgatttcccactataacagaggctctgttagtgcagggtcttttactattttgtaatcccaacagcaagaacaaaacaaggtacatagtacatatttaataaatacttgttgaacaaatatgtgccggtaatatttcttcatgctgctgaataagttaacagcatataaacacatacaaaccaagtggcatggatgtctgctttcatttttagccttttaaaaatatatgtaacccatcctaaggggtttatatttgttttgcataatacattaatatgtactcattattcattacacagttaatatatctatatttgcagggaatatacattgcttggaattatacaaaaaaatattatttttcgttttctaatattcaggatacagtgttttaatgggggtgtttcttcattctttttttcttactggtttttactttttaaatttgaaagctttgcagggatcataaggatctgttcaggcaaagaacatgaaagggtttacatttttatcattttagtgtttcttattctctatatcaaaaacattcacagataagttaacaagatcctcatcaggaggaaaagtaaattgttcactaccatcctctagtatcctaatctggtcttgttgttggctaacttcagcagttactattctgtgactggtgtaatattaaccaaataaattactggatttgttctacaaatattatgtcttagattggttctttcctgtctctgaaaataaagtcttgcaatgaaaataaattattttacaacagttaattagcaatgtaaaatttattgaaaatgtatttgctttttctgtaaatcatctgtgaatccagaggggaaaaatatgacaaagaaagctatataagatattattttattttacagagtaacagactagctagagacaatgaattaagggaaaatgacaaagaacagctcaaagcaatttctacacgagatcctctctctgaaatcactgagcaggagaaagattttctatggagtcacaggtaagtgctaaaatggagattctctgtttctttttctttattacagaaaaaataactgaatttggctgatctcagcatgtttttaccatacctattggaataaataaagcagaatttacatgatttttaaactataaacattgcctttttaaaaacaatggttgtaaattgatatttgtggaaaatcatactacattggtagttggcacattaaatgctttttcttactctgaattcctgatatgactttctttaggattgtttaaaatattctagtagttttaggtcaatttagatgtgatttagttggtctagatattataatttttaggggttccctttcatttttcttttttcttacgtttcttcaaatagtataatgccttattttcatttatgaagaaattaccctgctgttggtgatacgggtatatttaaataaaccagttgcagtgcatttctgcagaaagtccattaagacataaattttgtccagtaactacagtagaagtggtgactctatgattcattcatgttgcataagtaggtgaaaaatatgagctatatgaagagtggtataacatatattcataatttttcttaactgttaactaaatgtaagtacttataatccatttgcattttccttttgtgttctttgccattataactgtgcctaagtatatatgtaaatatatttccaactatagtgttaaacactgatgtcttttgaattttaaaaaagctagtaatgtaagaagtttgggacttcttaagaagattcatatggagaagttagacatgtcaaccttttgaacagcatgcaagaatgtttatgtttattttgtttctcccacacagacactattgtgtaactatccccgaaattctacccaaattgcttctgtctgttaaatggaattctagagatgaagtagcccaggtaaatgtatgtttgagattactagataactgttgtacaaattggtatgtcacttaaattgttttctctcagaaagtccacataaataaatgaaatagactaatagtaatatagtgtagaaaaaaacacccttaacattatttccatagataaaactaattagaactgtaaattctaaggagattatttatctaaactaattttaaaatcagaagttaaggcagtgttttagatggctcattcacaactatctttcccctttaaatatgatttattgtctttctcatacacagatgtattgcttggtaaaagattggcctccaatcaaacctgaacaggctatggaacttctggactgtaattacccagatcctatggttcgaggttttgctgttcggtgcttggaaaaatatttaacagatgacaaactttctcagtatttaattcagctagtacaggtaaaataatgtaaaatagtaaataatgtttaattacaataataatttattctagatccatacaacttccttttaaaaaacctactgcactaactagttttatgcttaaaaaaaaaaattattaccagtaatatccactttctttctgaaaaaattttctttagatcggccatgcagaaactgaccctgatttgtttttttggaatcacctaggtcctaaaatatgaacaatatttggataacttgcttgtgagatttttactgaagaaagcattgactaatcaaaggattgggcactttttcttttggcatttaaagtaagtctaattattttcccattaaattcttaaggtacatattacttgctttcttaatagatttataaatatgtattacttatatacttttgtttatgtttggctggaagagttttccatactaaaagtattttgtaccagtgatgagcttctcaacttttgctctttgaaatttaaaaagcagtaaattcaaaactaaattttagtcatgaatgagagcttaaatatttttaaagatttttgttctacttaagtcaaattttctaggtccagatgaatattgctgtaggtttcactgtgtgtatggattacaatatccccaaacaaagaaaaaaatgttttaccttgaaattcagaacaatgtcaaactcccgtggttcttactgaaaaacaagctaattaagaataaaaaatgttttgtagaatgtgatatatgtagtactcaaaagttacaggtcataaaccatataacttttcataaatttagaagcagatttatatctaatatgatattttaagtgttaaaatttaataatggaacccagaagttaagttgaaaacaagaagcataggcgtgtgtcagaagagtcaaacagcattcactgagcgctttgttccctccctcttcatttgattatttttgtgctcaatttccttttttcatgtttttatatcttgtactgagattagtcaatgaaaactagttgaaataaacctaaaaactagatgtttatttaatcacatattcaggaactacctgaaactcatggtggttttgtttctaaattacaggttttgaataattttattattagtatgattgtaacatttattggatttcaaaaatgagtgtttaaattgtttagcaaagattatttgtatactgatttaagactatatatatatatttttaattttgcacgattcttttagatctgagatgcacaataaaacagttagccagaggtttggcctgcttttggagtcctattgtcgtgcatgtgggatgtatttgaagcacctgaataggcaagtcgaggcaatggaaaagctcattaacttaactgacattctcaaacaggagaagaaggatgaaacacaaaaggtgtgtgactctagtttgtgtttgagactcttttcactgcagtggggcagagttgtttagaagcccagtgtatatacagatcatggtccttggaatcaagcagattaggatttggaaccaagttccactgcctctcagctgtgtagtgttagacacgtcatgcaggctctcaggactcattttctttgtctgtaaaatggaaataatacctgcttcataaggccattgtgagaattaaattacacgagatatgcaaagaacctatcacaatccttggaacatagaaggtggccaataaatgttagatccctttactttcccttcctttctcttattcaggtccctaagtatttacagtgattatttccttattctgtcatttattgtctctcagtaatgaccctgaaaatgagtggaaagaagttagtttttacatttccaagtttaaaatggatttcgagtcactcagtaaatatatcacactctagtcatctgctgtctagcttagtataactaagagtaggaaatacaatgtaaactttttttttttttttttttttttttttttgagacagggtctggctcttttgcctggcctggaatgcagtggtgcaatttcggctcactgcagccttgacctcctgggttcaagccatcctcccacctcagcctcctgagtagctaggactataggagcatgccaccacttccagctaatttttgtatttttagtagagacagtgtttcgctttgttgcccaggctggtctccaattcctgggctcaagcagtctgcccacctcggcctcccaaagtgctggaattacaggcatgagccactgtgcctagcccaatgtaaacttttttatgaccttttcctacaccctattctttattcaatccaatccaccacatcctaaattcaccacctcttacaattaaaaggaagctcattcctcacctctagtaaaaggaaaaaaaaaagaaaaatcgtatattagtgagtccccaaaggacagagaatgtaagttagcagtactgaatgaatttttccagcatcttcattactagtacttgaaggaaaaaaaaaaattaaaacaaagttattgtattaatgtaaatgtcatacagtgtgagttgattttattgaagtgtgatatataaattcaaatataggatggtagaaaataattgtactctttctgatactggaatttatgagcaagaagggtctagattgccctggaagaacaaaatttattgtatttatatattgtaactgagctgagaaaaaagaaactaaattttagatacatttgtaaatacagatgttcctcaacttataacagggttatatcctgataaacacatcataagttgaaaatatcgtacaggcaaaaatgggcattttgtagatgtgatgggatgcgaaaacacaaaacacagtatccaaaaaatgctggccacacagtacgctgtagactattggtcgtttaccctcatgactgcatggctgactgggagctgcggctcgctgctgctacccagcatctcaagaaaatactgtgttgcatatcactagcccaggaagagataaaagtttaaaatttgatgtatagtttctactgaatgcatgtaggaagagataaaagttcaaaatttgacatataatttctgaatgcatgttgctttcacaccatcacaaagttgaaaaatagtaagtcaaaaccattgtaagttggagaccatctgtgtaggcatttcttctaagctggagtttttatatcacaaattggctatgatacagtggattaattaggaacatgaaattcattactcagatcattattggcttaaatgagattttgatagggaactaagattgcagtcatagaaacaagagcagttactttgagtggctttgtgttttgttgtaacaatgagtgataaaatataagaagcattgtgctaacattagagttgtcagtgatagaaatgcctttacatgaacaagagctatctagtcagtgatgattagattttctaactattcatttgtatataacaagaagaaaaaaacttatttaaattccagcatgaaaattaatttattataattcctagctcctaaccactttttcccccactcacattttatattttcaataggtgaaagtttcctaaaataaaatcccagctattgcgtgataactcagcagtctgggctctgatttgaattgattaaaaagggatattgttttgtagccacatactaaaacctgaatatagtatgagactccatgtaacaaatgtttttacaagtccttttcccaacaatttcttttagcctcactagaccactttcacccagcaaagagagtatgaatgctgtgttttcttaaaagcctaaaagtaaggttattaagctaatgtaacatggtctccttagtttttcctctagaacacagatcagcaagttttcttaaagggccagatagtaactgttttagactttgcaggcccatgtggtctctgtcttaactattcaactctgctattctcgtaaaagcagccaatatgtaatgaatgggtgtagctatattctaatacaactcaatttacaaaaactggcagctgacctgccggttgactagccctgctctacagccaaaagcattgccactatcaccaagcagatttcatcttcttacaggaaggggtacacagtgctgccagtcttgcttctgtctctgagtgttgctgctctgtgttgtagaaaccctcttaattagaaagcagcagtctaggatgtagattcagagactgtacagtactgaggttctcatgtgagaaagagattagcagttagttttatcttttattaagtcagtttcttactgtgactatcctttttttttaatcaggtacagatgaagtttttagttgagcaaatgaggcgaccagatttcatggatgctctacagggctttctgtctcctctaaaccctgctcatcaactaggaaacctcaggtactttcttgggggtttcattgatatatttaaataaataccttttctggataaaatcttgagaaaagtaaaaatgtctgttataattagaatgttcaataatttatgcttctctctctcattctcctaccctcaaaataagagtagtatatcttaagttcagtactgcctttattcagaatgagtttttactacttaaataatacagtttaaaaccttctatggccagaatttctgttaccataggataagaaatggaaatgtaatatctgtaaaactaatgatatatctctatatatttgttggaaattcatatgcaattatataacttttaaaacttttagttttttttatactctttaggaatggattcctaaataaaaattgaggtgaaagttgtaaatctttgtaacacttcaaaaagctatattgtatttatattttaaaataaatttcagggtaaaataataataaagcaaaggtacctagtaaagtttttaactattttaaaggcttgaagagtgtcgaattatgtcctctgcaaaaaggccactgtggttgaattgggagaacccagacatcatgtcagagttactgtttcagaacaatgagatcatctttaaaaatggggatggtaaggaagagtattaatgagcttatgatgcatgaatttagctatctttttatacacaggatatttatgaaccatgaaaactactgaaagccatttaaggaatatacacatgtgataaaatatgtaatatttatcagatgtcttgacctttgaaatatgcatgtataatcaatgaaaagaaaagaagtactaggtttagatcagaagtcctgaaatcagttttttgttttttctttttcctgttccctgcctccaacccccctcccgtggacctgtgtagagaagtattttttgttgttgttttggttttttttttaatcagtttctaattatcatttgcttagctgtgtgagtatccattcattccataaatattctatgtgctaggccctggggttttagcagtgaagaaaacagaatccctgccctcttggagcttacagcctaacagagaaaagagggacattaaatgaataattacaaaaataaaattgcaagcatgttgcaaaggaagagtgtagtctgctcaggaaccatataaaagaaggtacttatatggaggaaaaggctatggggggagtgaaggaggatcatggatgggttttcaataggaaatgacatttcagcagtgaattctgttgatcccaatattttaatataaacatgtgttcaaatttaaggattcagaaaaataattactataaatgctttaattaaaaatttttgtgagatgcatgctcacttatcctacattgttattcaaatatgtttatgcaaagactttaaaagtggatttaagaatcagaatattaagagccatttgtagtggtgcatgcttgtgatcccagttgctctggaggattacttgagcccaggagttttagaccagcctgggcaacttagcaagaccccatctcaaaaaattataaatacattaataaagctatgtaaaggctttgagtggacatatacactcatttataaattttttgaaaagaaatcagaatattgctttcctgaagtttcttttgaagagtaaatatagctgtatttgtttttcatttgaaaaccatgtgatggcgtgatccccaaatttgcatctgtggcattaaatggtgatacatattatttgaatttcagatttacggcaagatatgctaacacttcaaattattcgtattatggaaaatatctggcaaaatcaaggtcttgatcttcggtaggtaaccagtaaggcaacctgtatgttgaaagttatcctgaaaaagtgaactattaataattatagaagcatatagaggcatatgtctaaaaagaaatgtatgcagtaattatcagtagttgattacactatagtactttgacatatcctcctcttacttagaatagctaaattatattcagctaagtaaaaagagctaataagctaataaacactctgctactgcctctggagtgtcaccattaggaacaagacaaaggagacacaaaagacccctgttttcattcagaaattaagagtttaaaagaaagtttcataatgggaattttctaattctaaaactgtataaatcctcaaatattggaatcagttttgcagcaaaattatgcaaccagctatcttagagtttttaccagtctgttggtttctacccaactttccacaatatcagtattatcaccaagttttctgtcatgcttcatcttctcctagtcattgttgcttctaaaatgttcttccttacctgacttgtctttttgcaaatccatagtcattcaggcaaagcccactctgtccaaagtattttggcctataagtggacatagagtcaagtctagctgtaacaacagggtacatttagtaggaattgcaggtaacaccagacaagggcttatttgtatgcctcttataaattttaagagtagctagtatacaaaaatgtgtcagtgacaaatagttgtacccttttattgcaaagaagaaagtggacatttcaggtgacctgctaaggcacaaaattgtcatcaacttcaaatttgtaactcctgatacgtagcttaacccctgctgctttccaaaaatggttaatgttatatgaatcaaatttcattttttggtataatttcctgtgtcatgagagatccacatgccactactaccatgtcccccaaaggaatagactgtaaaaaagattatagagctaagaaagagtaaaggagataaccgtgtaacgctggttaattacattgtttaattataaagtaatgactttgcaaaaaagcatctgaagttaagatgattagaaatattaatggttttttagtcttgtattttctgaatattttttaacttaagtaaatataagtaaaaattttatttcaagtttaactttagattaaaattatttaagtaaaatcataacagcatttttatttatacttggcaaaactataattcagaatcgggtttattttaaccctgcttcttttttttaataacaagacttagaagacaattaaattctgtaaactgtgagcatgttcaacagtgataaaaacataaaataagaaagaatgagcaggagtacaaccttaagatatttccctatttacgagggcaggtagaagaaaaggttgtgtaagagaagtcatcaaaggaagttggagtactagaggaagtgattgacaatttgtagggccagggcttcaagggggttaaaagaataaggcctgtgaaaaagcacttggatttagtgactcatcattagttttaagagtttactcactagagtggtaggtgtagaagctaggctacaaggaattaagtaagaaaatgaaaataagtattgaccgtattaggaatttaagaaataaaggaaaggtggaaaatatggagagtagcttacgagaagaacagagcaaggaaaaccaaactttggatacttacgtgtcaatctgaatgttaggtgctttttttcattgtctctttaaatcctcacatctgcacccttgactaagtgtcctatttaattttctatgtgaataaatgagaggtaccctaaaaagcatttgtgccacatcccaagtattatggttgtatcaaagggaaacatttgtgggattatttgagttgtgagctgaactagctgctgtttttatggaacacgatttttacttaaataactagtaggcaagctatggttattcagacatggatatttggcagacattttcttgaaaatgaacaatatgtgcctgtctctttgaagaaaacaactgtgacagtatttgttgccaatgataaaattcaaacttgcaacaaaaatttcattagaaattcaaaattagaattttgaaaattctaattttcattatctgccgctgaacctaatagagtccctaaacttaacatctttctgatgagattcttggtaatattaacaattgtggttttttagtatggtataatgaaaagtatcaacatttggaacatctacaaatgaccatgcaggatactataaaatcatgtatgggtaagagatccattaaaaggcaagaaagacaaattgattttaatgtaactgagtatgaaaagtttattgatacagttttagattacattttgcaacttaagaaacttatcttgtcaggttttagaatagtaccaaagaaaaatatcacaattatctgaaaagtctattataatactccttctttttctacctacctatctttgtgaggccagattttctttttatactttaattaaaacattgcattagattgcatacaaaagcatatatgagaatccagctgggtactatgaagccagacatttaaaaatttttttaaatgtgaaagaatttcactcttctcactaaattttttaaatgcattttttgtttcatgaaatatgttacttatattaacatgtcatagtttgttgcttttaaatgaattagtaaatatttaaaatttttattttccctatttcaatttctaatatcattagatgtaattcacataactaaaagctctttggagtcctcagtaatttctacaagtgtaaaggagtcctgagaccaaaattgagaacttctgccctacagttttatttcgatgtcaattaaacatctttaaattaacacatatttttaaaatcataaatgtttgtaaaattttaattgccacaataattttcttaattcttttctgataaaaatacagtgctttctcagttgaaggttataataaatgatgacaggtaattttgaggattattccaggagtatgtttatcacaccataaaaaagaaaattaaaattggggaaaggcagtaaaggtcatgcatgacaaatttactaataaaatactcatgttttagcctgttaaaacatttgctattttaaaattccatcatttaattgtaaacgtgttactcctctttcagaatgttaccttatggttgtctgtcaatcggtgactgtgtgggacttattgaggtggtgcgaaattctcacactattatgcaaattcagtgcaaaggcggcttgaaaggtgcactgcagttcaacagccacacactacatcagtggctcaaagacaagaacaaaggagaaatgtgagttgtattattctttcttcctatgttaatctaagtttttgttagatgagtctgtcggtgtttgtgtattcctctgagttagaacagagaaaacaattgtactttctatggaaaaaaatatgctcaacctttgaaatatttgatgttaatggatttaaatgattataattacttttaatttggtaaaatcttaaacattcatcttatgtattatctaaaatgtattgttattgcttattctttttaaaacaaatgaatattgcacattcaaaattttatttctaattcattgttaaaatgattagaaaaaaataattttaatgacatgctaagtattttttcacatgaagaattatgctttggtcagggaacatctggaaatttccttagaaacccatgaaaacttcacaatctcaaaatctttggacataatttccttattcgttgtcagtgattgttttcattgtttaaatggaaacttgcaccctgttttcttttctcaagttggcctgaatcactatatttccatactactcatgaggtgtttattctttgtagatatgatgcagccattgacctgtttacacgttcatgtgctggatactgtgtagctaccttcattttgggaattggagatcgtcacaatagtaacatcatggtgaaagacgatggacaagtaatggttttctctgtttaaaatgttttggtgttcttaatttattcaagacattttgtatctgcatatatcaaactataacataatttcttatttttgaaagctgtttcatatagattttggacactttttggatcacaagaagaaaaaatttggttataaacgagaacgtgtgccatttgttttgacacaggatttcttaatagtgattagtaaaggagcccaagaatgcacaaagacaagagaatttgagaggtgagctcgagcaattaaaaacacaaaataaagagttctggctgctctattagaaacaatcaatatttttcaagcaatttcaaaataataaatgttggctgggtgtggtggttcatgcctgtaatcccaactctttgggaggccgaggctggaggatcacttgagctcaggaattgaaaccagcctgggtaacatagagagaattcatgtctacaaaaaaatttaaaaagtagccaggcgtggtggcatgcactgtagtcccagctactcaggaggctgagatgggaggatcacttgagcccaggaggttgaggttgcagtgagccattatcacgccactgtactccaacctgggcaacagagtgagaccctgtatcaaaataaataaaatgaaataatagaagttcttttaccacttcagcaaaaactattttttgtttgttttttattctatcttatttcaatagcttttggggtacacgtggtttttgattacatggatgaattaaatagtggtaaagtctgagattttagcgcacctgtaacccaagtagtgtgctttgcccccagtatatacttttttatccctcactgttcttcccaccgtctcccatctgagtctctgtagtccattatatcactctgtatgcctttgtgtacctatagcttagctcccacttacaagtgagaacatactgtattttcaggttttccattcctggcctccagctccatcccagttgctgcaaaagacattatttcattcttttctgtggtggagtagtattctgtggtgtatatgtacactttctttatccactcattggtcagtgggtacttaacgttggtttcatatctttgcaattgtgaattgtgctgcagtaaacatacgatatgcatgcagatgcctttttgatataatgacttcttttcctctgggtagagggattcctggaccaaatggtagatctacttttagttctttaagaaatctccatactgttttccatagaggctgtactaatttacattcccaccaggagtgtataagcattctcatttcaccacatccatgtcaacatctgttgttttttgactttttaattatggccattcttgcaggagtaaggtggtatctcattgtggttttaatttgcatttccctgatgattaatgatgttgagcattttttcatgtttgttgaccatttatatatcttcttttgagaattgtctattcatgtccttagcccacttttttttttttttttttttttttttttgagatggagtctcactcttgttgcccaggctggagtacaatggcatgatctcagctcaccgcaacctccacctcctgggttcaaagcaattctcctgcctcagcctcccaagtagctgggactacaggcacatgccaccacacacggctaatttttgaatttttagtagagacggggtttcaccatgttggtcaggctggtcttgaattcctgacctcaggtgatccacccgcctcagcctccctaagtgctgggattacaggcttgagccactgcatcctgctttaacccactttttgatgggattctttgtttcttgctgatttgagttccttatagattctggatattagtcctttgtcagatgcatagtttgggaatattttctcccattctacgggttgtctgttaactttgatgattctttcttttgttatgcagaggctttttagtttaattaggtcccatttattttgtttgtttttgtcgcatttgcttttggggtcttagtcatttattatttgcctaggccagtgtttaaaagagtttttcctaagttatcttctagaatttttatggtttcaggtcttagatttaagtcttcgatccatcttgagttgatttttgtataaggtgagagacagggatccagtttcattcttttacatgtggctagctagttttcccaccaccatttattaaacagaatgtcctttctccaatttatgtttttatatgtctaagatcagttcgttgtaagtttttggctttatttctaggttctccattctgttccattggtctgtgtgtctgcttttgtgtgtatctgctgttttggtaattatagtttcctggtataatttgaagtccagtaatgtgatgcctccagatttgttctttttgcttagtcttgctttggctattcagactctttttggtcggttttatatgaattttaggatttttttttctaattctgtgaaaaatgatggtgacagtgttttgtagttttccttgaaaagatctttcacccccttggttaagtacattcctaggtatttttttgaagctaaaagggcttgtgttcttaatttgattctcagcttggttgttgttggtgtatagcaatgctgctgatttgtgtacactgattttgtaacctgagactttactgaattcatttatcaaatctaggagtctttttttttatcttttttaagacagagtcttgctctgtcccccaggctagagcgtagtggtgcgatctcagctcactgcaacctccacctcctgggtttaagcaattctcctgcctcagctttccaagtagctgggattacaggcatgcaccaccatgctcagctaatttttatattttttagtagagatggggtttcactttgttggccaggctggtcttgaactcccaatctcgggtgatcctcctgcctcggcttcccaaagtgctgggattatagaaatgagccaccacacctggctgaaatctaggaattttttggagaactcttgggttttctaggtatatgattatgtcattggtaaacagctatagtttgacttcctcttttccaatttggatgccaatgtgggcttccttgtcttgttctagttctcagggggagtgctttcagcttttccccattcagtatgatattggccgtgggtttgtcacatgacttttattattttgaggtaagtcccttctatgcctagtttgttgagtgaaaaactattttaattttttttttttgtatttatttgtgtgatgctgtgaaggaaaatggaaagggaatacaatttaatttgttgagctaattaaggcctaaaaagaaagtaatccttaaacttcataacacattaaaggtttttatttactgagctttaaatagttggactccacctctatattgacaaataatgtatagtgcttaatacgacattttttggtcatacactttgaggaaagtcagtcaaccataatcaccttgtttattcataacttttttaccaccttatggtatctcattagactatatcagactttaaagtactttttacaatcttctataaaattctgctttgtctacaacacagtcctgactctagcttaagcacaaaaggatttagtatgaaccaattcatacatttaatacttattaaactcctgacatgccaggcattgttgtaggtgctggtaataaagcagtttttaaaaagtccttattctcttgaagtttacattctagtggggtaaagggaatcaaaagatgttggtaagagaagtgagagaggaatgctatttttttatagctttgtctacgaaagcctctctaattttgtgacatttgagcaaagacctgaaggtattaacatcatttgctccaaactgaccaaactgttcttattacttataggtttcaggagatgtgttacaaggcttatctagctattcgacagcatgccaatctcttcataaatcttttctcaatgatgcttggctctggaatgccagaactacaatcttttgatgacattgcatacattcgaaagaccctagccttagataaaactgagcaagaggctttggagtatttcatgaaacaaatgaatgatgcacatcatggtggctggacaacaaaaatggattggatcttccacacaattaaacagcatgcattgaactgaaaagataactgagaaaatgaaagctcactctggattccacactgcactgttaataactctcagcaggcaaagaccgattgcataggaattgcacaatccatgaacagcattagaatttacagcaagaacagaaataaaatactatataatttaaataatgtaaacgcaaacagggtttgatagcacttaaactagttcatttcaaaattaagctttagaataatgcgcaatttcatgttatgccttaagtccaaaaaggtaaactttgaagattgtttgtatctttttttaaaaaacaaaacaaaacaaaaatccccaaaatatatagaaatgatggagaaggaaaaagtgatggttttttttgtcttgcaaatgttctatgttttgaaatgtggacacaacaaaggctgttattgcattaggtgtaagtaaactggagtttatgttaaattacattgattggaaaagaatgaaaatttcttatttttccattgctgttcaatttatagtttgaagtgggtttttgactgcttgtttaatgaagaaaaatgcttggggtggaagggactcttgagatttcaccagagactttttctttttaataaatcaaaccttttgatgatttgaggttttatctgcagttttggaagcagtcacaaatgagacctgttataaggtggtatttttttttttcttctggacagtatttaaaggatcttattcttatttcccagggaaattctgggctcccacaaagtaaaaaaaaaaaaaaatcatagaaaaagaatgagcaggaatagttcttattccagaattgtacagtattcaccttaagttgattttttttctccttctgcaattgaactgaatacatttttcatgcatgttttccagaaaatagaagtattaatgttattaaaaagattattttttttattaaaggctatttatattatagaaactatcattaatatatattctttatttacatgatctgtcccatagtcatgcattgttttgcaccccaaattttttattgttcatagcagcatggtcagctttcttcttgatctatagatgaggctcaggcactatcccatttataccaataaccagtgtataactacttaaggaaaacataaaaacttcatcttctttccttttatttcttatgtgaatctcccgtcttccattctcttttataattgagaatgtctcaatcatatgaaattagttaccagaattaacacaatttagactatcttcctgattccttaaacccctttactgaagtatactcatgaataatactttaaaatatgggggaatagaaaccatgaactttttacctttttaaactatttatccatatctccaaagtagaacattaaaccattttaagatatgtctcattcccaagtagtcagagctcactctccaactttattaaatactatttgagcacaggacacattcttaaacattttgaaaaacattaacccaagatgtagaggctactgctagtcgtcattctagaatctgatattttactctgtatttgaaatgaatgattaatgtcctaggaaattagctttagcagatgtccaggtgccacatcaaaaaagtgcaataattattgacagttttttagattaggcatattattggaaaacaactttataaagagtgaacattgtatactctagtaaaacagcatcactttaaaaatattcatttatgaaatctgttacctatagttgaagtcttgagtagtgaacaagggactctaataccaatactcttaatatctggctattttagatcccttaaagggcataattattggaaatttaggtatttcactaaagcatgtatataatattgccaacaagaaaagtaaatttgaagattaagggaacttacttctgcaaactgtcttgcgatagttaagcagaatttaaactctgttttaagcaggaaaccagaaagattattttgcagttgtagaagatttcataacttattaaaacttattaacattttgtgttgtttagatataggcagttgatacatactaacatcccagccttttcaatatcagggttaaattataggaaaactcagtaaaatggtacaaatctgaaagtttgatggtagaaactgaagatttaacagagaactgtgttttacccgagtgccaaaaatgctgtgagcctccttgcacaaaatttataccacttttgcatttttatctatcagtccagatagttgtctcccctccttctcccaggacctctccaccattaaaatgcacaaaccacatggccgatttcaccatttacatttat

SEQ ID NO: 12 - DNA, 33396 bp, Homo sapiens
cgggcggggcgggcagagccgggagcccacccccagcgctcacctccgccgccccgagctcctctccggtcgccgctcgcgcgcgcgccctccccagggcggccctggcgcgcgtggagctgcgggcagcgcgccccggcggggagggcgcggggacggcgagggcgccgggcggggcgcgctccaacttggctggcccagcgccgcccgccgctcgccccgccccagagagagcgaaactcggggcgggggccggagggggcgggccgagcgacttcccggagcggcggaaccgctgaggtcaccctcgcccccccttccctgtccgccccacccggccgggccccgcgcgcagccagcaggggcgctggggtgtgaggggggtttagggcactgcgcgggggcagggggtctctcgcgatacttggcgctttggcctgggtcccccacctcccaacctttcccggcatggggtccttactgcgccagtctgcaaaaggaacccttccgagggtccgcttgtatcccagcccgcgttgggcagtcacccgagggtaaacccgggaccctggcctcccgccacctcgcagggcgccggccttgggatggcgtaggggttgcgtggattgggggtcgttaattttgcaaagatgacacctccttcttccttgaaagcctctttttgaatttaattcagctgggaccctcttccaaacattcaagcaggatgtcgatcctagagtccagaacggggacttccaccctctccacagaccccagcccagcgtctatgggggacccagcgcagagaggcacggaaaacgggcacctcccagcagacatggtggcacggggggacaaacgccataacagagacagtcggaagccctccagagcccagggtaggcaggaacgtgttcaaagaggaggagactccggacaggggctggcatgggagtgggggtagggaggagtgggggaaagtgttctagggcaaagaagcattgagaagatgtgagggggacgcctggttgtgggagagggaatcgccagagaagtagagagtgtgtttggcggggttagggtccctcccaggcagatggagccttttcccaggggcacaacctgggctgcccttgactgagcaggtttgaaggtactctagaccaccggatcatcttcacaacagtcccattaactagacgctcttattttcatacctttttggtgagagagtttaggtaaggtacccagagccatgtatcaggcagtaagtcactgaaccacgatttgaattcagccggtctcattcttctctgccttggagtccgggaaatggagaatgtagtggaggagtcaccccagtcaaggccaagcagtggggtgggggtggtgctggaagaggggctggatcacaaaggacaggacccggccaggtatgtgtgtgcaggaggagtggagcccacagtgggatctgaagtctgggaaggaaggcctcgttgttatactaaaatatcattccttgtttcaccggatggactgcatctttatttcctaaacctggcaactgcagcttggacccctggaatgacactgcacttacagccactgagactagcttagggacggcagaggcctggctcccactgccgccggccatccaccctaaggaccccttcctgaggaaggtcaactcccctctctccacgcatttaaataatcatctgccttgccccagccaccagcaggaatagctgaattcaagtaagttaagggtgaactcagatgtgactgctcccttcctgccctgcctggatcacagccctgccccggcccctcccaaccttgagcacgaccaaacctttaacctctttgaacctcagtaatcctcatctgcaaaatgggagtcagagtgcatcccggggtcttcgtgaacattaacgacagggcctggcactcagcaagctctcaggctctggaatgttcttccccaaagaacccttcactggtttctcttcatccctgtctctgctgaaagtccactccttagtcagggtggccattcctgcccaccttcgctaagttccctcgcagtcccttctgctgcataccctcctttccagcccattccgggtgtcctgtgattgtgggatattccagcaccttctgaaagggaaggggctgtttggtcacttggtgcttaaaagttggcaggacgcaagtgggtgttcagtaaatgtttgttgagtgaatgtgtgacggttgcttctgtgagtgattacgatggtcccaccatccctaatcctgactcctctgtggcaacattccagccctgcctgcccacacccccagcccctgaaccttcagagcaagtaggggtggcctgccacactggggctctgggcaacaggctgacagctccccactgcaggctgggagcaagcaccctagcctgccctgcctggctggcgggacagagagagcctggacacttaatgggggttgtggtacacagggagggtcaggatgctgccttcaggagcctagggtaggacctggaaggagggcccagctaggaatgggctagatgaaggggaggggacagtgggagagaagctccttatccctctcaggtcgggatgtctgggttcaagtccctcctgccccacaccgatcagggaaggggggctgttccaggcagagaccgcccagagttggtgctgggatcctggccagcctgtagtcctcagagcccctgtgactggctgggtctcaagtgcaggaatgacaggacaccgtcacacagccactgtcgcggcggtggtggttcaggcagaacctctgcagactcaggcggccatctggcctgggagctgccctgaggccccgccctgcagctgctgctgggacctacacagcccagggccctcgagtcctgcccccggccagcagctgcagaaggtgacagggcaggaggcccctgagctctgggaggaggcagccgtcgggcccttccctggtgcagcagtttttgtcggaaacatctggccacagagcacctcagcagtcccagcagcagccgtggggctgggttctctcctgaccattctgtctggggtgggagggggtgttcttgaagagaggactctgctttccctctgggacgctggaggagagggagatggccctgcccactgggagatccccgtcttgggggagggcagttcatgccaaggtgaagacaggactggagtgtggggcccacagtgggttgcaggcaccagcagatgccagggcaccaggctgggaagactgggaggcctccttggagacagccagtgcaaaataagcaagaggtccaaccacttcaagaacagcccgcaatgtcactgcctcttccatgagagggtgccctgctccctcccacatccccaagcctcggccacttgctggccttgtctcatggccctcttaccctctgcttcctccctgaattccttcctccatccttgcatggcagagcccaggatggtggacagataccaggaactcttcctcggggagcagtgggcaggtgggacagtcccagagggagccccaggtggctcatggtgcaggtttgctgggtgagagacagacaccagacgcccaggctctgggggcaacccttgtgtcaggtatcccacccaagcggcgcagaccactgggcctctgacttggccccctggaggtgggcccttcctggggttacaaagtctgagctgggagggagcagaggtgctcaggcgagaggcaggaatggggagtcccctgtctgaggagcgagtgccctcatgcagggggagagagtccttctcttggtcagccccaagaggcagggatgaagcccgagttttggctgcagactctgtcctgagcactgggccgaacctgctgcctctgtctgcatctcctcccaccccctcccctgctgccagctgaactcactctggctgaaaataaaaatgctcccccaaccccaatacttagcagcctcaggaaggtaactcacaagagtctgtctttcacaaaagggttattatcaactgtgggcctctggaatgggggaggggcagttgtcggaggaacttctggctaggaaggggtcggggtggcagtcacctgcactctcctgtgagtgtccaggagaaaggggtctgtgtcccacttactcattccatacccgggccatctgcctcccagtggacttcggactgggtgtccgggctgaccaagtccacccagcagggagggctctgggctgtcagctgtcacagcccaccctagacaccgctcctcctctgtctggtgtctgctcccagctcaaggtgggagccaggcaaggtgtgcccctcttctcccactccccagcaaagtcccccttttgtgagtgtagctgccagtacctaggtgaatggttgactcccctcggagccttcctgctccttggaggcccacctccaactgacctcctgtcctggcatcctcccgcacttcccccagaggcacaaggtcttgggattgggccttttaggtcactcttggccgccccctccacgccccccagagtggggcagcctgaaagtcaacctaagccacccccgccccgcgcgcggaggcagaagtccgggattcgtccctgacctgtctcgggccccttctgctagggtcacggcagagcgagcccagaagcgccgggtcactttacagacggggaaactgaggccagagagggcgtggccccgggacgggctggctggtggccgggctccgttagcggcccagcccaggccccaggtcgtcgggagctgcccctcagcccggccgcgcgctccccgagcgcgctccgccctcgccgccggccgcgcggtggggacgccccgggcggcggaggcggatgtgggcgggcggctccgggcgcggggcgggcgcggggcggggagagggcgggccggcggcggcggcaggaccgagcgcggcaggcggctggcccagcgcagccagcgcggcccgaaggacgggagcaggcggccgagcaccgagcgctgggcaccgggcaccgagcggcggcggcacgcgaggcccggccccgagcagcgcccccgcccgccgcggcctccagcccggccccgcccagcgccggcccgcggggatgcggagcggcgggcgccggaggccgcggcccggctaggcccgcgctcgcgcccggacgcggcggcccggtgagtccccgcccgccgtggccgcccgggcctggatttcctccccgcgggccgggccgctttgttcgcggccggtcgggccggggcgcgagccgcggcgccgccagaatggaggagcgggagcaggaagtggccgagcgggcctgggcggggagggcgcggggcgcgcgggcccggccaagggagggcggccccacgccgggcgccgggggtgcaggctgccggccccagcctccctcatgaccttggggaggccgctccgccgggcgaggccgggacccgagagaagggacgccgccgggcctggagctggggctcggggtgcccctgcgggccgtggggctccccgggcgctgggccagcgaggcagccggcggaggtgccccggggttggagaaagactcgccgcggccggccttcaagtttgtgggagggccccggaaggagacttcgtttcccacggacgaaaagttgtacgtggtggcggggtacccaggctagccacaaaggactgtgaccctcctgggccccggaactgcttcctgtcttgggtgggccctggaggtcctgcccgcccatcccagaggccaaggcttggagggcagctggggcttgccccttagattgagtatcctggggcgctagcgagcttggtcctgtcgggacggcctctgagtgctgccttggtcagcgggtgagcttgggcccctgctctgcagccagaggccgccccacattcactcctgggtctctcggccttgctccaggtggccacttcttgactgctttgagtccctcatccgagcgaagggcggacggagtccgttggtgggggtccggttgcctctcccgggagctgtgtagacttctcatacaccagggttctggaggcagatggaggagccctttcgaaaacagagtatttttttttaagttgtgacttaataatagtagcaagaatatgtgcttatggtaaaggcaggcggcaggtacggaggctgtgggaagtcggggtccctccgcccccacaggcagccctgtgctggcctggtgtatacgtttctgtgcagacgtacaccaccctgtgtgagcacagatgtatttttacacatggctctggacagctgtctgactctgtcagcagcaggccttggaggggctcaggcccgtgtgggggtggggggacatccagaggtctttgagtccagccctctgcctccaggccacgcccactcagtgtcgtcagagccccctgtgcctgaggcgtgcgcggctcggagccctgccctcggagtcctgcggtgccttcctcgagtctggcctgctttccatcctgctaagtacttggggcatttccctctttgggtaaggtgtggtcttccctgtcctggcattagacacaaggcagtgggccttcctgccattctaagtgtagcttaagacaatcagtgcaaagcaaccctttgtgggtgtccagcccttgcctcgggaggccagaaaggtggcctggggggagagcgtctaagctggctgtggaaagacccatgttgggatccattccacagaggtcgtcaggggtctctgcctggcctggaggtcccagagaggaccctcctcccctcaggaaggcccatctggaagggtagcagaggactgctcacaggaagagcatgcgaagtgctctttctggggatgcctgtagttggtgatgtgggaactgggttttgagggatgcctaggagttcatccatcagaggggaaatgaggaagccatgcaggatcaatggataaagtgtgctcaggtgagggttggctggtgggccgctgcagggcgggggcctgtccagtgctcccccacttacttgctgcctcccgactgctgtaattatgggtctgtaaccaccctggactgggtgctcctcactgacggacttgtctgaacctctctttgtctccagcgcccagcactgggcctggcaaaacctgagacgcccggtacatgttggccaaatgaatgaaccagattcagaccggcaggggcgctgtggtttaggaggggcctggggtttctcccaggaggtttttgggcttgcgctggagggctctggactcccgtttgcgccagtggcctgcatcctggtcctgtcttcctcatgtttgaatttctttgctttcctagtctggggagcagggaggagccctgtgccctgtcccaggatccatgggtaggaacaccatggacagggagagcaaacggggccatctgtcaccaggggcttagggaaggccgagccagcctgggtcaaagaagtcaaaggggctgcctggaggaggcagcctgtcagctggtgcatcaggttagggaggctgggaaggccttttggggatgggggtgatttgtccaacggctgggggaggtgggaatggggaggtgagcaaggcagcagctctcagggcctggctgttgcgggtggtggtggcaggggctggaggctctaagcctagaataaggagaggcccaggtccagggaactgtgttcaattacatggatttgacacttggcagccctgagtgttttggggagagggaaggcaggcgggcagatgggggtcagagagcttagagggatggcagcccacctgggaaggcaggtgcgggtggagcccccaggcacgtgcagtgggtctctggctcacccagggcgaggagctgcccttagccaggcgtggcctcacattcagcttcctttgcttctcccagaggctgtggccaggccagctgggctcggggagcgccagcctgagaggagcgcgtgagcgtcgcgggagcctcgggcaccatgagcgacgtggctattgtgaaggagggttggctgcacaaacgaggttagtacccgctgccagggctgggcctggggagggagagatgggggtagtagccccagggtctgtgagtgcctgtgccctgctgggtgggaggggctgcctcccctggggctcctgggctggcctagggtgagtgtcctgggccccatgtcttcccatggcatggagggcacggcctcggggaggccaggaggatgaggccctgtgcagggccggccatctgagtggcttcctggtgtgggccctggcaggcgggggagctgctgccagcctctctcctggctttcctggaaggcagcgaggacactgggctgttccagggtctggcacgctggcggcccagggcactgcccaagggctcctgagggcagggcagggctgcttggcttggcctggtgcctgcttcaggcctgagctttgggggtggggggaatccgtaccgcctgggcacagggcatgtttgccttggagctgcagagctggctgagccaagtcccccagcccctgtgtccccctcctcactcatttggccttgcagcctatggaggaggtgctgcccgaggggcctggccagcagctgggtgggatgggagctggggacatagccccaccccttccaaccctgtgcaggcccttctgcctcacccaccctccgttgctcagcggggtgcggtggagtctgggtggctgttttgctcctctgtcctgctagggtgggcttcctgttcaggtccaggtcctctggccaagtcactctcttctgccccaggcggaatcggtccgccctctcctgacttctttttccagacttgttccctcctaagttctagtgatctcatgccagcagccccccggctttacgtacaccctctagcagatgggtttcacatctggtagtggggagaccccaaacacagctggagcagacagggaggtgtggcaaaagtaggtgtcacaagatggggcattgtgggatgtataggagctcacagaagagcaggcctaaggagccacaggagcctgggggtggggtgaaggagtgaccgagatgagatgctgggggaatcaggggtgtggctggctgtgtgtttggagtgtttgagtgggcagctaccagtgatggcacgcggggcctcgagatgaagacaaggaggctgcagacgcctgtggtccttggggagcttctggctctgcttccccccaaggcctcctgccgcatcctgccttctccatcccctccccgtgtgctgtcccctgcttgttccccaggagtcctgccacctgtgcctggaggtcctctcctcacctcacagccctctttggtgtcagggaccccctgccccgaccctggccctgttcctctctgtgtcacatagtctgctgcctggggccgctgcttctttatccttggcaaatgtgtgtttgttgatagactgttggatctcgggctgacccaggagatgccagggccttgactgcccaggggcccaggccccagttactggggcacccatcagccagctgggcctaggcaggagtgctcccccgaggccaggtgattttcaggctgctgctgatgtcgtgccttcaggaggggcctggcagggacaggacatcaggcccagtggggcgtccagagagctgagtttgaaccctggttagccactctatcgccatgaccctttccaagagcaaggtcattgagctccttgggcctcagtttccccatctacaccagggagcggtggtgggagagagacctggtggagtggagaatggcttgtgggcggccctgagttggggtccggccagcctcagcagcctctgggctctgtcatctggcccctagcctcaggccaggagagagggtcctgtacctctacccccggctcttcctcctccatccccagtgtagtgggcgctgaggatgggaggtggcgtggatatgacatatgcccttggcaggcatagcccctcttgttgccgagagcaggtggcgtccacccgcatggccggccgctccttgcggaggaaagcctgtcctccggggcctgccgcatttcgccactcccgtccctggggtcccctgtgtgtcttgcagatgtgctgtgccaccctcctggcagggccagccccacccgcaacactggttagaggtactagctgccttcctgtccctagcagggctgtgctgggctgaccccacccgtgcttcacagtgctcaggcctggctgggggtggggagggagggagggacgcagtgactgtcctccaggcccggccgcagccccttgcagacgccctgttccactcccccccagccttgctcctgcaggtcagggtgcttgtctcagaggctggcccgcacctgtcccctttgccgggactccccacgaccgtggcctggggcactttggttctcagctgaatccccaggtgacagtcccaccacggctacttgggctggccacacgtggcgtgatggtgccttcaggggtgatgtggtcattgagatgagaggggacgggtatgccctgtgtgtgaggggtggggtggcagtgtctgcaggctccttctcctgctccttgggaggtgagcgtcatctctgggaagtggcccaggctgctttctgttgttctgatgtgtgtgctctgagtcagaggagcttcagggacagaagggaccgggcatggggacacaggagttccggctgcaggcctgtgtggacctgtggctggacccaggcgaggcctctgggtctgcacatctaacagggagatggttcctggagggtgagcttaggctgttaagggcaggagctagtgaggaggctggtggggcctatagggacagggggccagatgtcccctcctgtttcggctgtctcactggataagcagcccagctcagctctggccattggtggtctggtgggctcagtgttgctcagacgtttgcattttcaaagatttgttctgttgtgtgaagtctcctgatttttgaaggctgacaattttaaaaaatcaatcctgggccagggcgcaggtctggctgaactcggcctccagcctgcagttttccttcctttgatagctccgtgccaggctttggccagaggctgtgtggctcttttgaggtactttccccaaacagcttgctcaccttgcccgcttgccccagtactcctggggccgtgcaccagcctggctgcagcctagccccgtggagagctgccctggtgcagccccggcctggagcctgtttttgtccactcgcggctgtcttcctgagcgtccccccgtccacatggctggaccaagtgggtcaggagagggccgaggcgggtggccgggatgctgggacccaggatgaggtgctctgggctctcgagaaagggctgggcctaggccaggagtgggtgccggccgcaggggcaccacctggactccctcaggctggtcgctgtccgagaggggccggggaagagctgcaggcctggctgccttcggcaggctgtgacccagcaggccaaggccgagtgggcgggggtcgagggaagctgcctggggcgcgtgggttacgcggcagacgctcctgagtgtgtgggcagcgtgtccaagccacggatctgggtgcctggccgggcccgggtgacacgggaccgtgtgtccatgcggctcacggagggtccagtctccggacatctgagtccttgccatgccctaccccgggcccatgcctggcatagaccccccagtccaggcctcagggcagccccctcaccactcctgctgagttagccccaggcgtcccggtggggagtatggaggctggtctctgaccccaagcaggtgcctaggctgggcctccttcccaccccctctccccgccttgaagtcctggcactggctgtccctgcgggggggcactgaggcagggagagagtggttcctaccccagtgtgttacttcgctttggtgagcgacatagaacctcactgcatccccagctgctgctcagccatccagctggtcctggggtccctgcgtgaggtgggggacctcagagggtacaggccactccccttcatcctggaggaggccttggctttgctgagtaaacacccttgagaagaggtctgtgggggactctgagcccactccagagagcagctgctgcggagggccccgcagggctaattgtttgtgtgcagatggaggctgctcattaccccgccaggatgaggggcccagcaggaccctgagggagggagcccgcagactctcccctcagcctgtgggaacctttgggctccaggccagggctgcctgtgcctcctgggcctcagtttttcccaggctgtcaggtgagggtcaaccccaggctgcgtctaggtggcttcagtgtgagacccgcgggccctgggctggttagtgccaagctctgcctcccctgggggagcgttgagggtctggaacggcccccgtgggcaggccagggacagagatggggaggcaagtgtgctggctcctggtgggagggctgggcagagagtgggggtcccaacagccctgggcctacccgggagaggggcaggaggcacaggctggtggaggtcatggcatggctgtggtggaggccgccagcatcagggaggttcctgttttgggagcttgttgagttctcacttcaaaaacaacaaaagaggaagccgaggtggccttgagtgggggtgaggggtcactgctggccccagccctggaccttagcccccacgcctggtggctcctgagtggacagctcttcttcctgtgtgtttcttggtcactggccctctctgggcagggatgccctgtggggggctggactccgggaagggaaactgaggcccaggagagtggacagaatgtgggtggaatgattcctgtgctggggcctagacccccgtacaccttccactctcaggagaagcctggccgggttgcatcaggccgggttcctagaagtcctgcacgaggctgagcggagggaactggcgcggcccccgctggcgtcccctctgcctgggcacacccggcccggcttggggccagcaccctgccctcgggccctcccatagctgagactggtggctgtggctgtggctctgggcctgctccccaggccaccctgggtgggcagctgcctgctgcttctgtctgcttggaccattgtgcctagtcccatggcctcaggagaaggttcccccagcccagcgctggggccccgggcaggttggcagagggccctggggctccaggccaggatggggctgttaggcgaaccctaggccccttacttcctcatctccaacgggagttcccttccctgccatgccgcctttaggtgtgttttccttgaaccttaaggaatgataggtttggatttcagaaacgttggaattgagggggttgggagttcatgggtgactctgctgccaatgttgcgggctttggggtgggatccttggggcgctgtgtttgggctcacacaccccccaggcgggcccggtgactgcagaccccacccataccctgtaacctggcgggcccacctgcccaggccctcagctggttgtccagctcaggggtggagacaccccctgggcaggcctggcggggaggtggctgctccctgggcctggagaatgcagagaggcccttcggctggccagggtctggctggcctccccacagactggggccaggggcacagcagccaggtgtagggcgcggagacgctgagctgcacaagcttggtgaggtgtggggtgaggccacgggggtcagggccacaatgtcccagcaggccgggggatcagaaggagcaccctccacagctggccgtgctctgggggccctcctgccccaggcgtggctccaagttggggcagaaacaagatggtggggagatggaaggcccctctgtggcgggtgcttcccatggtcggtgtggaggacgaggtccctactgcctggttccagttcccccagcaatgcagggcacttttcctggggtgtcagggattgagaggccagcactggagcccttcaaccgccaggggccctgcccaagagagacgggaggagggaggggctgtgggcatggctgcgtgcccccggccgagccccggaaggagcctgtgcccgtgcccacttcctttcccgagggcagtgtctacacgtgggacactgtggccagccctccctcgcagcaggcactgtgtgctgtccccatgcccagcttcctcccacgcagggagcgagggtgagggccctgggaacccactgggtggagggcacttggcaaagcctgctgcaagttatggggagagtgggcgggggcagagtaggaggcgccagggaggggctgcagcctcatctgcaggggtcctgagtaggcagggtgggccatgacatgggtggggcttgtttggggcagaccaaaaatgaggtgggggacagggacagggacagggtcttgggatgtgctgggctctgcgatggatgggtgtgtggcaggtggggtgcctttgccggcccgaggggtgggtgacgggcagtgcctgtgggggaggacatccaggcccaggtggtctgtcaggggcagcaccagggaggcccagtaccccacccagtagagccccaggagaatggaaagctgagacccaggtgcttccctggagtggcaggaagagaggccccagcgcctccggctgtgcctcagcgaggtggacggggtcatggtggtgccgcctgcccgcaccatgttcctcccggggtgtgtgtgtcgggctgaggcctgctccatggtggagcggaaggaggtgaagaatttgcagtttgtgtcaagttacaacccctgccttggcggcaggacctggggtctctctggtcatgatctgcctctgagggtcacctcccctaagtctccgttttcccagggagctgatgggcatgatgagccctgcccgccctgcctgcctggtgggctttctgggagagcagaatgggtcctgggctgggcgaggcccaggcaggtggagcgccagtcctctgcctggcgggaacagacacctctccttgtgcagactggccacttgtgccccctcagaagggcttgtgccgggaggagcagtggatgtatttcacccacagacctgggggctccagccaagtggggtcagctggcctccgttgtgtggcccctcccctgcccccaccccagcctggttgtttacagggttcgagggctgctggtaaaggggtgtgtactcccgggcccgggcagaggcctggcaggccgggcccttcgcggtgagctgtgcggtctcctggcaggcggggagggtgggttgcgggcccctcatgggctgccgtcagcggagccgtctgctcaatgtgctggagaaggtaaatgggcccccggcagctgtcggggcgatggatgtgtcggcagtggggaccacggtggccgctgtcagctggcgcccgctcccagggcctgctgggctcactttgctgtggagaagcagctcggtgccgcctggcaccctgccagccgctgccaccaccgtctctgcgaagtgtttatccagcagtttcctgagaactggggactttgagagaatgttcagggagctgaggacgtggtggcctaggggggctgacacacctggtggctcaggaggtggtcagtcccgggacgtggagggccaccagctggccctcaggggcccacccagtcctgcccatggcgcgtgggtcctggcccccatgtctaagtgggtgccgctttggggggcctgacccacagtgtggcctgggacacgtgtgtgaggcgtggagagggtgggctgcctgtgactgtccatgtggtccccgtggtggtggtggggtgggttgggggaggccaggcaggagcagcttcctctcagaaccccggtatttggtccaggaggacttctgggaaggaaccctctttccctgggggtgctccagccccttcctggggatgagcggggttggggatggaaggtcactcctgccggagggccacccgggacatcgggatgccagagggaccagggctggggttgtgtggggcccaggaggtgcaggaggagtcgagggagcctggtctccgctgtccccccgggcagggacaggagtctggccaactgcctaccccccaccgagggcagccacctcccatgcctggagccctgggccatggatgtgctggctggtggggagggttacgggtttcatcaggcacaggagcccctgcccttgtggcttcacaggacctggtcttggataggtgcagtgtcacagggttaagggtcagggtggctcccacctggcccagggctggccttgagagaaccctgaagggtgggaccggagctgctgcctcccactgcccagggggccagcaggagggtgagactggaactgctgcttccccgctgccgagggaaccggccatggggcaggagggtgggaccggagctgctgcttcccccttgcccaggggaccagccatggggcaggaaggtgggaccagtccccagtgccgtcctccactcggccccccactcaggtcttctggcagtggtgtggggtcccccatccggacttgctgacatttctgaaggggcacctagccgtcttccctgccagaccctgaggcccaggccggggttgcactgggaccctgcctgctccaggtcccgggtctgaaacatgtgcagcgggtggggacttgggggttcttgagcccagcatgggggtgtgagagttagggactgaagaggccccctgggcctgaatgctgggcggtctccattgcccttgggagtggctgctggcaggagatgactctggctgtgtgtggggtggggcgtgccctggcctccccatttgttgcctgcctgtacccgtctggagtgagggcctggcgtggcaacctgggggtctgtgcccactctgtgtttggtgccctcagctgtgcaggcaggtgggtggggtggtgttgggggggtgactggggtggggtggggggatgccagctggctgatggtggggagatggaaggcccctctgtggcgggtgcttcccatggtcggtgtggaggacgaggtccctactgcctggttccagttcccccagcaatgcagggcacttttcctggggtgtcagggattgagaggccactcttcaagtggactgtactcctgcctcgagtggggggcatctgaggaccacaacagggtggggaactcaagttaagtcctggatgacaggttccagttcccctggggaccctcaggatagtgaaggagaagactccatgtgggttggtctctttgtgtgaggggtctgggagggcagccctggctgatgtgtctgtgctcccaccctcctgagggacccggagtaggcaggtgcaggcctgcaggcgtcctgtggccatgaggacagtgatgctgggccccgggtggctgtctcctccacctgccccgtcctgtgtcaccccagtcctggatcagggagtagtggggccctgccgcatctgctgtcggggctggccgcccacccagcatcctgtctgggctgaactcgttaagatgcgcgccacggccctggctgcagctggcacccccccaagtgatcctctagctgaccctctaggcgagagatgaagaaagagaactgtgtgtctctggaccccttaccccgggtccctggggatcctgctcctcccctggaggggtctgtgtgctgggctgggatgccgaagacaaaggccacgtgaggccccttcactcacacccgtctcctggggtaggccctgatcgccgtctgttccctgggtctgtggggccaggcgaggggaaggcactgaaagcccagggcccatttgggcctctgcccatcccaggactccccaggaatgctcggggctgggcacctgtcctctcccaccccccacgcctggtaagcgccgcccctgggcctgcccctcccgcacccccagatccagctcccagaaccccccagatgcagctcacagaacctggggctgggcttgtggcagcagggtcaggggcaaggctggggttgggcacgggagctgcgtgctcgggggcagcacggggccatgctgggcacggtgggggctggcagcagttggtggataagatgggtttcaggggtgccgggcctgggcgctcatcagtcttcccgctcagttagcgtcccggggagtggcagctggcaggatgggaggcactccatcaggggccgacagctccagactggcatgcaggcgggtgggcgtgagcagcggggtggcccaggatgggggacgggtggggtgggctgcatgcagccccctgagcctgctctgctctcactgagcgcccctgcctgcccgctgcacctgcaggggccctgtggctgggggaggggacgtgggaggcggggtggagctgcgtggcacgtatgtgcgtgtatgtgcacactcgggggtgtgcgagatcccaggtcagggacaggagccggcacccaagggtggcaggaccacgcctgctcggcagcccactggcaggggcatggagggcttgcggagtgggtggggtctcctgatgagtgctcttgggcacaggggcagattctgggccctggatcatcctccctgcaactgggtgttggccgtcctccagaggtgtcaggcagctatgagtgggctgtggctgtccccttcctggtccaccccagacctgacaacagcctcttcccacactcaggccctgctgggtggggtgtccccaccctccaccagcatccccccagcagccctggcttgggcagagagagggctggtcttgtggagaccacgccccgtggtgctggtgggaagggcattgggcagctggggccggggtaggagggagaggtcacctggccggggggtggccatgtgtcgagccttggtggctcacctctattaaagaactcccccgtggctccctggaaagtagaatgcctgctgggatgcccatgacccatgtcctctgagctcccctggggctgctgccctgctgggacttccctgtgcaccagggagggctggacgcgtctgggctcaccacgaagacccagcaggactgggtggtgggtggtctggtgatgggacaattgcttgtcccccacatccctccaggtgtccctgtctggggctcagtgtggcatgaaaacccaggccccagagctcagggaccccaaccccttaccctggccccctccctaacccctgcccagtgacccctcagatgaagcagttgcctaattagtaagttccctgttgttcaaactccttgcttggcaaaatgtaagtggaacatttttttccgagtgtcagtatgaaaaatggtacttgctgggctatcaccgaggacagctgctctgattctgaagtgtgaacgcctgcgtacggctgatgctgcaaaacttgagggagaatgttcccgtgtacctgaatgacccacgggtgggacgcggcagggccgacggcggcagggcctgagtgtgggtctcagcctggggacttggcaccctctgtgccctggggcccctgcccagctggctgggccacctccgtgtctggcttcatcggcagtccccaagacggtgctccaggcccctagacagggagtgcgatcccacggcagtgggcagtcctgtcccgcgagcccggccctcagtctgagtggtgctgacctctaactgtggacgccatgctccatcctcctggtgggtggcggcggggcggggggggcggccatgctgggcagcccacacaagccactgtcacctgctgtcgccacctggccgaccctggttgattggggaatgctgtcagccccgcagcccctgtggccatagctggggcccgagctgctgctggtgcctgctggggactggctggtttagggctgcctcctccaggaaggctgtgcagactggcccagacctcttcccagcctcctgtccatggtactccatcccctgcgtccagctggcccagcccgttttcagacacagctcggggtggctcttctgagaggggcgctgtggtggggcgagtctgccttcccgttgacccagggccgtttctgtcgctggccctaagaaacagctcccgtaccgcgcctgccccctgggactcaggcatcccaggcacatctgtcctggcacacccagttcctgcctggctgcctggcgagggtctgacgggtagagtgtgcgtggctctcaccacccgcacgtctgtaggggagtacatcaagacctggcggccacgctacttcctcctcaagaatgatggcaccttcattggctacaaggagcggccgcaggatgtggaccaacgtgaggctcccctcaacaacttctctgtggcgcgtaagtatccccttggcctctcgggattcagatttggggggttggctggagccctctttgcccaccaggccccacgatgggggtcccctggagcctccaagtcctgggctggcaagcaacaagcactggctgggtgggggtctctgggccgagtcccgaggctggctgggcaggggtctccgggctgagtcccaaagctgaggctggctgggtgggagtctccaggctgagtcctaaggccgaggctggctgggcaggggtctctggtccaagtcccgaggccgaggctggctgggcaggggtctccgagctgagtcctgaggctggctgggcgggggtctctggtccgagtcccgaggccgaggctggctgggccggggtctctgggctgagtcccgaggccgaggccggctgggcaggggtttctgggcagagtcctgaggccgaggctggctgggcgggggtctctgggccgagtcccaaggctggctgaccctggagcagttccctggtgcccatgctctggaagctggagggtccggacaagtggcagcagagcccagtctcagggtggacagctgccccgggggcccctcctccaagggggttagatagagcccatcagggcctctccttgggaaaccccttgggtgagggggcaagagtccattggggtctccagctctgaagcctccatcactcgaggaagagctcgtgcctctccctgcacaggctctcagcctccagaccccatggcagagcctttgcttcccctctgtgaggctgcctccttgcccatggaggggcaggtgcagaaaggccagcgggggaccttggccggagcatcccacagagatgagagacatgtgacgctccctgctccgccctgctgtggctttattccttacctgtagcgggggcaggtgcgaccctacccatgtcagacgggaaccctggcatggagacccaggcgccctgctaggtgggaggctggggacacgtgggcctcccagcatggtccccattttctgtgtccctgtcctggagcccagctctgcctgtggcagccagagacacgggttgggcatgcaggtgtgactgagtggctaattagggcccagttgtgggccatctgtatgtggcagggctgggtaactgcaggcctctgggatctgtctcaggcaggtctgtggtgcagagcgaggtgcccaccaggggctggccttaggttccatccccatgtgctgcatcggcccctcccctccaagtagctctgccctcctttctttgaggcctgcggtcctccgactcccttgaacttgctggacccccaggttctggtccagggtctgggaggaggttaaatgcagggaatccgtccccaggctgggcagggaaggactcccacccttggtgcctgcacgtataaggggaggtggggtcagtctggcaccctggctgctaggtggtctgtggccagcaaaccgtgccctgaagcttctagttgggaggggcagaggccttgccctgcaaagccccaggctgaggggctggggttggggtggggtttaccaggacctggttcctgtaagccgaggtctccttccctggctcttccagcctcaagccagcagtagcagggcagcctgatagagtagggcaccccaggggtgaggggagaccaggaaggcagacacccctgtctcctgacagcctcgtaaccatgcaggagactcactgtgacttgtcccacaccctccccagtgtctcctcggttggggctgggggcttcagggatcagggagaggctgggtgccagcagcgtgcctggtggcacctgggacagggccttgctgggggtgggtgctgtgggtgtgctcgccaggccgaggcattgggaaggaacgaggtggggagctctctggagaggcacacgggtgggtaccagctggaggtgttgcctgagcagctggcaaggctggggccttcagattaggccataggtgagtggcctggagcgcctggccaaggagcagagctgggcaggggccaggggctgtgctgggcctctgtccggggcttttctccggggaacggttccggggcagtgggctcgagactctggccctggggattgcatactggggcagatcggaccatccaggagcgagtgggaggtgggtgtgccggggcttcacaccggtttccacttctctggcctgtgttgacacaggccaccctccccaccaggctggtcccgagccccctcctggcccccaggcctggaagacagcttccttgctgcctggccaggcccccttgtggttgccagggcctcgatgctggaatgtcagaggccccccactggcccctcagcctcctcctgctttgcacaggctgggccagagcctgccacggagccccagcggctacacactccactcactcacacctctcagggccccgcacgttcccacagccctcagggtgtacgtgctgtaggtgtggctgtgccccaggtatgtgttccccaggtgtgcttgtgccccaggtgtggctgtgccccaggtgtgcgtgtgccccaggtgttcgtgtgccccagatgtggctaggtgtctagatgtgttctagatgtctgtgtattccagtgtggctgtgtcccaggtgtgcgtgtgccccaggtgtgtgcctcaggtgtgcgtgtgccccaggtgtggctttgcctccagtgtgtgtgtgccccagatgtatgtgtgccccaggtgtggctgtgccccaggtgtgcctgtgccccaggtgtggctgtgccccaggtgtgcctgtgccccaggtgtgggtgtgccccaggtgtgggtgtgctccaggtggctgtatttcccaggtactcagggtgaggttgggaaaaaagcaggacatttctacactaggtgggacaagcaggtttctggggtgcccccctcatgccagcccctctgtgggatctccatacccctcatcctctgaggcctggcgcacggcggggctcagagactgaaggaaggactgtaggagtggacgatggtgggggtttgacctgacgccgggccagatgccctgtgtggctgcctcagcctggagtagagtgtctgagctggaacaggggtcctagccccactctgtgaggcgagaaactgaggcttggagagaggaagagatggggcttcccaggacctggtgggtggtatgcaaggggagctggctggtgcagcgggggctgtggtgagggccctgggaggggcagggggatgcacgcagacagaggctctgcctgcccgcagagtgccagctgatgaagacggagcggccccggcccaacaccttcatcatccgctgcctgcagtggaccactgtcatcgaacgcaccttccatgtggagactcctgaggagcggtacgtgggctgtggcggccaggccaggcacttgggcagccccagcgcttaggagggacacggggatggcggggtgctggcacctctgcctgtagccatcctcactccccagtgctcccaggccctcctcagagcccagtttggaaactggcccagtcctgcctgtggggtttgggtggggaaactgaggctggagataaaaactggctagggaccgggtgcagtggctcacgtctataatctcggcactttgggaggccgaggtgcgcagatcacttgagaccaggagtttgagactagcctgccaacatggtgaaacccccgtctctactaaaaatataaaacttagctgggtgtggtggtgcacacctgtaatcccggctacttgggaggctgaggcacaagaatcgcttgaacccgagaggcagaggctgcaggagccgagattttaccactgcactccagccagggcgatagagcaagactccgtctcaaaaaaagaaaaacccggtggagagggccagtcctccttggggggcggagtcctgcttgggaggcggagtcctgcttggggggggcggagtcctgctggggggcggagtcctgctgggggttggagtccggttgtggggacagagtcctgctggggggctaagcctgggtgtcccccacgtccaagagggcttcgtgaccctgagtgtatgtggccagaccaggtcaagtggtggcctcctcagggctgtctctgggaaccccgacagctgtggaaccacgcttgtgaggctggcgggtgggtgaaagacgtggggtggagctcctgatctggtacaggcctgcagcctcacctgacctcctgcagggaggagtggacaaccgccatccagactgtggctgacggcctcaagaagcaggaggaggaggagatggacttccggtcgggctcacccagtgacaactcaggggctgaagagatggaggtgtccctggccaagcccaagcaccgcgtggtgaggcctgtccccacttctgcctgtgcctggggctgccttggactgtggagggctgggtgggtcatggggtcccagctcggcccaggagacactcagcactccacggcatctccacctaggccggttttccatgtagggcgcagctgcttcccaggtgggtcctcttccccatgtcagaggaaaactggggttgctgagccctgccaggcttgggcaatgttaccccacccttccctgaaccccatgctctgagctggggccgcagccatgtgttccctgtaagcctggactccccgctgagcccagtaggccaccaggtgtgaagtggacgcctctcctggctctgcctccgactctggggcccctcatgccaccccacgcatgccgaccagctgggcactgttggcagcgccgcttgctgaccctggtgcctgcccatagaccatgaacgagtttgagtacctgaagctgctgggcaagggcactttcggcaaggtgatcctggtgaaggagaaggccacaggccgctactacgccatgaagatcctcaagaaggaagtcatcgtggccaaggtggggccggggcggtggggcagggtggagatgagggtgcggggtggcagctcacccagccctgctttacaggacgaggtggcccacacactcaccgagaaccgcgtcctgcagaactccaggcaccccttcctcacagtgagtgggagcccagatggggctgaagggctggggccaggtagcgactcctggggctgtctctaggggacgatgtcctgtcttcttgggcacgtgagggctttacacaccgtggactccgccgctggtgagacaggcctgccctggagggtcaggctgtggaggtgcccagtgtgggctaggggctgtgctcgctgctcccgtgtgcgggcaggtgggtggggcaggtggccctggtcttccttgcactctgccgaggctccaggaggccccagctccccatgccctgcagccctgcctgccaggcttttgttgaggcccgggctctgtcctgcagaacctgttctggggacttggcccaggaggtatttactgtgaagggggccagggtccagccacttggttagttccaggtcactgtgtttggggacgattctcctgcagatggggcctttgctttaaaagcacagtcgcctgtggtggaaggcgggagggtgggtctcgcctgggagggtgtgtcctccctttgcgaaggccctggtgctgggagcactgtcccctgctgaggtcccaactcaccctggaggacctgagcccaggccaccttgctcacctttcaaacactccttggcacctcaggggctgccggggcccagcctgatggtggcgtggcatgaggcagtgtggtgtggggcagtgtggtgtggggcagcgtggcatgggggcagtgtgatgtgggggtgtatggtgtggggcagtgtgatggggtgtgtggtgtggggcagtgtggtatcgggcggtgtggtatcaggcagcgtggtgtcgggcagtgtggtgtggggctacgggcggtgtggtatcaggcgacgtggtatcaagcagcgtggtgtcgggcagcgtggtgcggggctgccgtcctgcctgtggggccgcagttccagctccccttccttgcaggccctgaagtactctttccagacccacgaccgcctctgctttgtcatggagtacgccaacgggggcgaggtaggggctggggctgcgggggatggacttcgcggcctgtgggccgcccatggtggggccggtccttcccggagcctcggctactctccatggcaccagacggtgctccctgctgggtgggtggtgtgatgcctgcccaggcagctgcctgctgtggggtgacttgttcctgctgagttagggcttctgagactttccaggcccccgtgccctctggtggctggtcccttccctgtgcaatgccgagacccggtctgagaaaccccagccctgcaggccatgggggcagaggcccgagccacggctgtgcctcaggttgcgcctcccgcagctgttcttccacctgtcccgggagcgtgtgttctccgaggaccgggcccgcttctatggcgctgagattgtgtcagccctggactacctgcactcggagaagaacgtggtgtaccgggacctcaaggtgcgctggcgggcaggcaggggggcagggccctgggggcctggcggcactgacctgaggccacctttcccctagctggagaacctcatgctggacaaggacgggcacattaagatcacagacttcgggctgtgcaaggaggggatcaaggacggtgccaccatgaagaccttttgcggcacacctgagtacctggcccccgaggtgtgcgccccacctgcgtgcatacgcgttgctgcgtccccacgtcctgagcacacgcaatgctgtgtcctctctgtgccccaagcacgtcacacctcccccggcagtgtcccggacaccccttgatgccgagtcctgcccatctgccacccgtgcaggtgctggaggacaatgactacggccgtgcagtggactggtgggggctgggcgtggtcatgtacgagatgatgtgcggtcgcctgcccttctacaaccaggaccatgagaagctttttgagctcatcctcatggaggagatccgcttcccgcgcacgcttggtcccgaggccaagtccttgctttcagggctgctcaagaaggaccccaagcagaggtgagggccgcccatcccagctacaggctacacctccatcccctcatccccaggctgcacctgcccctgcgccaggcggtcctggcacctcccagactacactgatagccaaagcttgatgtccttggccagggctgatctccaaagcctcaggcccaagcagagtggcgggcaggccggggtcagttgggcctctgtccccaaccctgcagcctgcacccacccactcaggaagcccctgccctgccgtgagctctgtggtgctttgctacccacagctgctcagggacgctgcaccaccggctcccctccctggccccggaacgtcctgtctggcgggccctacatcacaggaggaaggggcctgaacccagggcctgggcaggtggcggtaccgacactgtggccttgtttcctgcctgcaggcttggcgggggctccgaggacgccaaggagatcatgcagcatcgcttctttgccggtatcgtgtggcagcacgtgtacgagaagaaggtgcggctgctccccgcatattcacgcgcacgcatgctccccacatatccacactcacgcatgcacgtggcacgctcgccagatttcccacacactcgccctcacctcaggagcctgctgcagtcctggtacaaggagggccttgctgcaccaacctcagcgcctggtgctcagaggctctggcactgccgggttccaccaggaaactggcctggtcctcatttcctcctcccctcggaggtgtgtcacactctgagtttcctcctcccctcggaggtgtgtcacactctgagtgccagccttggggttcccttccctgacgctgtgcagtgaaggctggctggtggtggaccaggggtgctgccccttggcctccacgagttcctcctgtttgacctcaatcccttctgccgcaaggagagcccagccctatttctggctgtgcagggacagggacagcacctacttttctggcacatgggggagtccgccctggagggaggcagctctttctgcatgagtcgccatcctgggtgctcatctttcagggaccctaggagccctggccatcaccccagctgatggggtctgccagctgggttcggaagcctgcactctgagatgctgggtgccctgtcaccaggtgtgcttctgccccgttcccagcctgcccctcccctgaatgcctgtcctaggctgtgtgcagctggtcgggctgggcagccctgggacccagctgggcacctgttctcccattaacacagggtgccctggtcctggtccccgttcctgccttgaggcctgccaccctcctgccctgccctccaagggtatggggggcctgggcagtgctcaacctctgcccagccttgaccagagaccttgctaattgacaatggacaatgctggtgcccagcccagactctggggtggggcagtgcttgagggggtccttgactgtgggacctgaggggctctctggcagctcctcacgtgtgcacatcaccttatagtcacccttcatcctgggtcattgagagtcctgtctgcagccagatgccagcaggctgtggtcctgtagtccctgagccatggaggcagggtgggctcatcctgtgcaccttgttccacagtctccctctttcccaatttacaagaagaccaataagaaaaataagaaaacaagaaagaaaaaggaaaaataaagttatcacctcctggtagcagggagggtctctcagagctgggctgcactacctcctgtcgcctgctggggctgctgtgagggcagctttgcgtctcagctgttgagggttgtctccgtgctcctgcacccctcatgtccctccctcccagagctgcctctgtggggggctggtggcttgctgctctctgacatcagtcctgcctggagaccccttggagatccaggtgctttgaaggtcttgagcacacttgagggtgtgctgggagtggggagcgaagctcatgactgtcccgtctgcccacctctgcagctcagcccacccttcaagccccaggtcacgtcggagactgacaccaggtattttgatgaggagttcacggcccagatgatcaccatcacaccacctgaccaaggtgaggggccactgcctgcccccgcccactcccttttctctccacactcagagaggcctgcagtgttcagcttttttggcttcagatgttttaaaatctaaatatttaaaaaggttccttttggagagtgccaatgatcagggtgggaggcagtgctctgagggcccaggtccctgtgtcaatctgtggggccctgtctcagtttcctggcagtatggcagcgtgctggccgtgctttaagaggttggcttcctactggagctgtgggtgggtggaggtggcagggaggtggggcaccggggtctgagctgtctacacccacagatgacagcatggagtgtgtggacagcgagcgcaggccccacttcccccagttctcctactcggccagcggcacggcctgaggcggcggtggactgcgctggacgatagcttggagggatggagaggcggcctcgtgccatgatctgtatttaatggtttttatttctcgggtgcatttgagagaagccacgctgtcctctcgagcccagatggaaagacgtttttgtgctgtgggcagcaccctcccccgcagcggggtagggaagaaaactatcctgcgggttttaatttatttcatccagtttgttctccgggtgtggcctcagccctcagaacaatccgattcacgtagggaaatgttaaggacttctgcagctatgcgcaatgtggcattggggggccgggcaggtcctgcccatgtgtcccctcactctgtcagccagccgccctgggctgtctgtcaccagctatctgtcatctctctggggccctgggcctcagttcaacctggtggcaccagatgcaacctcactatggtatgctggccagcaccctctcctgggggtggcaggcacacagcagccccccagcactaaggccgtgtctctgaggacgtcatcggaggctgggcccctgggatgggaccagggatgggggatgggccagggtttacccagtgggacagaggagcaaggtttaaatttgttattgtgtattatgttgttcaaatgcattttgggggtttttaatctttgtgacaggaaagccctcccccttccccttctgtgtcacagttcttggtgactgtcccaccgggagcctccccctcagatgatctctccacggtagcacttgaccttttcgacgcttaacctttccgctgtcgccccaggccctccctgactccctgtgggggtggccatccctgggcccctccacgcctcctggccagacgctgccgctgccgctgcaccacggcgtttttttacaacattcaactttagtatttttactattataatataatatggaaccttccctccaaattcttcaataaaagttgcttttcaaatttttggctcactttgctgggtggaagagtgggtggccagggagggagtggggagtcctgggagggaagcagcatcctcagagcagctggtcctccccagctcctcctggtgggcaccgccaccccaacaggtttggggacaggctgtgtggtgggccgaggcgggagtatacaggcctgttcccaggggcagcctcccatggaaggtgttccacccccatctccagaggcctggcctgaggggagatgggaggccctggctggccctgcccccgttgggatccacctcttcttcccttgaccccacccagcttcaccccagccatgaaggagaccattggaccacgtggccatctgccccccagccccaggtgctgttcccctgggtagggcagtgaatgagacagaccaccaggaaggacagatagggcagggcccagagttggggccaaggtcatggcctgggccacggtggaagtaaggctgggaggcctgagtggcctgttcgaagctggacagagcgggaggaggggctgcaggcggtgggaggaggggctgcagggggcgggaggggacaggctggggcccaggttaccccagagcactacatcgagctctgagccggaacagcctgcagaatgtgccggtgctgccacctggtggacgcctgccggaaccgcgccctgagccaggagaggagtccactgccgagcaactgacagcgggttggcgaccaggttccgggtggttgggtgaccagcacgagccaggggcctggagcttcagcttcctcatctctcaaatggactccccgtaggtgtggatggttgcagagtcaggcccctggcgatggcaattggcccctctctaaggcattcccaggggtgcgtggctgccctcacttccactggagggggtgccccatagggagcacaggctggcgaggatgttctgggtctgtgggagccaagaacagccacatctggagcagcccgaagctctcctctgggtgatcccttattgcttcagttctaacaggccgcaaaactgataccgcttttcgggaaatacacgaaacatgacacagtaaatgatgttccgagggtgagaggcatctggatttcagatctatcaataatgtgactcttagaaaccaaaaaatacagcaatgcccttccaatatccacaaactttgagcaaagcattgctgaaagtaaccaaggtatggaaatggtcaggcagacgcagcatctcccagacagcccggctgagaagcatgggctgcttctcccagcacggccccctctgacaatataggattgccttagggccatttcttaccttttttatggtagagatagggtctcgctatgttgttcaggccggtcttgaactccgggcctcaagtgatcctcctgccttgtcctcccaaaacgttgggattataaggtgtgagccaccacacctggcctcttagggatagttctatagtccagagagagagaggcttcaacgtcattggcattaatacagacatgaccctgtgttatgtccccgttacaagtggctgaggacctaacaaaatgaacgcagaaatggagaccctctgagaatttcccgaccctcctagcagccccagacagaaccatctacagcaggaggcagcatcccaggagtggggtggagggtgtcagtgaggggtgagatgtgcaaggagatgcacatctcagagaggaaactccaggaggtgcaggtgcccttgcaaggggcagtccagtcctggccagcgaggccagagcgggtggagagggccaggatcctgcagccccaacagggcctgccgaacagcgagggctgtctggagaggttgtaacaggaatacgccaaccaagaggaagctctccttcctgtgtcagaggtaccccgaaggaaacaatgcataaagtgtccatttccaagacaaagtgcctaaaatcagcttaggtcagcaaactacggaa

(19) United States
(12) Patent Application Publication
(10) Pub. No.: US 2013/0331405 A1
(43) Pub. Date: Dec. 12, 2013

(54) PIK3CA Mutation Status and SASH1 Expression Predicts Synergy Between Lapatinib and an AKT Inhibitor in HER2 Positive Breast Cancer

(71) Applicants: James E. Korkola, West Linn, OR (US); Joe W. Gray, Lake Oswego, OR (US); Nora Bayani, Berkeley, CA (US)

(72) Inventors: James E. Korkola, West Linn, OR (US); Joe W. Gray, Lake Oswego, OR (US); Nora Bayani, Berkeley, CA (US)

(73) Assignee: THE REGENTS OF THE UNIVERSITY OF CALIFORNIA, Oakland, CA (US)

(21) Appl. No.: 13/494,918

(22) Filed: Jun. 12, 2012

(51) Int. Cl. C12Q 1/68

(52) U.S. Cl. CPC C12Q 1/6886
USPC 514/266.24; 435/6.12; 436/501

(57) ABSTRACT

Methods for identifying a cancer patient, such as a breast cancer patient, suitable for treatment with a 4-anilinoquinazoline kinase inhibitor, such as lapatinib, and an AKT inhibitor, comprising detecting modulated expression of HER2 (ERBB2) and SASH1 or protein encoded thereof and detecting PIK3CA mutation status. High levels of expression in HER2 and high levels of SASH1 and/or positive PIK3CA mutation status indicate a patient that is suitable for treatment with a 4-anilinoquinazoline kinase inhibitor, such as lapatinib and an AKT inhibitor.

### STATEMENT OF GOVERNMENTAL SUPPORT

The invention described was made with government support under Contract No. DE-AC02-05CH11231 awarded by the U.S. Department of Energy, under Work for Others Agreement No. LB06002417, and under Grant No. CA 126551 SPORE grant and Grant No. P50 CA58207 awarded by the National Institutes of Health. The government has certain rights in this invention.

### INCORPORATION OF SEQUENCE LISTING AND TABLES

The application includes and incorporates the attached sequence listing and Tables 1 and 2.

### FIELD OF THE INVENTION

This invention relates generally to genetic markers involved in the diagnosis and prognosis of ERBB2/HER2-positive cancer (HER2 positive), predicting patient response to specific therapeutic compounds and providing such therapy to patients predicted to benefit from such therapy.

### BACKGROUND OF THE INVENTION

HER2 amplification occurs in approximately 20% of all breast cancer patients. Therapeutic agents such as trastuzumab and lapatinib have been developed that target this alteration. Unfortunately, many patients with HER2 amplification are non-responsive to these drugs, or develop resistance to the drugs. Currently, patients who are non-responsive or develop resistance to Lapatinib are put onto experimental trials with other chemotherapeutic agents for treatment of advanced breast cancer.

Lapatinib is currently given to patients with metastatic HER2 positive breast cancer in combination with traditional chemotherapeutic agents such as paclitaxel.

Recent evidence suggests that activation of the PI3K-AKT pathway may occur following inhibition of HER2. This suggests that targeting this pathway in combination with HER2 inhibition may be synergistic and thus have therapeutic benefit.

### SUMMARY OF THE INVENTION

The invention provides for a method for identifying a HER2-positive cancer patient suitable for treatment with a 4-anilinoquinazoline kinase inhibitor Lapatinib and an AKT inhibitor, comprising: (a) obtaining the sequence of the PIK3CA gene, mRNA, or protein from a sample from a patient; and (b) identifying any mutations in the PIK3CA gene or protein in a sample from the patient as compared to the wild-type sequence found in SEQ ID NOs:3, 4, or 11, wherein a mutation in the PIK3CA gene, mRNA, or protein indicates the patient is suitable for treatment with the 4-anilinoquinazoline kinase inhibitor and an AKT inhibitor.

The method, further comprising (c) measuring the genomic copy number or expression level of a gene encoding HER2 in a sample from the patient, and (d) comparing the HER2 genomic copy number in the patient to normal copy number or expression level of the gene encoding HER2, the expression level of the gene encoding HER2 in a normal tissue sample or a reference expression level, or the average expression level of HER2 in a panel of normal cell lines or cancer cell lines, wherein an increase in the expression level of HER2 indicates the patient is suitable for treatment with the 4-anilinoquinazoline kinase inhibitor and an AKT inhibitor. In another embodiment, the method, (c) measuring the HER2 protein levels in a sample from the patient, and (d) comparing the HER2 protein levels from the sample to normal HER2 protein levels in a normal tissue sample or a reference protein level, or the average protein level of HER2 in a panel of normal cell lines or cancer cell lines, wherein an increase in the protein levels of HER2 indicates the patient is suitable for treatment with the 4-anilinoquinazoline kinase inhibitor and an AKT inhibitor. Patients identified by the present invention will respond to the synergistic treatment of cancer with the 4-anilinoquinazoline kinase inhibitor and an AKT inhibitor.

In some embodiments of the invention, a method for identifying a HER2-positive cancer patient suitable for treatment with a 4-anilinoquinazoline kinase inhibitor and an AKT inhibitor, comprising: (a) measuring the expression level of the SASH1 gene in a sample from the patient; and (b) comparing the expression level of said gene from the patient with the expression level of the gene in a normal tissue sample or a reference expression level (such as the average expression level of the gene in a cell line panel or a cancer cell or tumor panel, or the like), wherein an increase in the expression level of SASH1 indicates the patient is suitable for treatment with the 4-anilinoquinazoline kinase inhibitor and an AKT inhibitor.

In some embodiments of the invention, the patient identified as suitable for treatment with a combination of 4-anilinoquinazoline kinase inhibitor and an AKT inhibitor. In other embodiments, the 4-anilinoquinazoline kinase inhibitor and an AKT inhibitor are administered concurrently or sequentially.

In another embodiment, a method for identifying a HER2-positive cancer patient suitable for treatment with a 4-anilinoquinazoline kinase inhibitor and an AKT inhibitor, comprising: (a) obtaining the sequence of the PIK3CA gene from a sample from a patient; (b) identifying any mutations in the PIK3CA gene in a sample from the patient as compared to the wild-type sequence found in SEQ ID NOs:3, 4 or 11, (c) measuring the expression level of the SASH1 gene in the sample from the patient; and (d) comparing the expression level of said gene from the patient with the expression level of the gene in a normal tissue sample or a reference expression level (such as the average expression level of the gene in a cell line panel or a cancer cell or tumor panel, or the like), wherein both a mutation in the PIK3CA gene and an increase in the expression level of SASH1 indicates the patient is suitable for treatment with the 4-anilinoquinazoline kinase inhibitor and an AKT inhibitor.

The invention provides for a method of treating a cancer patient comprising (a) identifying a cancer patient who is suitable for treatment with a 4-anilinoquinazoline kinase inhibitor and an AKT inhibitor using a method of the present invention, and (b) administering a therapeutically effective amount of the 4-anilinoquinazoline kinase inhibitor and the AKT inhibitor to the patient. In some embodiments, a combination of the 4-anilinoquinazoline kinase inhibitor and an AKT inhibitor, and in other embodiments, the 4-anilinoquinazoline kinase inhibitor and an AKT inhibitor are administered concurrently or sequentially.

In some embodiments, the cancer is breast cancer and the cancer patient is a breast cancer patient. In certain embodiments, the breast cancer patient is an ERBB2-positive breast cancer patient.

### BRIEF DESCRIPTION OF THE DRAWINGS AND TABLES

The foregoing aspects and others will be readily appreciated by the skilled artisan from the following description of illustrative embodiments when read in conjunction with the accompanying drawings.

FIG. 1 shows representative examples of HER2 positive breast cancer cell lines subtypes and their response to an AKT inhibitor (triangle), lapatinib (square), or a combination of the two (diamond). Y-axis represents % growth inhibition, X-axis represents dose. Each graph is labeled with the cell line name and the PIK3CA mutation found.

FIG. 2 shows a heatmap showing synergy across nine different doses of a 1:1 combinations of Lapatinib+AKT inhibitor (light gray indicates significant synergy, black indicates additivity/no significant interaction, and dark gray indicates antagonism). Note synergistic interactions between the inhibitors in the cell lines with PI3K pathway mutations, while those without PI3K mutations show additivity or antagonism.

FIG. 3 shows relative SASH1 expression levels in breast cancer cell lines that show synergy with lapatinib+AKT ((HCC202; MDAMB361; HCC1954; SUM190PT; HCC1569)) compared to those that are not synergistic/antagonistic (AU565; MDAMB453; SKBR3; SUM225CWN; UACC812) with the combination. BT474 does not cluster with either group.

FIG. 4 shows a heat map which demonstrates synergy found between Lapatinib and the AKT inhibitor GSK690693 is dependent upon PI3K pathway status. Combination index values were calculated across all nine doses of drug and transformed to take into account significance (upper 95% confidence interval less than 1). Significant synergy is denoted as light gray indicates significant synergy, black indicates additivity/no significant interaction, and dark gray indicates antagonismin the heat map. Clustering of the data revealed that all the lines with similar synergistic responses had PI3K pathway mutations (cells with mutations in the PI3K pathway are denoted in red or blue). In contrast, all but two (BT474, MDAMB453) of the non-synergistic lines were wild-type for PI3K.

FIG. 5 shows a heatmap which demonstrates synergy found between Lapatinib and the AKT inhibitor GSK2141795 is dependent upon PI3K pathway status. Combination index values were calculated across all nine doses of drug and transformed to take into account significance (upper 95% confidence interval less than 1). Significant synergy is denoted as light gray indicates significant synergy, black indicates additivity/no significant interaction, and dark gray indicates antagonism in the heat map. Clustering of the data revealed that all the lines with similar synergistic responses had PI3K pathway mutations except one (cells with mutations in the PI3K pathway are denoted in red or blue). In contrast, all but two (BT474, HCC1569) of the non-synergistic lines were wild-type for PI3K.

FIG. 6 shows graphs showing that introduction of a PIK3CA mutation results in synergy in previously non-synergistic HER2+ cell line. A) Dose response of EFM192A cells (wild-type PI3K pathway) with laptinib (square), AKTi (GSK690693, triangle), or a combination of the two (diamond) does not result in significant synergism. B) Introduction of an H1047R PIK3CA mutation into EFM192A results in significant synergy when treated with the same drug combination as in A (points with significant synergism are indicated with asterisks).

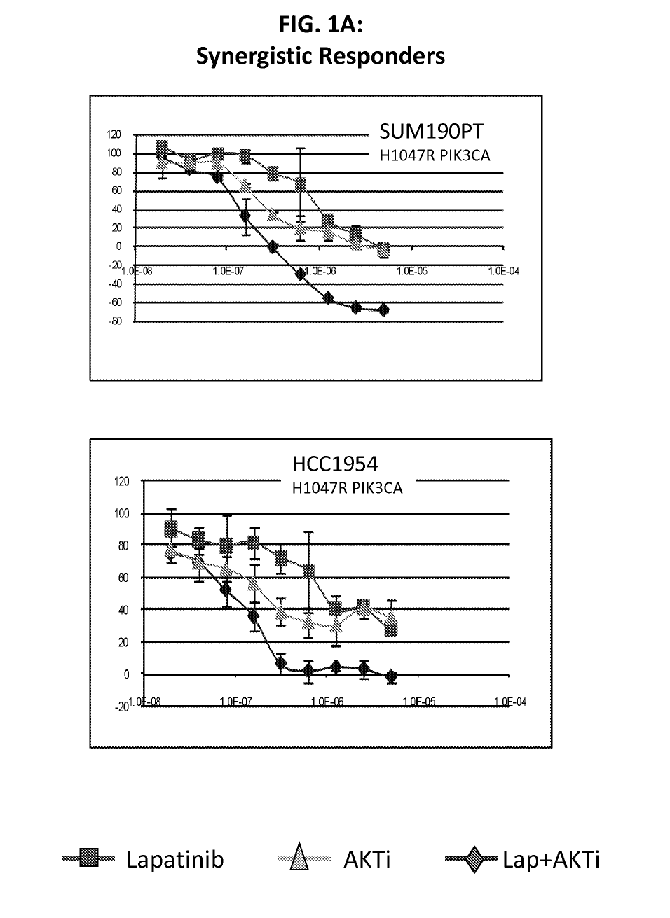

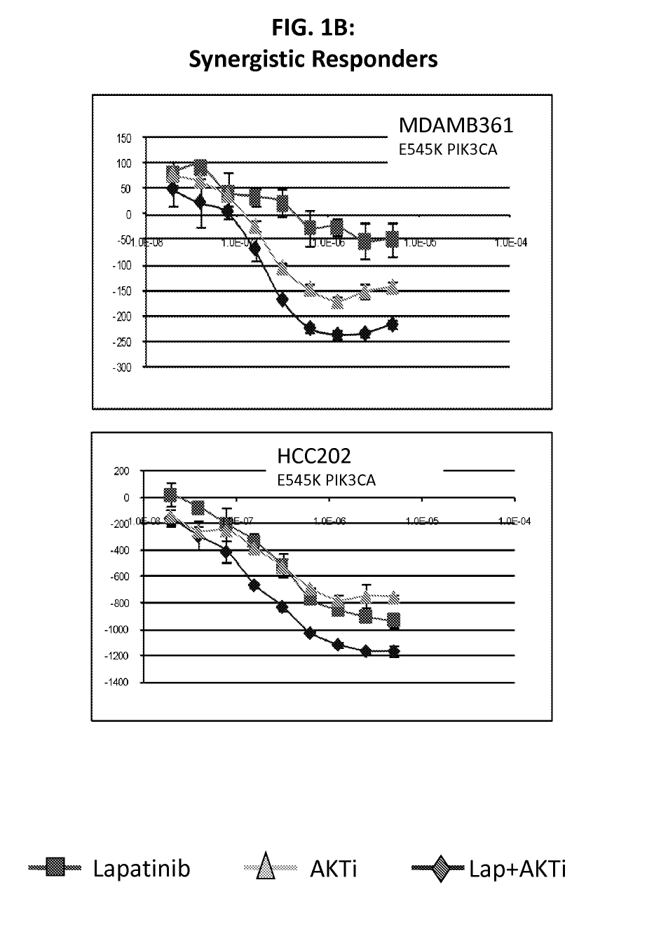

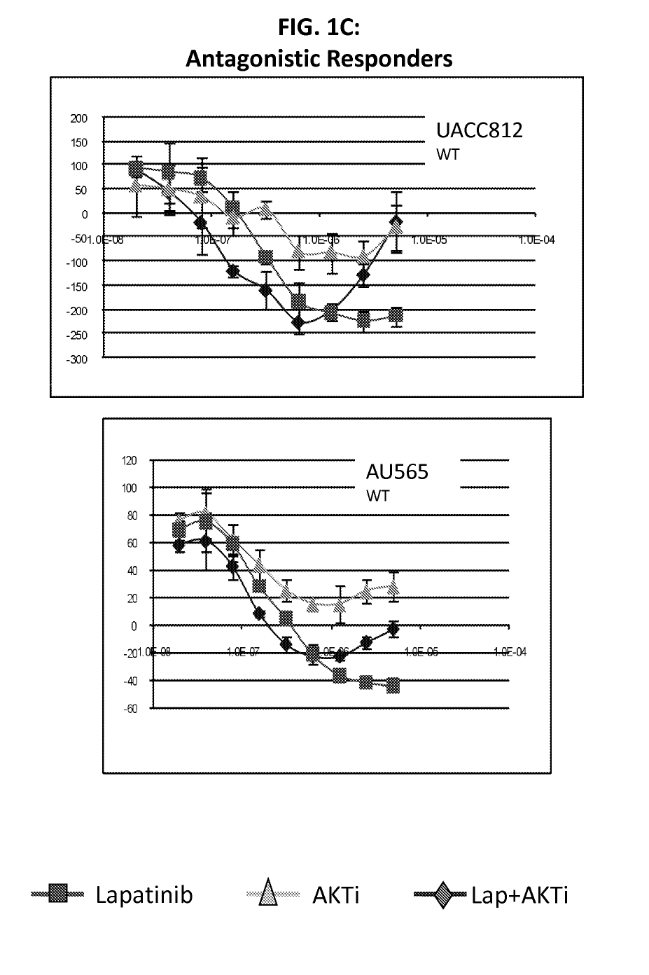

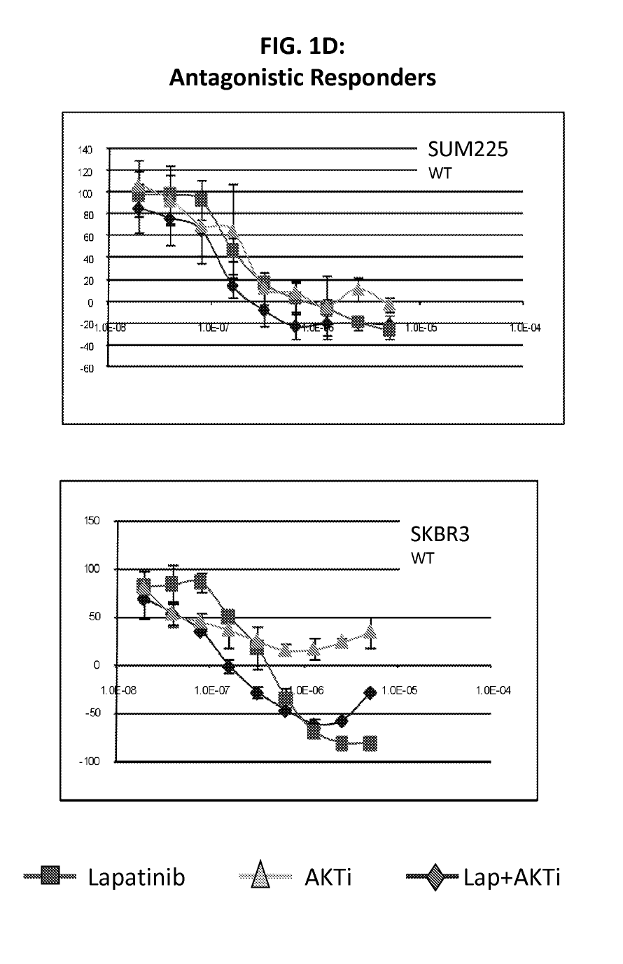

Table 1. Average GI50 values and combination indices at IC50 values to lapatinib, AKTi, or a combination of the two in HER2 positive cell lines with known PI3K mutation (either PIK3CA or PTEN; N=7) compared to those with known WT PI3K (N=5). Cell lines highlighted in bold (HCC202; MDAMB361; HCC1954; SUM190PT) show strongest synergistic interaction between Lapatinib and AKT.

Table 2. List of cell lines used in the Examples (note: MDAMB175 is not HER2 amplified and so was excluded from the synergy experiments).

### DETAILED DESCRIPTION

The present invention provides a new combination of targeted therapeutic agents that should have clinical efficacy for the treatment of HER2 positive breast cancer. The present invention also provides as genetic markers mutant PIK3CA and SASH1 which are herein shown to be predictive of patients most likely to respond to this combination.

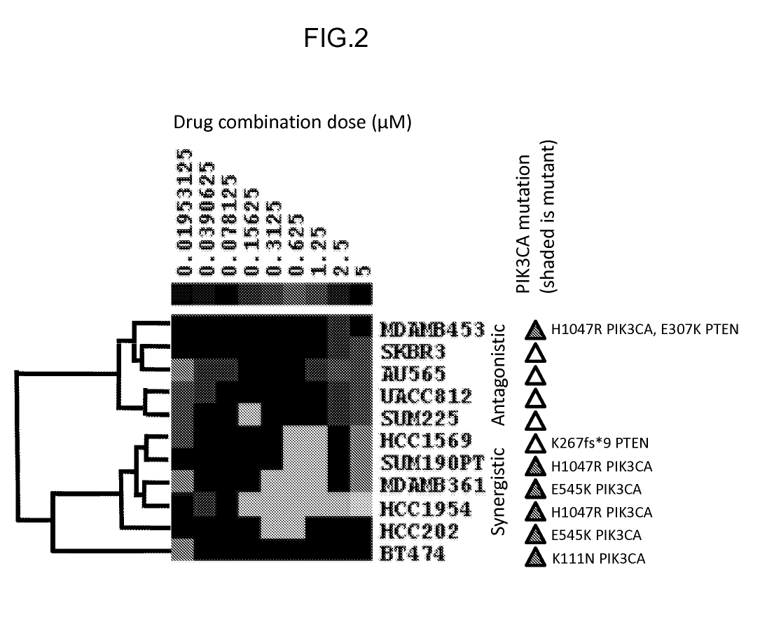

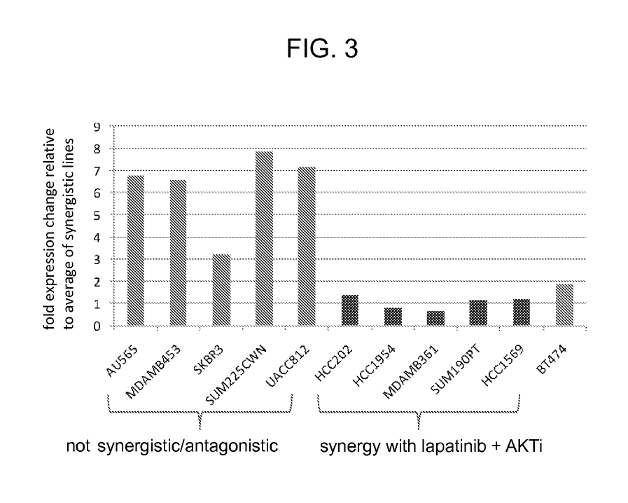

We have found and shown that a combination of Lapatinib with an AKT gene inhibitor is synergistic in HER2 positive patients who also have mutations in the PIK3CA gene and the PTEN gene (see FIG. 2). We also found that the combination could be antagonistic at high doses in patients with wild-type PIK3CA, indicating that careful patient selection will be required to ensure optimal patient populations for treatment with this drug combination. We also found that high levels of SASH1 gene expression predict response (and are correlated with PIK3CA mutation status). Thus, it represents an important advance by identifying both a new combination of targeted therapeutic agents with potential clinical efficacy and biomarkers that predict which patients are most likely to respond to this combination. This should result in improved treatment options for patients with HER2 positive cancer.

The cancer patient is either patient who is known to be ERBB2-positive, i.e., HER2-positive, and overexpresses the ERBB2 protein also known as the HER2 protein, or it is not known whether patient is ERBB2-positive or not. When the patient is not known whether to be ERBB2-positive or not, the ERBB2 status of the patient is to be determined.

The present methods describe the measurement and detection of expression levels of a gene as measured from a sample from a patient that comprises essentially a cancer cell or cancer tissue of a cancer tumor. Such methods for obtaining such samples are well known to those skilled in the art. When the cancer is breast cancer, the expression level of a gene is measured from a sample from the patient that comprises essentially a breast cancer cell or breast cancer tissue of a breast cancer tumor.

Methods for detection of expression levels of a gene can be carried out using known methods in the art including but not limited to, fluorescent in situ hybridization (FISH), immunohistochemical analysis, comparative genomic hybridization, PCR methods including real-time and quantitative PCR, and other sequencing and analysis methods. The expression level of the gene in question can be measured by measuring the amount or number of molecules of mRNA or transcript in a cell. The measuring can comprise directly measuring the mRNA or transcript obtained from a cell, or measuring the cDNA obtained from an mRNA preparation thereof. Such methods of extracting the mRNA or transcript from a cell, or preparing the cDNA thereof are well known to those skilled in the art. In other embodiments, the expression level of a gene can be measured by measuring or detecting the amount of protein or polypeptide expressed, such as measuring the amount of antibody that specifically binds to the protein in a dot blot or Western blot. The proteins described in the present invention can be overexpressed and purified or isolated to homogeneity and antibodies raised that specifically bind to each protein. Such methods are well known to those skilled in the art.

Comparison of the detected expression level of a gene in a patient sample is often compared to the expression levels detected in a normal tissue sample or a reference expression level. In some embodiments, the reference expression level can be the average or normalized expression level of the gene in a panel of normal cell lines or cancer cell lines.

In some embodiments, the method comprises measuring the expression level of ERBB2 of the patients in order to determine whether the patient is an ERBB2-positive patient. The expression level of a gene encoding ERBB2 can be measured using an oligonucleotide derived from the mouse v-erb-b2 erythroblastic leukemia viral oncogene homolog 2, neuro/glioblastoma derived oncogene homolog (avian) (Erbb2), mRNA sequence of GenBank Accession No. NM_{—}001003817.1 GI:54873609, hereby incorporated by reference and shown as SEQ ID NO: 1:

| | |
|---|---|
| 1 | cgaggaagtg cggcgtgaag ttgtggagct gagattgccc gccgctgggg acccggagcc |
| 61 | caggagcgcc ccttcccagg cggccccttc cggcgccgcg cctgtgcctg ccctcgccgc |
| 121 | gccccgcgcc cgcagcctgg tccagcctga gccatggggc cggagccgca gtgatcatca |
| 181 | tggagctggc ggcctggtgc cgttgggggt tcctcctcgc cctcctgtcc cccggagccg |
| 241 | cgggtaccca agtgtgtacc ggtaccgaca tgaagttgcg actccctgcc agtcctgaga |
| 301 | cccacctgga catgcttcgc cacctctacc agggctgtca ggtggtgcag ggcaatttgg |
| 361 | agcttaccta cctgcccgcc aatgccagcc tctcattcct gcaggacatc caggaagtcc |
| 421 | agggatacat gctcatcgct cacaaccgag tgaaacacgt cccactgcag aggttgcgca |
| 481 | tcgtgagagg gactcagctc tttgaggaca agtatgccct ggctgtgcta gacaaccgag |
| 541 | accctttgga caacgtcacc accgccgccc caggcagaac cccagaaggg ctgcgggagc |
| 601 | tgcagcttcg aagtctcaca gagatcttga agggaggagt tttgatccgt gggaaccctc |
| 661 | agctctgcta ccaggacatg gttttgtgga aggatgtcct ccgtaagaat aaccagctgg |
| 721 | ctcctgtcga catggacacc aatcgttccc gggcctgtcc accttgtgcc ccaacctgca |
| 781 | aagacaatca ctgttggggt gagagtcctg aagactgtca gatcttgact ggcaccatct |
| 841 | gtactagtgg ctgtgcccgg tgcaagggcc ggctgcccac tgactgttgc catgagcagt |
| 901 | gtgctgcagg ctgcacgggt cccaagcatt ctgactgcct ggcctgcctc cacttcaatc |
| 961 | atagtggtat ctgtgagctg cactgcccgg ccctcatcac ctacaacaca gacaccttcg |
| 1021 | agtccatgct caaccctgag ggtcgctaca cctttggtgc cagctgtgtg accacctgcc |
| 1081 | cctacaacta cctctccacg gaagtgggat cctgcactct ggtctgtccc ccgaacaacc |
| 1141 | aagaggtcac agctgaggac ggaacacagc ggtgtgagaa atgcagcaag ccctgtgctg |
| 1201 | gagtatgcta tggtctgggc atggagcacc tccgaggggc gagggccatc accagtgaca |
| 1261 | atatccagga gtttgctggc tgcaagaaga tctttgggag cctggcattt ttgccggaga |
| 1321 | gctttgatgg gaacccctcc tccggcgttg ccccactgaa gccagagcat ctccaagtgt |
| 1381 | tcgaaaccct ggaggagatc acaggttacc tatacatttc agcatggcca gagagcttcc |
| 1441 | aagacctcag tgtcttccag aaccttcggg tcattcgggg acggattctc catgatggtg |
| 1501 | cttactcatt gacgttgcaa ggcctgggga ttcactcact ggggctacgc tcactgcggg |
| 1561 | agctgggcag tggattggct ctcattcacc gcaacaccca tctctgcttt gtaaacactg |
| 1621 | taccttggga ccagctcttc cggaacccgc accaggccct actccacagt gggaaccggc |
| 1681 | cagaagaggc atgtggtctt gagggcttgg tctgtaactc actgtgtgcc cgtgggcact |
| 1741 | gctgggggcc agggcccacc cagtgtgtca actgcagtca gttcctccgg ggccaggagt |
| 1801 | gtgtggagga gtgccgagta tggaaggggc tccccaggga gtatgtgagg ggcaagcact |
| 1861 | gtctgccatg ccaccccgag tgtcagcctc aaaacagctc ggagacctgc tatggatcgg |
| 1921 | aggctgacca gtgtgaggct tgtgcccact acaaggactc atcttcctgt gtggctcgct |
| 1981 | gccccagtgg tgtgaagcca gacctctcct acatgcctat ctggaagtac ccggatgagg |
| 2041 | agggcatatg tcagccatgc cccatcaact gcacccactc atgtgtggac ctggacgaac |
| 2101 | gaggctgccc agcagagcag agagccagcc cagtgacatt catcattgca actgtggtgg |
| 2161 | gcgtcctgtt gttcctgatc atagtggtgg tcattggaat cctaatcaaa cgaaggcgac |
| 2221 | agaagatccg gaagtatacc atgcgtaggc tgctgcagga gaccgagctg gtggagccgc |
| 2281 | tgacgcccag tggagctgtg cccaaccagg ctcagatgcg gatcctaaag gagacagagc |
| 2341 | taaggaagct gaaggtgctt gggtcaggag ccttcggcac tgtctacaag ggcatctgga |
| 2401 | tcccagatgg ggagaacgtg aaaatccccg tggccatcaa ggtgttgagg gaaaacacat |
| 2461 | ctcctaaagc taacaaagaa atcctagatg aagcgtacgt catggctggt gtgggttctc |
| 2521 | catatgtgtc ccgcctcctg ggcatctgcc tgacatccac agtgcagctg gtgacacagc |
| 2581 | ttatgcccta tggctgcctt ctggaccatg tccgagaaca ccgaggtcgc ttaggctccc |
| 2641 | aggacctgct caactggtgt gttcagattg ccaaggggat gagctacctg gaggaagttc |
| 2701 | ggcttgttca cagggaccta gctgcccgaa acgtgctagt caagagtccc aaccacgtca |
| 2761 | agattaccga cttcgggctg gcacggctgc tggacattga tgagactgaa taccatgcag |
| 2821 | atgggggcaa ggtgcccatc aagtggatgg cattggaatc tattctcaga cgccggttca |
| 2881 | cccatcagag tgatgtgtgg agctatggtg tgactgtgtg ggagctgatg acctttgggg |
| 2941 | ccaaacctta cgatgggatc ccagctcggg agatccctga tttgctggag aagggagaac |
| 3001 | gcctacctca gcctccaatc tgcaccatcg acgtctacat gatcatggtc aaatgttgga |
| 3061 | tgattgactc cgaatgtcgc ccgagattcc gggagttggt atcagaattc tcccgtatgg |
| 3121 | caagggaccc ccagcgcttt gtggtcatcc agaacgagga cttaggcccc tccagcccca |
| 3181 | tggacagcac cttctaccgt tcactgctgg aggatgatga catgggggag ctggtcgatg |
| 3241 | ctgaagagta cctggtaccc cagcagggat tcttctcccc agaccctgcc ctaggtactg |
| 3301 | ggagcacagc ccaccgcaga caccgcagct cgtcggccag gagtggcggt ggtgagctga |
| 3361 | cactgggcct ggagccctcg gaagaagagc cccccagatc tccactggct ccctccgaag |
| 3421 | gggctggctc cgatgtgttt gatggtgacc tggcagtggg ggtaaccaaa ggactgcaga |
| 3481 | gcctctctcc acatgacctc agccctctac agcggtacag tgaggatccc acattacctc |
| 3541 | tgccccccga gactgatggc tacgttgctc ccctggcctg cagcccccag cccgagtatg |
| 3601 | tgaaccagcc agaggttcgg cctcagtctc ccttgacccc agagggtcct ccgcctccca |
| 3661 | tccgacctgc tggtgctact ctagaaagac ccaagactct ctctcctggg aaaaatgggg |
| 3721 | ttgtcaaaga cgtttttgcc tttgggggtg ctgtggagaa ccctgaatac ttagcaccca |
| 3781 | gagcaggcac tgcctctcag ccccaccctt ctcctgcctt cagcccagcc tttgacaacc |
| 3841 | tctattactg ggaccagaac tcatcggagc agggtcctcc accaagtacc tttgaaggga |
| 3901 | cccccactgc agagaaccct gagtacctag gcctggatgt gccagtatga ggtcacatgt |
| 3961 | gcagacatcc tctgtcttca gagtggggaa ggaaggccta acttgtggtc tccatcgccc |
| 4021 | gccacaaagc agggagaagg tcctctggcc acatgacatc cagggcagcc ggctatgcca |
| 4081 | ggaacgtgcc ctgaggaacc tcgctcgatg cttcaatcct gagtggttaa gagggccccg |
| 4141 | cctggccgga agagacagca cactgttcag ccccagagga ttacagaccc tgactgccct |
| 4201 | gacagactgt agggtccagt gggtattcct tacctggcct ggctctcttg gttctgaaga |
| 4261 | ctgagggaag ctcagcctgc aagggaggag gccccaggtg aatatcctgg gagcaggaca |
| 4321 | ccccactagg actgaggcac gtgcatccca agagggggac agcacttgca cccagactgg |
| 4381 | tctttgtaca gagtttattt tgttctgttt ttacttttgt tttttgtttt ttttttaaag |
| 4441 | atgaaataag gatacagtgg gagagtgggt gttatatgaa agtcgggggg tgctgtcccc |
| 4501 | tttctccatt tgcaatgaga tttgtaaaat aactggaccc cagcctatgt ctgagagtgg |
| 4561 | tcccgggccg ggtcaaaccg tattgctcat ctgacacaca gctcctcctg gagtgagtgt |
| 4621 | gtagagatct tccaaaagtt tgagacaatt tggctttggg cttgagggac tggggagtta |
| 4681 | ggattccttc tgaaggccct ttggcaacag ggtcattctc cgttggacac actcatacca |
| 4741 | aggctacccc cagaatactc cgttggacac actcattcca aggctacccc cagaatgaag |
| 4801 | tcctgtcctc ccagtgggag aggggagctt gtggagagca ttgccatgtg acttgttttc |
| 4861 | cttgccttag aaagaagtat ccatccagga aaaccccacc cactaggtgt tagtcccacc |
| 4921 | cactaggtgt tagcagggcc agactgacct gtgtgccccc cgcacaggct ggacataaac |
| 4981 | acacgccagt tgacacaa |

The expression level of a gene encoding ERBB2 can also be measured using an oligonucleotide derived from the human nucleotide sequence of GenBank Accession No. NM_{—}004448.2 GI:54792095, Homo sapiens v-erb-b2 erythroblastic leukemia viral oncogene homolog 2, neuro/glioblastoma derived oncogene homolog (avian) (ERBB2), transcript variant 1, mRNA, hereby incorporated by reference and shown as SEQ ID NO: 2:

| | |
|---|---|
| 1 | ggaggaggtg gaggaggagg gctgcttgag gaagtataag aatgaagttg tgaagctgag |
| 61 | attcccctcc attgggaccg gagaaaccag gggagccccc cgggcagccg cgcgcccctt |
| 121 | cccacggggc cctttactgc gccgcgcgcc cggcccccac ccctcgcagc accccgcgcc |
| 181 | ccgcgccctc ccagccgggt ccagccggag ccatggggcc ggagccgcag tgagcaccat |
| 241 | ggagctggcg gccttgtgcc gctgggggct cctcctcgcc ctcttgcccc ccggagccgc |
| 301 | gagcacccaa gtgtgcaccg gcacagacat gaagctgcgg ctccctgcca gtcccgagac |
| 361 | ccacctggac atgctccgcc acctctacca gggctgccag gtggtgcagg gaaacctgga |
| 421 | actcacctac ctgcccacca atgccagcct gtccttcctg caggatatcc aggaggtgca |
| 481 | gggctacgtg ctcatcgctc acaaccaagt gaggcaggtc ccactgcaga ggctgcggat |
| 541 | tgtgcgaggc acccagctct ttgaggacaa ctatgccctg gccgtgctag acaatggaga |
| 601 | cccgctgaac aataccaccc ctgtcacagg ggcctcccca ggaggcctgc gggagctgca |
| 661 | gcttcgaagc ctcacagaga tcttgaaagg aggggtcttg atccagcgga acccccagct |
| 721 | ctgctaccag gacacgattt tgtggaagga catcttccac aagaacaacc agctggctct |
| 781 | cacactgata gacaccaacc gctctcgggc ctgccacccc tgttctccga tgtgtaaggg |
| 841 | ctcccgctgc tggggagaga gttctgagga ttgtcagagc ctgacgcgca ctgtctgtgc |
| 901 | cggtggctgt gcccgctgca aggggccact gcccactgac tgctgccatg agcagtgtgc |
| 961 | tgccggctgc acgggcccca agcactctga ctgcctggcc tgcctccact tcaaccacag |
| 1021 | tggcatctgt gagctgcact gcccagccct ggtcacctac aacacagaca cgtttgagtc |
| 1081 | catgcccaat cccgagggcc ggtatacatt cggcgccagc tgtgtgactg cctgtcccta |
| 1141 | caactacctt tctacggacg tgggatcctg caccctcgtc tgccccctgc acaaccaaga |
| 1201 | ggtgacagca gaggatggaa cacagcggtg tgagaagtgc agcaagccct gtgcccgagt |
| 1261 | gtgctatggt ctgggcatgg agcacttgcg agaggtgagg gcagttacca gtgccaatat |
| 1321 | ccaggagttt gctggctgca agaagatctt tgggagcctg gcatttctgc cggagagctt |
| 1381 | tgatggggac ccagcctcca acactgcccc gctccagcca gagcagctcc aagtgtttga |
| 1441 | gactctggaa gagatcacag gttacctata catctcagca tggccggaca gcctgcctga |
| 1501 | cctcagcgtc ttccagaacc tgcaagtaat ccggggacga attctgcaca atggcgccta |
| 1561 | ctcgctgacc ctgcaagggc tgggcatcag ctggctgggg ctgcgctcac tgagggaact |
| 1621 | gggcagtgga ctggccctca tccaccataa cacccacctc tgcttcgtgc acacggtgcc |
| 1681 | ctgggaccag ctctttcgga acccgcacca agctctgctc cacactgcca accggccaga |
| 1741 | ggacgagtgt gtgggcgagg gcctggcctg ccaccagctg tgcgcccgag ggcactgctg |
| 1801 | gggtccaggg cccacccagt gtgtcaactg cagccagttc cttcggggcc aggagtgcgt |
| 1861 | ggaggaatgc cgagtactgc aggggctccc cagggagtat gtgaatgcca ggcactgttt |
| 1921 | gccgtgccac cctgagtgtc agccccagaa tggctcagtg acctgttttg gaccggaggc |
| 1981 | tgaccagtgt gtggcctgtg cccactataa ggaccctccc ttctgcgtgg cccgctgccc |
| 2041 | cagcggtgtg aaacctgacc tctcctacat gcccatctgg aagtttccag atgaggaggg |
| 2101 | cgcatgccag ccttgcccca tcaactgcac ccactcctgt gtggacctgg atgacaaggg |
| 2161 | ctgccccgcc gagcagagag ccagccctct gacgtccatc atctctgcgg tggttggcat |
| 2221 | tctgctggtc gtggtcttgg gggtggtctt tgggatcctc atcaagcgac ggcagcagaa |
| 2281 | gatccggaag tacacgatgc ggagactgct gcaggaaacg gagctggtgg agccgctgac |
| 2341 | acctagcgga gcgatgccca accaggcgca gatgcggatc ctgaaagaga cggagctgag |
| 2401 | gaaggtgaag gtgcttggat ctggcgcttt tggcacagtc tacaagggca tctggatccc |
| 2461 | tgatggggag aatgtgaaaa ttccagtggc catcaaagtg ttgagggaaa acacatcccc |
| 2521 | caaagccaac aaagaaatct tagacgaagc atacgtgatg gctggtgtgg gctccccata |
| 2581 | tgtctcccgc cttctgggca tctgcctgac atccacggtg cagctggtga cacagcttat |
| 2641 | gccctatggc tgcctcttag accatgtccg ggaaaaccgc ggacgcctgg gctcccagga |
| 2701 | cctgctgaac tggtgtatgc agattgccaa ggggatgagc tacctggagg atgtgcggct |
| 2761 | cgtacacagg gacttggccg ctcggaacgt gctggtcaag agtcccaacc atgtcaaaat |
| 2821 | tacagacttc gggctggctc ggctgctgga cattgacgag acagagtacc atgcagatgg |
| 2881 | gggcaaggtg cccatcaagt ggatggcgct ggagtccatt ctccgccggc ggttcaccca |
| 2941 | ccagagtgat gtgtggagtt atggtgtgac tgtgtgggag ctgatgactt ttggggccaa |
| 3001 | accttacgat gggatcccag cccgggagat ccctgacctg ctggaaaagg gggagcggct |
| 3061 | gccccagccc cccatctgca ccattgatgt ctacatgatc atggtcaaat gttggatgat |
| 3121 | tgactctgaa tgtcggccaa gattccggga gttggtgtct gaattctccc gcatggccag |
| 3181 | ggacccccag cgctttgtgg tcatccagaa tgaggacttg ggcccagcca gtcccttgga |
| 3241 | cagcaccttc taccgctcac tgctggagga cgatgacatg ggggacctgg tggatgctga |
| 3301 | ggagtatctg gtaccccagc agggcttctt ctgtccagac cctgccccgg gcgctggggg |
| 3361 | catggtccac cacaggcacc gcagctcatc taccaggagt ggcggtgggg acctgacact |
| 3421 | agggctggag ccctctgaag aggaggcccc caggtctcca ctggcaccct ccgaaggggc |
| 3481 | tggctccgat gtatttgatg gtgacctggg aatgggggca gccaaggggc tgcaaagcct |
| 3541 | ccccacacat gaccccagcc ctctacagcg gtacagtgag gaccccacag tacccctgcc |
| 3601 | ctctgagact gatggctacg ttgcccccct gacctgcagc ccccagcctg aatatgtgaa |
| 3661 | ccagccagat gttcggcccc agcccccttc gccccgagag ggccctctgc ctgctgcccg |
| 3721 | acctgctggt gccactctgg aaaggcccaa gactctctcc ccagggaaga atggggtcgt |
| 3781 | caaagacgtt tttgcctttg ggggtgccgt ggagaacccc gagtacttga caccccaggg |
| 3841 | aggagctgcc cctcagcccc accctcctcc tgccttcagc ccagccttcg acaacctcta |
| 3901 | ttactgggac caggacccac cagagcgggg ggctccaccc agcaccttca aagggacacc |
| 3961 | tacggcagag aacccagagt acctgggtct ggacgtgcca gtgtgaacca gaaggccaag |
| 4021 | tccgcagaag ccctgatgtg tcctcaggga gcagggaagg cctgacttct gctggcatca |
| 4081 | agaggtggga gggccctccg accacttcca ggggaacctg ccatgccagg aacctgtcct |
| 4141 | aaggaacctt ccttcctgct tgagttccca gatggctgga aggggtccag cctcgttgga |
| 4201 | agaggaacag cactggggag tctttgtgga ttctgaggcc ctgcccaatg agactctagg |
| 4261 | gtccagtgga tgccacagcc cagcttggcc ctttccttcc agatcctggg tactgaaagc |
| 4321 | cttagggaag ctggcctgag aggggaagcg gccctaaggg agtgtctaag aacaaaagcg |
| 4381 | acccattcag agactgtccc tgaaacctag tactgccccc catgaggaag gaacagcaat |
| 4441 | ggtgtcagta tccaggcttt gtacagagtg cttttctgtt tagtttttac tttttttgtt |
| 4501 | ttgttttttt aaagatgaaa taaagaccca gggggagaat gggtgttgta tggggaggca |
| 4561 | agtgtggggg gtccttctcc acacccactt tgtccatttg caaatatatt ttggaaaaca |
| 4621 | gcta |

Methods of assaying for ERBB2 or HER2 protein overexpression include methods that utilize immunohistochemistry (1HC) and methods that utilize fluorescence in situ hybridization (FISH). A commercially available IHC test is DAKO HercepTest® (DAKO Corp., Carpinteria, Calif.). Patient samples having an IHC staining score of 0-1, 2 is normal, and scores of 2+ may be borerderline, while results of 2, 3+ are scored as positive for multiple copies of HER2 (HER2 positive).

A commercially available FISH test is PathVysion® (Vysis Inc., Downers Grove, Ill.). The HER2 genomic copy number of a patient sample is determined using FISH. Generally if a sample is found to have 3.6 or more copies of HER2 (normal=2 copies), the patient is determined to be HER2 positive.

While many HER2-positive patients suffer from metastatic breast cancer, a patient's HER2 status can also be determined in relation to other types of cancers including but not limited to epithelial cancers such as pancreatic, lung, cervical, ovarian, prostate, non-small cell lung carcinomas, melanomas, squamous cell cancers, etc. It is contemplated that the present methods described herein may find use in prognosis and predicting patient response to certain combination therapies that may be used in various cancer treatments for multiple types of cancers so long as the patient criteria described herein is present as identifying a patient suitable for such combination therapy.

In one embodiment a method for identifying a HER2-positive cancer patient suitable for treatment with a 4-anilinoquinazoline kinase inhibitor and an AKT inhibitor, comprising: (a) obtaining the sequence of the PIK3CA gene from a sample from a patient; and (b) identifying any mutations in the PIK3CA gene in a sample from the patient as compared to the wild-type sequence found in SEQ ID NO:1, wherein a mutation in the PIK3CA gene indicates the patient is suitable for treatment with the 4-anilinoquinazoline kinase inhibitor and an AKT inhibitor.

The PIK3CA gene is identified as Homo sapiens phosphoinositide-3-kinase, catalytic, alpha polypeptide (PIK3CA) on chromosome 3, and the wild-type PIK3CA gene sequence is found at GenBankAccession No. NG 012113.1 GI:237858742, hereby incorporated by reference and identified as SEQ ID NO:11.

The PIK3CA mRNA sequence is found at NM_{—}006218.2 GI:54792081, hereby incorporated by reference, and shown here as SEQ ID NO:3.

| | |
|---|---|
| 1 | tctccctcgg cgccgccgcc gccgcccgcg gggctgggac ccgatgcggt tagagccgcg |
| 61 | gagcctggaa gagccccgag cgtttctgct ttgggacaac catacatcta attccttaaa |
| 121 | gtagttttat atgtaaaact tgcaaagaat cagaacaatg cctccacgac catcatcagg |
| 181 | tgaactgtgg ggcatccact tgatgccccc aagaatccta gtagaatgtt tactaccaaa |
| 241 | tggaatgata gtgactttag aatgcctccg tgaggctaca ttaataacca taaagcatga |
| 301 | actatttaaa gaagcaagaa aataccccct ccatcaactt cttcaagatg aatcttctta |
| 361 | cattttcgta agtgttactc aagaagcaga aagggaagaa ttttttgatg aaacaagacg |
| 421 | actttgtgac cttcggcttt ttcaaccctt tttaaaagta attgaaccag taggcaaccg |
| 481 | tgaagaaaag atcctcaatc gagaaattgg ttttgctatc ggcatgccag tgtgtgaatt |
| 541 | tgatatggtt aaagatccag aagtacagga cttccgaaga aatattctga acgtttgtaa |
| 601 | agaagctgtg gatcttaggg acctcaattc acctcatagt agagcaatgt atgtctatcc |
| 661 | tccaaatgta gaatcttcac cagaattgcc aaagcacata tataataaat tagataaagg |
| 721 | gcaaataata gtggtgatct gggtaatagt ttctccaaat aatgacaagc agaagtatac |
| 781 | tctgaaaatc aaccatgact gtgtaccaga acaagtaatt gctgaagcaa tcaggaaaaa |
| 841 | aactcgaagt atgttgctat cctctgaaca actaaaactc tgtgttttag aatatcaggg |
| 901 | caagtatatt ttaaaagtgt gtggatgtga tgaatacttc ctagaaaaat atcctctgag |
| 961 | tcagtataag tatataagaa gctgtataat gcttgggagg atgcccaatt tgatgttgat |
| 1021 | ggctaaagaa agcctttatt ctcaactgcc aatggactgt tttacaatgc catcttattc |
| 1081 | cagacgcatt tccacagcta caccatatat gaatggagaa acatctacaa aatccctttg |
| 1141 | ggttataaat agtgcactca gaataaaaat tctttgtgca acctacgtga atgtaaatat |
| 1201 | tcgagacatt gataagatct atgttcgaac aggtatctac catggaggag aacccttatg |
| 1261 | tgacaatgtg aacactcaaa gagtaccttg ttccaatccc aggtggaatg aatggctgaa |
| 1321 | ttatgatata tacattcctg atcttcctcg tgctgctcga ctttgccttt ccatttgctc |
| 1381 | tgttaaaggc cgaaagggtg ctaaagagga acactgtcca ttggcatggg gaaatataaa |
| 1441 | cttgtttgat tacacagaca ctctagtatc tggaaaaatg gctttgaatc tttggccagt |
| 1501 | acctcatgga ttagaagatt tgctgaaccc tattggtgtt actggatcaa atccaaataa |
| 1561 | agaaactcca tgcttagagt tggagtttga ctggttcagc agtgtggtaa agttcccaga |
| 1621 | tatgtcagtg attgaagagc atgccaattg gtctgtatcc cgagaagcag gatttagcta |
| 1681 | ttcccacgca ggactgagta acagactagc tagagacaat gaattaaggg aaaatgacaa |
| 1741 | agaacagctc aaagcaattt ctacacgaga tcctctctct gaaatcactg agcaggagaa |
| 1801 | agattttcta tggagtcaca gacactattg tgtaactatc cccgaaattc tacccaaatt |
| 1861 | gcttctgtct gttaaatgga attctagaga tgaagtagcc cagatgtatt gcttggtaaa |
| 1921 | agattggcct ccaatcaaac ctgaacaggc tatggaactt ctggactgta attacccaga |
| 1981 | tcctatggtt cgaggttttg ctgttcggtg cttggaaaaa tatttaacag atgacaaact |
| 2041 | ttctcagtat ttaattcagc tagtacaggt cctaaaatat gaacaatatt tggataactt |
| 2101 | gcttgtgaga tttttactga agaaagcatt gactaatcaa aggattgggc actttttctt |
| 2161 | ttggcattta aaatctgaga tgcacaataa aacagttagc cagaggtttg gcctgctttt |
| 2221 | ggagtcctat tgtcgtgcat gtgggatgta tttgaagcac ctgaataggc aagtcgaggc |
| 2281 | aatggaaaag ctcattaact taactgacat tctcaaacag gagaagaagg atgaaacaca |
| 2341 | aaaggtacag atgaagtttt tagttgagca aatgaggcga ccagatttca tggatgctct |
| 2401 | acagggcttt ctgtctcctc taaaccctgc tcatcaacta ggaaacctca ggcttgaaga |
| 2461 | gtgtcgaatt atgtcctctg caaaaaggcc actgtggttg aattgggaga acccagacat |
| 2521 | catgtcagag ttactgtttc agaacaatga gatcatcttt aaaaatgggg atgatttacg |
| 2581 | gcaagatatg ctaacacttc aaattattcg tattatggaa aatatctggc aaaatcaagg |
| 2641 | tcttgatctt cgaatgttac cttatggttg tctgtcaatc ggtgactgtg tgggacttat |
| 2701 | tgaggtggtg cgaaattctc acactattat gcaaattcag tgcaaaggcg gcttgaaagg |
| 2761 | tgcactgcag ttcaacagcc acacactaca tcagtggctc aaagacaaga acaaaggaga |
| 2821 | aatatatgat gcagccattg acctgtttac acgttcatgt gctggatact gtgtagctac |
| 2881 | cttcattttg ggaattggag atcgtcacaa tagtaacatc atggtgaaag acgatggaca |
| 2941 | actgtttcat atagattttg gacacttttt ggatcacaag aagaaaaaat ttggttataa |
| 3001 | acgagaacgt gtgccatttg ttttgacaca ggatttctta atagtgatta gtaaaggagc |
| 3061 | ccaagaatgc acaaagacaa gagaatttga gaggtttcag gagatgtgtt acaaggctta |
| 3121 | tctagctatt cgacagcatg ccaatctctt cataaatctt ttctcaatga tgcttggctc |
| 3181 | tggaatgcca gaactacaat cttttgatga cattgcatac attcgaaaga ccctagcctt |
| 3241 | agataaaact gagcaagagg ctttggagta tttcatgaaa caaatgaatg atgcacatca |
| 3301 | tggtggctgg acaacaaaaa tggattggat cttccacaca attaaacagc atgcattgaa |
| 3361 | ctgaaaagat aactgagaaa atgaaagctc actctggatt ccacactgca ctgttaataa |
| 3421 | ctctcagcag gcaaagaccg attgcatagg aattgcacaa tccatgaaca gcattagaat |
| 3481 | ttacagcaag aacagaaata aaatactata taatttaaat aatgtaaacg caaacagggt |
| 3541 | ttgatagcac ttaaactagt tcatttcaaa attaagcttt agaataatgc gcaatttcat |
| 3601 | gttatgcctt aagtccaaaa aggtaaactt tgaagattgt ttgtatcttt ttttaaaaaa |
| 3661 | caaaacaaaa caaaaatccc caaaatatat agaaatgatg gagaaggaaa aaaaaaaaaa |
| 3721 | aaaa |
| // | |

The PIK3CA mRNA is expressed as the phosphatidylinositol-4,5-bisphosphate 3-kinase catalytic subunit alpha isoform [Homo sapiens](PI3_K or PIK3CA) protein having GenBank Accession No. NP 006209.2 GI:54792082, hereby incorporated by reference, and shown here as SEQ ID NO:4.

| | |
|---|---|
| 1 | mpprpssgel wgihlmppri lvecllpngm ivtleclrea tlitikhelf kearkyplhq |
| 61 | llqdessyif vsvtqeaere effdetrrlc dlrlfqpflk viepvgnree kilnreigfa |
| 121 | igmpvcefdm vkdpevqdfr rnilnvckea vdlrdlnsph sramyvyppn vesspelpkh |
| 181 | iynkldkgqi ivviwvivsp nndkqkytlk inhdcvpeqv iaeairkktr smllsseqlk |
| 241 | lcvleyqgky ilkvcgcdey flekyplsqy kyirscimlg rmpnlmlmak eslysqlpmd |
| 301 | cftmpsysrr istatpymng etstkslwvi nsalrikilc atyvnvnird idkiyvrtgi |
| 361 | yhggeplcdn vntqrvpcsn prwnewlnyd iyipdlpraa rlclsicsvk grkgakeehc |
| 421 | plawgninlf dytdtivsgk malnlwpvph gledllnpig vtgsnpnket pclelefdwf |
| 481 | ssvvkfpdms vieehanwsv sreagfsysh aglsnrlard nelrendkeq lkaistrdpl |
| 541 | seiteqekdf lwshrhycvt ipeilpklll svkwnsrdev aqmyclvkdw ppikpeqame |
| 601 | lldcnypdpm vrgfavrcle kyltddklsq yliqlvqvlk yeqyldnllv rfllkkaltn |
| 661 | qrighfffwh lksemhnktv sqrfgllles ycracgmylk hlnrqveame klinltdilk |
| 721 | qekkdetqkv qmkflveqmr rpdfmdalqg flspinpahq lgnlrleecr imssakrplw |
| 781 | lnwenpdims ellfqnneii fkngddlrqd mltlqiirim eniwqnqgld lrmlpygcls |
| 841 | igdcvgliev vrnshtimqi qckgglkgal qfnshtlhqw lkdknkgeiy daaidlftrs |
| 901 | cagycvatfi lgigdrhnsn imvkddgqlf hidfghfldh kkkkfgykre rvpfvltqdf |
| 961 | liviskgaqe ctktreferf qemcykayla irqhanlfin lfsmmlgsgm pelqsfddia |
| 1021 | yirktlaldk teqealeyfm kqmndahhgg wttkmdwifh tikqhaln |
| // | |

Missense mutations are most commonly found in two hotspot locations in PIK3CA, occurring in either the helical domain (amino acids 542 545) or the kinase domain (amino acids 1047 and 1049). See Saal et al. Cancer Research 65, 2554-2559, Apr. 1, 2005, “PIK3CA Mutations Correlate with Hormone Receptors, Node Metastasis, and ERBB2, and Are Mutually Exclusive with PTEN Loss in Human Breast Carcinoma,” hereby incorporated by reference. PIK3CA missense mutations detected by Saal et al. and others have found the following substitutions: E542K, E545K, H1047R, H1047L, H1047Y and G1049R. Furthermore, tumors with both PIK3CA copy number gain and mutation had moderate levels of gain and were enriched for E545K and other non-H1047R mutations.

Table 1 shows the various mutations found in the cell lines tested and include: E545K, H1047R, K111N, E307K. Cell lines having these mutations were found to have synergistic response. Either mutation is thought to be sufficient to activate the PI3K pathway, and both of these mutations have been shown to be transforming in vitro. Mutation of the PIK3CA gene is one of the most common mutations found in breast cancer, with mutation rates exceeding 20%. While cell lines from patients with the 542/545 mutations appear to be most resistant and those with 1047 mutation appear to be most sensitive to lapatinib as a monotherapy, both types appear to have comparable synergistic responses to the combination of lapatinib plus an AKT inhibitor.

Thus, in one embodiment, a biopsy, tissue or fluid sample is obtained from a patient and the mutation status of a patient at the PIK3CA gene is obtained by methods known and used the in the art. The two main PIK3CA mutation sites: aa 542-545 (E542K, E545K) and aa 1047 (H1047R, H1047L) correspond to PIK3CA mRNA sequence sites of 1624 bp, 1633, 1634 and 3140 bp. For example, in one embodiment, the patient's PIK3CA gene or mRNA is amplified and sequenced to identify any mutations in the PIK3CA gene. It may be preferable to use primers within the PIK3CA gene or mRNA sequence that are near the two hotspot locations for PIK3CA mutation to use to amplify those regions and then the resulting PCR products sequenced.

Other methods that can identify mutations in the candidate gene can be used. In one embodiment, the PIK3CA mutation in the PIK3CA protein expressed in the patient's tissue is identified. For example, immunohistochemical analysis by utilizing an antibody that specifically identifies an E545K or an H1047R mutation can be performed.

Two cell lines having a mutation in the PTEN gene were found. MDAMB453 features a E307 K mutation in PTEN and showed synergistic response to both lapatinib and the AKT inhibitor. Another cell line HCC1569 with a PTEN mutation showed antagonistic response to the combination therapy. It may be useful to identify the mutation status of a patient at the PTEN gene.

The PTEN gene was identified as a tumor suppressor that is mutated in a large number of cancers at high frequency. The protein encoded this gene is a phosphatidylinositol-3,4,5-trisphosphate 3-phosphatase. It contains a tensin like domain as well as a catalytic domain similar to that of the dual specificity protein tyrosine phosphatases. Unlike most of the protein tyrosine phosphatases, this protein preferentially dephosphorylates phosphoinositide substrates. It negatively regulates intracellular levels of phosphatidylinositol-3,4,5-trisphosphate in cells and functions as a tumor suppressor by negatively regulating AKT/PKB signaling pathway. The expression level or the mutation status can be evaluated using primers or oligonucleotides derived from PTEN mRNA sequence found at GenBank Accession No. NM_{—}000314.4 GI:110224474, hereby incorporated by reference and shown here as SEQ ID NO:5:

| | |
|---|---|
| 1 | cctcccctcg cccggcgcgg tcccgtccgc ctctcgctcg cctcccgcct cccctcggtc |
| 61 | ttccgaggcg cccgggctcc cggcgcggcg gcggaggggg cgggcaggcc ggcgggcggt |
| 121 | gatgtggcgg gactctttat gcgctgcggc aggatacgcg ctcggcgctg ggacgcgact |
| 181 | gcgctcagtt ctctcctctc ggaagctgca gccatgatgg aagtttgaga gttgagccgc |
| 241 | tgtgaggcga ggccgggctc aggcgaggga gatgagagac ggcggcggcc gcggcccgga |
| 301 | gcccctctca gcgcctgtga gcagccgcgg gggcagcgcc ctcggggagc cggccggcct |
| 361 | gcggcggcgg cagcggcggc gtttctcgcc tcctcttcgt cttttctaac cgtgcagcct |
| 421 | cttcctcggc ttctcctgaa agggaaggtg gaagccgtgg gctcgggcgg gagccggctg |
| 481 | aggcgcggcg gcggcggcgg cacctcccgc tcctggagcg ggggggagaa gcggcggcgg |
| 541 | cggcggccgc ggcggctgca gctccaggga gggggtctga gtcgcctgtc accatttcca |
| 601 | gggctgggaa cgccggagag ttggtctctc cccttctact gcctccaaca cggcggcggc |
| 661 | ggcggcggca catccaggga cccgggccgg ttttaaacct cccgtccgcc gccgccgcac |
| 721 | cccccgtggc ccgggctccg gaggccgccg gcggaggcag ccgttcggag gattattcgt |
| 781 | cttctcccca ttccgctgcc gccgctgcca ggcctctggc tgctgaggag aagcaggccc |
| 841 | agtcgctgca accatccagc agccgccgca gcagccatta cccggctgcg gtccagagcc |
| 901 | aagcggcggc agagcgaggg gcatcagcta ccgccaagtc cagagccatt tccatcctgc |
| 961 | agaagaagcc ccgccaccag cagcttctgc catctctctc ctcctttttc ttcagccaca |
| 1021 | ggctcccaga catgacagcc atcatcaaag agatcgttag cagaaacaaa aggagatatc |
| 1081 | aagaggatgg attcgactta gacttgacct atatttatcc aaacattatt gctatgggat |
| 1141 | ttcctgcaga aagacttgaa ggcgtataca ggaacaatat tgatgatgta gtaaggtttt |
| 1201 | tggattcaaa gcataaaaac cattacaaga tatacaatct ttgtgctgaa agacattatg |
| 1261 | acaccgccaa atttaattgc agagttgcac aatatccttt tgaagaccat aacccaccac |
| 1321 | agctagaact tatcaaaccc ttttgtgaag atcttgacca atggctaagt gaagatgaca |
| 1381 | atcatgttgc agcaattcac tgtaaagctg gaaagggacg aactggtgta atgatatgtg |
| 1441 | catatttatt acatcggggc aaatttttaa aggcacaaga ggccctagat ttctatgggg |
| 1501 | aagtaaggac cagagacaaa aagggagtaa ctattcccag tcagaggcgc tatgtgtatt |
| 1561 | attatagcta cctgttaaag aatcatctgg attatagacc agtggcactg ttgtttcaca |
| 1621 | agatgatgtt tgaaactatt ccaatgttca gtggcggaac ttgcaatcct cagtttgtgg |
| 1681 | tctgccagct aaaggtgaag atatattcct ccaattcagg acccacacga cgggaagaca |
| 1741 | agttcatgta ctttgagttc cctcagccgt tacctgtgtg tggtgatatc aaagtagagt |
| 1801 | tcttccacaa acagaacaag atgctaaaaa aggacaaaat gtttcacttt tgggtaaata |
| 1861 | cattcttcat accaggacca gaggaaacct cagaaaaagt agaaaatgga agtctatgtg |
| 1921 | atcaagaaat cgatagcatt tgcagtatag agcgtgcaga taatgacaag gaatatctag |
| 1981 | tacttacttt aacaaaaaat gatcttgaca aagcaaataa agacaaagcc aaccgatact |
| 2041 | tttctccaaa ttttaaggtg aagctgtact tcacaaaaac agtagaggag ccgtcaaatc |
| 2101 | cagaggctag cagttcaact tctgtaacac cagatgttag tgacaatgaa cctgatcatt |
| 2161 | atagatattc tgacaccact gactctgatc cagagaatga accttttgat gaagatcagc |
| 2221 | atacacaaat tacaaaagtc tgaatttttt tttatcaaga gggataaaac accatgaaaa |
| 2281 | taaacttgaa taaactgaaa atggaccttt ttttttttaa tggcaatagg acattgtgtc |
| 2341 | agattaccag ttataggaac aattctcttt tcctgaccaa tcttgtttta ccctatacat |
| 2401 | ccacagggtt ttgacacttg ttgtccagtt gaaaaaaggt tgtgtagctg tgtcatgtat |
| 2461 | ataccttttt gtgtcaaaag gacatttaaa attcaattag gattaataaa gatggcactt |
| 2521 | tcccgtttta ttccagtttt ataaaaagtg gagacagact gatgtgtata cgtaggaatt |
| 2581 | ttttcctttt gtgttctgtc accaactgaa gtggctaaag agctttgtga tatactggtt |
| 2641 | cacatcctac ccctttgcac ttgtggcaac agataagttt gcagttggct aagagaggtt |
| 2701 | tccgaagggt tttgctacat tctaatgcat gtattcgggt taggggaatg gagggaatgc |
| 2761 | tcagaaagga aataatttta tgctggactc tggaccatat accatctcca gctatttaca |
| 2821 | cacacctttc tttagcatgc tacagttatt aatctggaca ttcgaggaat tggccgctgt |
| 2881 | cactgcttgt tgtttgcgca ttttttttta aagcatattg gtgctagaaa aggcagctaa |
| 2941 | aggaagtgaa tctgtattgg ggtacaggaa tgaaccttct gcaacatctt aagatccaca |
| 3001 | aatgaaggga tataaaaata atgtcatagg taagaaacac agcaacaatg acttaaccat |
| 3061 | ataaatgtgg aggctatcaa caaagaatgg gcttgaaaca ttataaaaat tgacaatgat |
| 3121 | ttattaaata tgttttctca attgtaacga cttctccatc tcctgtgtaa tcaaggccag |
| 3181 | tgctaaaatt cagatgctgt tagtacctac atcagtcaac aacttacact tattttacta |
| 3241 | gttttcaatc ataatacctg ctgtggatgc ttcatgtgct gcctgcaagc ttcttttttc |
| 3301 | tcattaaata taaaatattt tgtaatgctg cacagaaatt ttcaatttga gattctacag |
| 3361 | taagcgtttt ttttctttga agatttatga tgcacttatt caatagctgt cagccgttcc |
| 3421 | acccttttga ccttacacat tctattacaa tgaattttgc agttttgcac attttttaaa |
| 3481 | tgtcattaac tgttagggaa ttttacttga atactgaata catataatgt ttatattaaa |
| 3541 | aaggacattt gtgttaaaaa ggaaattaga gttgcagtaa actttcaatg ctgcacacaa |
| 3601 | aaaaaagaca tttgattttt cagtagaaat tgtcctacat gtgctttatt gatttgctat |
| 3661 | tgaaagaata gggttttttt tttttttttt tttttttttt ttaaatgtgc agtgttgaat |
| 3721 | catttcttca tagtgctccc ccgagttggg actagggctt caatttcact tcttaaaaaa |
| 3781 | aatcatcata tatttgatat gcccagactg catacgattt taagcggagt acaactacta |
| 3841 | ttgtaaagct aatgtgaaga tattattaaa aaggtttttt tttccagaaa tttggtgtct |
| 3901 | tcaaattata ccttcacctt gacatttgaa tatccagcca ttttgtttct taatggtata |
| 3961 | aaattccatt ttcaataact tattggtgct gaaattgttc actagctgtg gtctgaccta |
| 4021 | gttaatttac aaatacagat tgaataggac ctactagagc agcatttata gagtttgatg |
| 4081 | gcaaatagat taggcagaac ttcatctaaa atattcttag taaataatgt tgacacgttt |
| 4141 | tccatacctt gtcagtttca ttcaacaatt tttaaatttt taacaaagct cttaggattt |
| 4201 | acacatttat atttaaacat tgatatatag agtattgatt gattgctcat aagttaaatt |
| 4261 | ggtaaagtta gagacaacta ttctaacacc tcaccattga aatttatatg ccaccttgtc |
| 4321 | tttcataaaa gctgaaaatt gttacctaaa atgaaaatca acttcatgtt ttgaagatag |
| 4381 | ttataaatat tgttctttgt tacaatttcg ggcaccgcat attaaaacgt aactttattg |
| 4441 | ttccaatatg taacatggag ggccaggtca taaataatga cattataatg ggcttttgca |
| 4501 | ctgttattat ttttcctttg gaatgtgaag gtctgaatga gggttttgat tttgaatgtt |
| 4561 | tcaatgtttt tgagaagcct tgcttacatt ttatggtgta gtcattggaa atggaaaaat |
| 4621 | ggcattatat atattatata tataaatata tattatacat actctcctta ctttatttca |
| 4681 | gttaccatcc ccatagaatt tgacaagaat tgctatgact gaaaggtttt cgagtcctaa |
| 4741 | ttaaaacttt atttatggca gtattcataa ttagcctgaa atgcattctg taggtaatct |
| 4801 | ctgagtttct ggaatatttt cttagacttt ttggatgtgc agcagcttac atgtctgaag |
| 4861 | ttacttgaag gcatcacttt taagaaagct tacagttggg ccctgtacca tcccaagtcc |
| 4921 | tttgtagctc ctcttgaaca tgtttgccat acttttaaaa gggtagttga ataaatagca |
| 4981 | tcaccattct ttgctgtggc acaggttata aacttaagtg gagtttaccg gcagcatcaa |
| 5041 | atgtttcagc tttaaaaaat aaaagtaggg tacaagttta atgtttagtt ctagaaattt |
| 5101 | tgtgcaatat gttcataacg atggctgtgg ttgccacaaa gtgcctcgtt tacctttaaa |
| 5161 | tactgttaat gtgtcatgca tgcagatgga aggggtggaa ctgtgcacta aagtgggggc |
| 5221 | tttaactgta gtatttggca gagttgcctt ctacctgcca gttcaaaagt tcaacctgtt |
| 5281 | ttcatataga atatatatac taaaaaattt cagtctgtta aacagcctta ctctgattca |
| 5341 | gcctcttcag atactcttgt gctgtgcagc agtggctctg tgtgtaaatg ctatgcactg |
| 5401 | aggatacaca aaaataccaa tatgatgtgt acaggataat gcctcatccc aatcagatgt |
| 5461 | ccatttgtta ttgtgtttgt taacaaccct ttatctctta gtgttataaa ctccacttaa |
| 5521 | aactgattaa agtctcattc ttgtcaaaaa aaaaaaaaaa aaaaaaaaaa aa |

Mutations in the PTEN protein can also be detected as is known in the art. The protein sequence of PTEN is found at GenBank Accession No. NP_{—}000305.3 GI:73765544, hereby incorporated by reference, and shown here as SEQ ID NO:6:

| | |
|---|---|
| 1 | mtaiikeivs rnkrryqedg fdldltyiyp niiamgfpae rlegvyrnni ddvvrfldsk |
| 61 | hknhykiynl caerhydtak fncrvaqypf edhnppqlel ikpfcedldq wlseddnhva |
| 121 | aihckagkgr tgvmicayll hrgkflkaqe aldfygevrt rdkkgvtips qrryvyyysy |
| 181 | llknhldyrp vallfhkmmf etipmfsggt cnpqfvvcql kvkiyssnsg ptrredkfmy |
| 241 | fefpqplpvc gdikveffhk qnkmlkkdkm fhfwvntffi pgpeetsekv engslcdqei |
| 301 | dsicsierad ndkeylvltl tkndldkank dkanryfspn fkvklyftkt veepsnpeas |
| 361 | sstsvtpdvs dnepdhyrys dttdsdpene pfdedqhtqi tkv |

In another embodiment, a method for identifying a HER2-positive cancer patient suitable for treatment with a 4-anilinoquinazoline kinase inhibitor and an AKT inhibitor, comprising: (a) measuring the expression level of the SASH1 gene in a sample from the patient; and (b) comparing the expression level of said gene from the patient with the expression level of the gene in a normal tissue sample or a reference expression level (such as the average expression level of the gene in a cell line panel or a cancer cell or tumor panel, or the like), wherein an increase in the expression level of SASH1 indicates the patient is suitable for treatment with the 4-anilinoquinazoline kinase inhibitor and an AKT inhibitor.

In another embodiment, a method for identifying a HER2-positive cancer patient suitable for treatment with a 4-anilinoquinazoline kinase inhibitor and an AKT inhibitor, comprising: (a) obtaining the sequence of the PIK3CA gene from a sample from a patient; (b) identifying any mutations in the PIK3CA gene in a sample from the patient as compared to the wild-type sequence found in SEQ ID NO: 3, 4, or 11, (c) measuring the expression level of the SASH1 gene in the sample from the patient; and (d) comparing the expression level of said gene from the patient with the expression level of the gene in a normal tissue sample or a reference expression level (such as the average expression level of the gene in a cell line panel or a cancer cell or tumor panel, or the like), wherein both a mutation in the PIK3CA gene and an increase in the expression level of SASH1 indicates the patient is suitable for treatment with the 4-anilinoquinazoline kinase inhibitor.

The expression level of the SASH1 gene is measured by measuring the amount or number of molecules of mRNA or transcript in a cell. The measuring can comprise directly measuring the mRNA or transcript obtained from a cell, or measuring the cDNA obtained from an mRNA preparation thereof. Such methods of extracting the mRNA or transcript from a cell, or preparing the cDNA thereof are well known to those skilled in the art. In other embodiments, the expression level of a gene can be measured by measuring or detecting the amount of protein or polypeptide expressed, such as measuring the amount of antibody that specifically binds to the protein in a dot blot or Western blot. The proteins described in the present invention can be overexpressed and purified or isolated to homogeneity and antibodies raised that specifically bind to each protein. Such methods are well known to those skilled in the art.

The SASH1 protein (also known as KIAA0790, O94885, PEPE1, Q8TAI0, Q9H7R7, and SASH 1) is a poorly described protein so named because of SAM and SH3 domains contained in the protein [GenBank Accession Number NM_{—}015278.3 GI:45935384, GenBank mRNA accession number CCDS5212.1, and protein sequence O94885.3 GI:145559526]. SASH1 has been implicated as a putative tumor suppressor gene in both breast and lung cancer. It is thought to be involved in signal transduction, and recent evidence suggests it may be involved in PI3K pathway signaling. The expression level of a gene encoding SASH1 can be measured using an oligonucleotide derived from the nucleotide sequence of GenBank Accession No. NM_{—}015278.3 GI:45935384, hereby incorporated by reference, and shown here as SEQ ID NO: 7:

| | |
|---|---|
| 1 | attttgaaga gaggggtccc ggggagctcc ctccaagatc tagaggctcc gcggccaccc |
| 61 | ctgccgggtc ctgccaagac ttgctagaag gaacgagtcg cgtgccttag ttagttggtt |
| 121 | cccgtcacag gaagaaacgc ctttgcagtg ggtttaattg cttctgggcc gagcgaattc |
| 181 | cccgccgtac aactcagtgg tgcggacttt gcctcctgct accctgttgc tgcgccgagc |
| 241 | ggggtgggaa agtttctgga gttgtcagtc gcgcagcccg tggccaccta gacccgaggt |
| 301 | gcgggcgcct gcgaagggcc cccgcggggt ggccggggcc gccggggcat gcagcgcggg |
| 361 | ggcgcggctc ggtgacgccg cgggcgggga cccggcatcc gggcaggctg cgcgcgggtg |
| 421 | cggggcgagg gcgccgcggg gactgggacg cacggcccgc gcgcgggaca cggccatgga |
| 481 | ggacgcggga gcagctggcc cggggccgga gcctgagccc gagcccgagc cggagcccga |
| 541 | gcccgcgccg gagccggaac cggagcccaa gccgggtgct ggcacatccg aggcgttctc |
| 601 | ccgactctgg accgacgtga tgggtatcct ggacggttca ctgggaaaca tcgatgacct |
| 661 | ggcgcagcag tatgcagatt attacaacac ctgtttctcc gacgtgtgcg agaggatgga |
| 721 | ggagctgcgg aaacggcggg tttcccagga cctggaagtg gagaaacccg atgctagccc |
| 781 | cacgtcactt cagctgcggt cccagatcga agagtcgctt ggcttctgta gcgccgtgtc |
| 841 | aaccccagaa gtggaaagaa agaaccctct tcataaatca aactcagaag acagctctgt |
| 901 | aggaaaagga gactggaaga agaaaaataa gtatttctgg cagaacttcc gaaagaacca |
| 961 | gaaaggaata atgagacaga cttcaaaagg agaagacgtt ggttatgttg ccagtgaaat |
| 1021 | aacgatgagc gatgaggagc ggattcagct aatgatgatg gtcaaagaaa agatgatcac |
| 1081 | aattgaggaa gcacttgcta ggctcaagga atacgaggcc cagcaccggc agtcggctgc |
| 1141 | cctggaccct gctgactggc cagatggttc ttacccaacg tttgatggct catcaaactg |
| 1201 | caattcaaga gaacaatcgg atgatgagac tgaggagtcg gtgaagttta agaggttaca |
| 1261 | caagctggta aactccactc gcagagtcag aaagaaacta attagggtgg aagaaatgaa |
| 1321 | aaaacccagc actgaaggtg gggaggagca cgtgtttgag aattcgccgg tcctggatga |
| 1381 | acggtccgcc ctctactctg gcgtgcacaa gaagcccctt ttctttgatg gctctcctga |
| 1441 | gaaacctccc gaagatgact cagactctct caccacgtct ccatcctcca gcagcctgga |
| 1501 | cacctggggg gctggccgga agttggtcaa aaccttcagc aaaggagaga gccggggcct |
| 1561 | gattaagccc cccaagaaga tggggacatt cttctcctac ccagaagaag aaaaggccca |
| 1621 | gaaagtgtcc cgctccctca ccgaggggga gatgaagaag ggtctcgggt ccctaagcca |
| 1681 | cgggagaacc tgcagttttg gaggatttga cttgacgaat cgctctctgc acgttggcag |
| 1741 | taataattct gacccaatgg gtaaagaagg agactttgtg tacaaagaag tcatcaaatc |
| 1801 | acctactgcc tctcgcatct ctcttgggaa aaaggtgaaa tcagtgaaag agacgatgag |
| 1861 | aaagagaatg tctaaaaaat acagcagctc tgtctctgag caggactcgg gccttgatgg |
| 1921 | aatgcctggc tcccctccgc cttcacagcc cgaccccgaa cacttggaca agcccaagct |
| 1981 | caaggccggg ggttctgtag aaagtcttcg cagttctctc agtgggcaga gctccatgag |
| 2041 | cggtcaaaca gtgagcacca ctgattcctc aaccagcaac cgggaaagcg tcaagtcgga |
| 2101 | agatggggat gacgaagagc cgccttaccg aggcccgttc tgcgggcgtg ccagggtgca |
| 2161 | caccgacttc acccccagtc cctatgacac agactcactc aagctcaaga aaggagatat |
| 2221 | catcgatata atcagcaagc cacccatggg gacctggatg ggcctgctga acaacaaagt |
| 2281 | cggcacgttc aagttcatct acgtggacgt gctcagtgaa gacgaggaga aacccaaacg |
| 2341 | ccccaccagg aggcgtcgga aaggacgacc accccagccc aagtctgtgg aggatctcct |
| 2401 | ggatcggatt aacctaaaag agcacatgcc cactttcctg ttcaatggat atgaagattt |
| 2461 | ggacaccttt aagctgctgg aggaggaaga cttggatgag ttaaatatca gggacccgga |
| 2521 | acacagagct gttctcttga cagcagtgga gctgttacaa gagtatgaca gtaacagcga |
| 2581 | ccagtcagga tcccaggaga agctgctcgt tgacagccag ggcctgagtg gatgctcacc |
| 2641 | ccgagactca ggatgctacg aaagcagtga gaacctggaa aacggcaaga ctcggaaagc |
| 2701 | tagcctccta tctgccaagt catccaccga gcccagcttg aagtctttta gcagaaacca |
| 2761 | gttgggcaat tacccaacat tgcctttaat gaaatcaggg gatgcactga agcagggaca |
| 2821 | ggaggagggc aggctgggtg gtggccttgc cccagacacg tccaagagct gtgacccacc |
| 2881 | tggtgtgact ggtttgaata aaaaccgaag aagcctccca gtttccatct gccggagctg |
| 2941 | tgagaccctg gagggccccc agactgtgga cacttggccc cgatcccatt ccctggatga |
| 3001 | ccttcaagtg gagcctggtg ctgagcaaga cgtgcctacc gaggtgacag aaccgccccc |
| 3061 | tcagattgta cctgaagtgc cacagaagac gaccgcctct tccacgaagg cccagcccct |
| 3121 | ggagcaagac tctgctgtcg acaatgcatt gctactgacc caaagcaaga gattttctga |
| 3181 | acctcagaaa ttgacaacta agaaactgga gggctcaatc gcagcctctg gtcgcggcct |
| 3241 | gtcaccccct cagtgtttgc ccagaaacta tgatgctcag cctcctggag ctaaacacgg |
| 3301 | tttagcaagg acgcctctgg agggccacag aaaaggacac gagtttgaag gaacacacca |
| 3361 | tcccctgggc accaaagaag gggtagatgc tgagcagaga atgcagccca aaattccatc |
| 3421 | acagcctcca cctgttcctg ccaaaaagag cagagaacgc cttgctaacg gactccaccc |
| 3481 | tgttcccatg ggccccagtg gggccctccc cagtcccgat gcgccatgcc tgccagtgaa |
| 3541 | aaggggcagc cccgccagcc ccaccagccc tagcgactgt cccccagcac tggctcccag |
| 3601 | gcctctctca gggcaggcgc ctggcagccc accaagcaca aggccgcccc cctggctctc |
| 3661 | agagctcccc gagaacacaa gcctccagga gcacggtgtg aagctgggcc cggctttgac |
| 3721 | caggaaggtc tcctgtgccc ggggagtgga tctagaaacg ctcactgaaa acaagctgca |
| 3781 | cgctgaaggc atcgatctca cggaggagcc gtattctgat aagcatggcc gctgtgggat |
| 3841 | tcctgaagcc ctggtgcaga gatacgcaga ggacttggat cagcccgagc gggacgtcgc |
| 3901 | cgccaacatg gaccagatcc gggtgaagca gcttcggaag cagcaccgca tggcgattcc |
| 3961 | aagtggtgga ctcacggaaa tctgccgaaa gcccgtctct cctgggtgca tttcgtctgt |
| 4021 | gtcagattgg ctcatttcca tcggtctgcc catgtacgcc ggcaccctct ccaccgcggg |
| 4081 | cttcagcaca ctgagccaag tgccttctct gtctcacact tgccttcagg aggccggcat |
| 4141 | cacagaggag agacacataa gaaagctcct atctgcagcc agactcttca aactgccgcc |
| 4201 | aggccctgag gccatgtagc caggcccgga atgggcctct ctggacaaga gccacccttt |
| 4261 | cactgtgcat atgatgctga tgcaattcct ccatcatctc tggacgtgca gaccagatcc |
| 4321 | agaagaaagg cctggcgtgt ggccaaacag cgtgaaacct tggcacagga ctgaggatcc |
| 4381 | tctcctccag aaaagccccc tcgaggaaat aaattagtgc ggttctcttt gacccccaaa |
| 4441 | gacaagacaa gcacttattt ttattttcag aagacaaaag aaccaagatg ccaactggct |
| 4501 | gcgaatgctc tatctccagt ctgtctctgt gtactggtag aggctgggag gagtaggggg |
| 4561 | cagcctgttc catttctgat agtgcccttg ctcttctgtc tgtcatcttg caggatgccc |
| 4621 | gagggccaga tgggcttagc taggccaaag taacagactc aagagttatt gtacattact |
| 4681 | gaccacgctc atttgttcaa aagttagaac atctggctgc accaggaaaa aaaaaaaaaa |
| 4741 | aaagtcctgt tcttctttag ataaacaaga gacattttca taattgcttt ctagcaatca |
| 4801 | gcttttattt gccttaatat aagcttttaa gcagttatct aactagtgtc cacaaccctg |
| 4861 | taaccatact tccacatctt cagcttaggc agacatcgaa cctctctggg atgtttccag |
| 4921 | caaaagtgag cttttctaat cgtctcattg taacatggct tattttgtag aggtattcat |
| 4981 | cagccacaca cttcatgttg gtttttggtt tttaagctaa ctacaaatct agtaaaaagc |
| 5041 | tatctgaaat tcacaaatat catgtgtgtg cgtgcgtgcg tgcgcgtgtg tgtctgtatt |
| 5101 | catagtgact gcttttggtt ttaaccagtt tagtatcgtt actgtgtgga tcgtcgcgct |
| 5161 | gcagtattga cttggaatcc tgaccatgtc catcccaaaa ttcagtcctc agttaacgga |
| 5221 | tcatgtttgc aaaaggtcac tgtgaggctg catatttcag aaagatgtcc ttaataaggg |
| 5281 | aagtcatgta taagatgttt tctaaaagac ttttcagtat tacaactaat actattatta |
| 5341 | tccttctttt tttatttaga taattctttt aatttaaaca aaggttcact atggaaccag |
| 5401 | acaaatctca ttagccatgt gttaagtatt tgctacttta aattgtttta caactgattt |
| 5461 | cagcacattc tatccttttt tttttttgaa atggagtttc gctcttgtca cccaggctgg |
| 5521 | agtgcaatgg cacgatcttg gctcactgca acctcagtct cccaggttca agtgattctc |
| 5581 | ctgccttagc ctcccgagta gctgggatta taggcaccca ccaccacgcc cagctaattt |
| 5641 | ttgtattatt agtagagaca gggtttcacc atgttggcca ggctggtctc aaactcaact |
| 5701 | cctgacctca ggtggtccac ccgcctcagc ctcccaaagt gctgggatta caggtgtgag |
| 5761 | ccaccgcacc tggcctctgt cctcttttag tctagtgtct ggttttctag caaacagtaa |
| 5821 | atttaaacaa gtaaactatt atggtttcca ttgcttacaa aatgattttc ctttacattc |
| 5881 | ttatcatgaa cactatttta agcatcaaat gcaatcatct aaaatataaa ggtcaatcat |
| 5941 | ttataataga aacaccttga ccacaagccc ttgattgaac attttataat atttcatcta |
| 6001 | cttattaaaa caaataattt cccttgggtt ggaggggaag tgatttcata aattaattag |
| 6061 | aaagccatct ttagcatatt gcttatgtct ggatccatgt ttctgaggaa aaagacattc |
| 6121 | tcaggtgatg tatttttttc atgcattagt atgcattttt aaaaaataat gcatgtttct |
| 6181 | ttaataatta attttcatct tctataagat gccatgtgaa gaagttgtgg aaatgtagaa |
| 6241 | taaaaagcta aagctgccaa atttctgttg aactcttaaa aacagctcat gtttgtttgt |
| 6301 | cctctcgggt tgtggcctag cctatttgca atgtaatgaa gctgcagggt tcttgtatag |
| 6361 | ctaaagcgtt caatgcattt cacgtgctgt ggtggatgtg ggtgctgtag acaggcttct |
| 6421 | tctcttcctg ctctcaaaat acctcggctt gacatttgga cagatcctgt cattgtttaa |
| 6481 | gctgagcaaa aaaccacaca aaagttgtgt aagagatgag ataacaaagg agcgagagaa |
| 6541 | atctcatgtg aatttccaag ttttaattcg ttctccatga aggattttca tttcagtgaa |
| 6601 | agtcgcagca gaagagggaa ctttctggag tttttgagaa tgccaaacca catttttatc |
| 6661 | acacttcttt ggaaatcaat gcctttgcat agaaaatcaa attcagggac cacaaagaat |
| 6721 | tttcagtggg aatgtctagt ctgaggggtc tgaggttgtt tttactttat tgtgttgttt |
| 6781 | aaatatttta aaaatatctt tagcgtttgg tctttttttt ttctgtaaac atttaatttg |
| 6841 | gtctgagaaa agctgaatgt ttgggtgtga cgtttgactg aggtggattg gggctgcctg |
| 6901 | tggacattag tgaacaggtg gtaggcttca ggaatatcca gttttaatca gttgcatttg |
| 6961 | gtacagaatt ttgagtaatg gtgaaaattg ttgtctttgg aaagcacaaa agaaacctgg |
| 7021 | aaaggcagtt cggctcaggt agctacacat aacattgtgt atgattttca cttcaaagct |
| 7081 | gtctggaagg aaatgcagtc agctccagct agtactattt atgtacccag ataactaaga |
| 7141 | tattgtttca tggccttgcc ttagtcagag gcccttttct ctgtcctgaa cccccaggta |
| 7201 | tgggtgaaat tggaaattac taatctattg gaaatcagtt cctgacatag taaagtttgc |
| 7261 | tttcataact gcagcaaaaa aggtcaactt gccaagtcac tgctgccatg tgtgtactgt |
| 7321 | attattttca gaaaaaaata taatagtctg agtccaagtt atcttgattt aaaattgata |
| 7381 | gagaaaagaa actgtcgagc aagttatata acaactaaca acattgcact ttctgtatat |
| 7441 | gaaatcaata tttaaataac ttatttttct ccattgctgt tcttaaaaac attgtaagta |
| 7501 | gctgtaatat accagtacca atatgttctt gcaattgctt cagcccaaga aagctgtgta |
| 7561 | ttgttttaaa aattgtaaaa attattgtga tgattcattt agcataaaga gaggtggacg |
| 7621 | gaagggtttt cctatgtatc aaaacttgtc tataattatg tcatctatgt acctagaaaa |
| 7681 | aagtaaataa atttcttcag ttgaatatg |

In some embodiments, SASH1 protein levels can be detected and compared to normal or reference levels. The SASH1 protein sequence is found at protein sequence GenBank Accession No. O94885.3 GI:145559526, hereby incorporated by reference, and shown here as SEQ ID NO:8:

| | |
|---|---|
| 1 | medagaagpg pepepepepe pepapepepe pkpgagtsea fsrlwtdvmg ildgslgnid |
| 61 | dlaqqyadyy ntcfsdvcer meelrkrrvs qdlevekpda sptslqlrsq ieeslgfcsa |
| 121 | vstpeverkn plhksnseds svgkgdwkkk nkyfwqnfrk nqkgimrqts kgedvgyvas |
| 181 | eitmsdeeri qlmmmvkekm itieealarl keyeaqhrqs aaldpadwpd gsyptfdgss |
| 241 | ncnsreqsdd eteesvkfkr lhklvnstrr vrkklirvee mkkpstegge ehvfenspvl |
| 301 | dersalysgv hkkplffdgs pekppeddsd slttspssss ldtwgagrkl vktfskgesr |
| 361 | glikppkkmg tffsypeeek aqkvsrslte gemkkglgsl shgrtcsfgg fdltnrslhv |
| 421 | gsnnsdpmgk egdfvykevi ksptasrisl gkkvksvket mrkrmskkys ssyseqdsgl |
| 481 | dgmpgsppps qpdpehldkp klkaggsves lrsslsgqss msgqtvsttd sstsnresvk |
| 541 | sedgddeepp yrgpfcgrar vhtdftpspy dtdslklkkg diidiiskpp mgtwmgllnn |
| 601 | kvgtfkfiyv dvlsedeekp krptrrrrkg rppqpksved lldrinlkeh mptflfngye |
| 661 | dldtfkllee edldelnird pehravllta vellqeydsn sdqsgsgekl lvdsqglsgc |
| 721 | sprdsgcyes senlengktr kasllsakss tepslksfsr nqlgnyptlp lmksgdalkq |
| 781 | gqeegrlggg lapdtskscd ppgvtglnkn rrslpvsicr scetlegpqt vdtwprshsl |
| 841 | ddlqvepgae qdvptevtep ppqivpevpq kttasstkaq pleqdsavdn allltqskrf |
| 901 | sepqklttkk legsiaasgr glsppqclpr nydaqppgak hglartpleg hrkghefegt |
| 961 | hhplgtkegv daeqrmqpki psqpppvpak ksrerlangl hpvpmgpsga lpspdapclp |
| 1021 | vkrgspaspt spsdcppala prplsgqapg sppstrpppw lselpentsl qehgvklgpa |
| 1081 | ltrkvscarg vdletltenk lhaegidlte epysdkhgrc gipealvqry aedldqperd |
| 1141 | vaanmdqirv kqlrkqhrma ipsgglteic rkpvspgcis svsdwlisig lpmyagtlst |
| 1201 | agfstlsqvp slshtclqea giteerhirk llsaarlfkl ppgpeam |

In some embodiments of the invention, the method further comprises administering a therapeutically effective amount of the 4-anilinoquinazoline kinase inhibitor to the patient. Compounds and formulations of 4-anilinoquinazoline kinase inhibitors suitable for use in the present invention, and the dosages and methods of administration thereof, are taught in U.S. Pat. Nos. 6,391,874; 6,713,485; 6,727,256; 6,828,320; and 7,157,466, and International Patent Application Nos. PCT/EP97/03672, PCT/EP99/00048, and PCT/US01/20706 (which are incorporated in their entireties by reference).

In some embodiments of the invention, the lapatinib is lapatinib ditosylate monohydrate. Lapatinib ditosylate monohydrate is commercially available under the brand name TYKERB® (GlaxoSmithKline; Research Triangle Park, NC). The prescription information of TYKERB® (Full Prescribing Information, revised March 2007, GlaxoSmithKline), which is incorporated in its entirety by reference, teaches one method of administration of the lapatinib to the patient.

The AKT gene is Homo sapiens v-akt murine thymoma viral oncogene homolog 1 (AKT1) on chromosome 14. The genomic sequence for AKT is found at GenBank Accession No. NG 012188.1 GI:237874257, hereby incorporated by reference and identified as SEQ ID NO:12. The serine-threonine protein kinase encoded by the AKT1 gene is catalytically inactive in serum-starved primary and immortalized fibroblasts. AKT1 and the related AKT2 are activated by platelet-derived growth factor. The activation is rapid and specific, and it is abrogated by mutations in the pleckstrin homology domain of AKT 1. It was shown that the activation occurs through phosphatidylinositol 3-kinase. In the developing nervous system AKT is a critical mediator of growth factor-induced neuronal survival. Survival factors can suppress apoptosis in a transcription-independent manner by activating the serine/threonine kinase AKT1, which then phosphorylates and inactivates components of the apoptotic machinery. Multiple alternatively spliced transcript variants have been found for this gene and are collectively referred to here as AKT. Homo sapiens v-akt murine thymoma viral oncogene homolog 1 (AKT1), transcript variant 2, mRNA at GenBank Accession No: NM_{—}001014432.1 GI:62241014, hereby incorporated by reference, is shown here as SEQ ID NO:9:

| | |
|---|---|
| 1 | cggcaggacc gagcgcggca ggcggctggc ccagcgcagc cagcgcggcc cgaaggacgg |
| 61 | gagcaggcgg ccgagcaccg agcgctgggc accgggcacc gagcggcggc ggcacgcgag |
| 121 | gcccggcccc gagcagcgcc cccgcccgcc gcggcctcca gcccggcccc gcccagcgcc |
| 181 | ggcccgcggg gatgcggagc ggcgggcgcc ggaggccgcg gcccggctag gcccgcgctc |
| 241 | gcgcccggac gcggcggccc ggggcttagg gaaggccgag ccagcctggg tcaaagaagt |
| 301 | caaaggggct gcctggagga ggcagcctgt cagctggtgc atcagaggct gtggccaggc |
| 361 | cagctgggct cggggagcgc cagcctgaga ggagcgcgtg agcgtcgcgg gagcctcggg |
| 421 | caccatgagc gacgtggcta ttgtgaagga gggttggctg cacaaacgag gggagtacat |
| 481 | caagacctgg cggccacgct acttcctcct caagaatgat ggcaccttca ttggctacaa |
| 541 | ggagcggccg caggatgtgg accaacgtga ggctcccctc aacaacttct ctgtggcgca |
| 601 | gtgccagctg atgaagacgg agcggccccg gcccaacacc ttcatcatcc gctgcctgca |
| 661 | gtggaccact gtcatcgaac gcaccttcca tgtggagact cctgaggagc gggaggagtg |
| 721 | gacaaccgcc atccagactg tggctgacgg cctcaagaag caggaggagg aggagatgga |
| 781 | cttccggtcg ggctcaccca gtgacaactc aggggctgaa gagatggagg tgtccctggc |
| 841 | caagcccaag caccgcgtga ccatgaacga gtttgagtac ctgaagctgc tgggcaaggg |
| 901 | cactttcggc aaggtgatcc tggtgaagga gaaggccaca ggccgctact acgccatgaa |
| 961 | gatcctcaag aaggaagtca tcgtggccaa ggacgaggtg gcccacacac tcaccgagaa |
| 1021 | ccgcgtcctg cagaactcca ggcacccctt cctcacagcc ctgaagtact ctttccagac |
| 1081 | ccacgaccgc ctctgctttg tcatggagta cgccaacggg ggcgagctgt tcttccacct |
| 1141 | gtcccgggag cgtgtgttct ccgaggaccg ggcccgcttc tatggcgctg agattgtgtc |
| 1201 | agccctggac tacctgcact cggagaagaa cgtggtgtac cgggacctca agctggagaa |
| 1261 | cctcatgctg gacaaggacg ggcacattaa gatcacagac ttcgggctgt gcaaggaggg |
| 1321 | gatcaaggac ggtgccacca tgaagacctt ttgcggcaca cctgagtacc tggcccccga |
| 1381 | ggtgctggag gacaatgact acggccgtgc agtggactgg tgggggctgg gcgtggtcat |
| 1441 | gtacgagatg atgtgcggtc gcctgccctt ctacaaccag gaccatgaga agctttttga |
| 1501 | gctcatcctc atggaggaga tccgcttccc gcgcacgctt ggtcccgagg ccaagtcctt |
| 1561 | gctttcaggg ctgctcaaga aggaccccaa gcagaggctt ggcgggggct ccgaggacgc |
| 1621 | caaggagatc atgcagcatc gcttctttgc cggtatcgtg tggcagcacg tgtacgagaa |
| 1681 | gaagctcagc ccacccttca agccccaggt cacgtcggag actgacacca ggtattttga |
| 1741 | tgaggagttc acggcccaga tgatcaccat cacaccacct gaccaagatg acagcatgga |
| 1801 | gtgtgtggac agcgagcgca ggccccactt cccccagttc tcctactcgg ccagcggcac |
| 1861 | ggcctgaggc ggcggtggac tgcgctggac gatagcttgg agggatggag aggcggcctc |
| 1921 | gtgccatgat ctgtatttaa tggtttttat ttctcgggtg catttgagag aagccacgct |
| 1981 | gtcctctcga gcccagatgg aaagacgttt ttgtgctgtg ggcagcaccc tcccccgcag |
| 2041 | cggggtaggg aagaaaacta tcctgcgggt tttaatttat ttcatccagt ttgttctccg |
| 2101 | ggtgtggcct cagccctcag aacaatccga ttcacgtagg gaaatgttaa ggacttctgc |
| 2161 | agctatgcgc aatgtggcat tggggggccg ggcaggtcct gcccatgtgt cccctcactc |
| 2221 | tgtcagccag ccgccctggg ctgtctgtca ccagctatct gtcatctctc tggggccctg |
| 2281 | ggcctcagtt caacctggtg gcaccagatg caacctcact atggtatgct ggccagcacc |
| 2341 | ctctcctggg ggtggcaggc acacagcagc cccccagcac taaggccgtg tctctgagga |
| 2401 | cgtcatcgga ggctgggccc ctgggatggg accagggatg ggggatgggc cagggtttac |
| 2461 | ccagtgggac agaggagcaa ggtttaaatt tgttattgtg tattatgttg ttcaaatgca |
| 2521 | ttttgggggt ttttaatctt tgtgacagga aagccctccc ccttcccctt ctgtgtcaca |
| 2581 | gttcttggtg actgtcccac cgggagcctc cccctcagat gatctctcca cggtagcact |
| 2641 | tgaccttttc gacgcttaac ctttccgctg tcgccccagg ccctccctga ctccctgtgg |
| 2701 | gggtggccat ccctgggccc ctccacgcct cctggccaga cgctgccgct gccgctgcac |
| 2761 | cacggcgttt ttttacaaca ttcaacttta gtatttttac tattataata taatatggaa |
| 2821 | ccttccctcc aaattcttca ataaaagttg cttttcaaaa aaaaaaaaaa aaaaaaaa |

The AKT protein found as GenBank Accession No. NP_{—}001014432.1 GI:62241015, and hereby incorporated by reference is shown here at SEQ ID NO:10:

| | |
|---|---|
| 1 | msdvaivkeg wlhkrgeyik twrpryfllk ndgtfigyke rpqdvdqrea plnnfsvaqc |
| 61 | qlmkterprp ntfiirclqw ttviertfhv etpeereewt taiqtvadgl kkqeeeemdf |
| 121 | rsgspsdnsg aeemevslak pkhrvtmnef eylkllgkgt fgkvilvkek atgryyamki |
| 181 | lkkevivakd evahtltenr vlqnsrhpfl talkysfqth drlcfvmeya nggelffhls |
| 241 | rervfsedra rfygaeivsa ldylhseknv vyrdlklenl mldkdghiki tdfglckegi |
| 301 | kdgatmktfc gtpeylapev ledndygrav dwwglgvvmy emmcgrlpfy nqdheklfel |
| 361 | ilmeeirfpr tlgpeaksll sgllkkdpkq rlgggsedak eimqhrffag ivwqhvyekk |
| 421 | lsppfkpqvt setdtryfde eftaqmitit ppdqddsmec vdserrphfp qfsysasgta |

It is contemplated that the AKT inhibitor can target AKT in various ways including targeting the AKT gene (SEQ ID NO:12), AKT mRNA (SEQ ID NO: 9) expression or expression levels, or the AKT protein (SEQ ID NO:10) or protein levels,

Small Molecule Inhibitors.

In some embodiments of the invention, the AKT inhibitor is a small molecule kinase inhibitor, more specifically a serine/threonine kinase inhibitor. In one embodiment, compounds and formulations of an AKT kinase inhibitor include small molecule protein kinase inhibitors such as an aminofuruzan. An aminofuruzan suitable for use in the present invention, and the dosages and methods of administration thereof, include GSK690693 and other compounds taught in U.S. Pat. Nos. 7,157,476; 7,348,339; and 7,547,779, and International Patent Application No. PCT/US2004/027182, hereby incorporated by reference. In other embodiments, the AKT kinase inhibitor suitable for use in the present invention, and the dosages and methods of administration thereof, include heterocyclic carboxamide compounds such as GSK2141795 and other compounds taught in WO2008/098104 and WO/2010/093885, both of which are hereby incorporated by reference. These heterocyclic carboxamide compounds are inhibitors of the activity of one or more of the isoforms of the serine/threonine kinase, Akt. Some of the compounds of described are inhibitors of the activity of all three isoforms of the serine/threonine kinase, Akt.

In one example, we used a novel AKT inhibitor developed by GlaxoSmithKline (GSK690693), which is an ATP-competitive, low-nanomolar pan-Akt kinase inhibitor. It is selective for the three Akt isoforms, although it does inhibit other members of the AGC kinase family.

In another example, we used a novel AKT inhibitor developed by GlaxoSmithKline (GSK2141795), which is a heterocyclic carboxamide compound. that is an inhibitor of the activity of one or more of the isoforms of the serine/threonine kinase, Akt.

In other embodiments, the AKT inhibitor can be a therapeutic including but not limited to, antisense or inhibitory oligonucleotides, RNA interference, siRNAs, aptamers, monoclonal antibodies, and small molecules.

Antibodies.

In one embodiment, a method of treatment using a humanized monoclonal AKT antibody to down-regulate AKT. Specific antibodies can be made by general methods known in the art. A preferred method of generating these antibodies is by first synthesizing peptide fragments. These peptide fragments should likely cover the coding gene region. Since synthesized peptides are not always immunogenic by their own, the peptides should be conjugated to a carrier protein before use. Appropriate carrier proteins include but are not limited to Keyhole limpet hemacyanin (KLH). The conjugated peptides should then be mixed with adjuvant and injected into a mammal, preferably a rabbit through intradermal injection, to elicit an immunogenic response. Samples of serum can be collected and tested by ELISA assay to determine the titer of the antibodies and then harvested.

Polyclonal (e.g., anti-AKT) antibodies can be purified by passing the harvested antibodies through an affinity column. Monoclonal antibodies are preferred over polyclonal antibodies and can be generated according to standard methods known in the art of creating an immortal cell line which expresses the antibody.

Nonhuman antibodies are highly immunogenic in human and that limits their therapeutic potential. In order to reduce their immunogenicity, nonhuman antibodies need to be humanized for therapeutic application. Through the years, many researchers have developed different strategies to humanize the nonhuman antibodies. One such example is using “HuMAb-Mouse” technology available from MEDAREX, Inc. and disclosed by van de Winkel, in U.S. Pat. No. 6,111,166 and hereby incorporated by reference in its entirety. “HuMAb-Mouse” is a strain of transgenic mice which harbor the entire human immunoglobin (Ig) loci and thus can be used to produce fully human monoclonal antibodies.

Inhibitor Oligonucleotides and RNA Interference (RNAi).

The approaches to be taken will depend on the detailed characteristics of the genes, but in some embodiments, will begin with strategies to inhibit RNA transcription since they can, in principal, be used to attack over expressed genes independent of their biochemical composition. Work in the past two decades on transcriptional inhibitors focused on oligodeoxynucleotides and ribozymes. These approaches have had some clinical success but delivery issues limited their clinical utility. Recently, however, advances in short interfering RNA (siRNA) technology and biological understanding have accelerated development of anti-gene therapies (Wall, N. R. & Shi, Y. Small RNA: can RNA interference be exploited for therapy? Lancet 362, 1401-3 (2003); Scanlon, K. J. Anti-genes: siRNA, ribozymes and antisense. Curr Pharm Biotechnol 5, 415-20 (2004); Buckingham, S. D., Esmaeili, B., Wood, M. & Sattelle, D. B. RNA interference: from model organisms towards therapy for neural and neuromuscular disorders. Hum Mol Genet. 13 Spec No 2, R275-88 (2004)). Promising therapeutic approaches include siRNAs complexed with cationic liposomes (Liao, Y., et al., Enhanced paclitaxel cytotoxicity and prolonged animal survival rate by a nonviral-mediated systemic delivery of E1A gene in orthotopic xenograft human breast cancer. Cancer Gene Ther 11, 594-602 (2004); Yano, J. et al. Antitumor activity of small interfering RNA/cationic liposome complex in mouse models of cancer. Clin Cancer Res 10, 7721-6 (2004)), virus vector-mediated RNAi (Zhao, N. et al. Knockdown of Mouse Adult beta-Globin Gene Expression in MEL Cells by Retrovirus Vector-Mediated RNA Interference. Mol Biotechnol 28, 195-200 (2004); Sumimoto, H. et al. Gene therapy for human small-cell lung carcinoma by inactivation of Skp-2 with virally mediated RNA interference. Gene Ther (2004)) and nanoparticles adapted for siRNA (Schiffelers, R. M. et al. Cancer siRNA therapy by tumor selective delivery with ligand-targeted sterically stabilized nanoparticle. Nucleic Acids Res 32, e149 (2004)). In one embodiment, siRNAs against the high priority targets complexed with cationic liposomes and small molecule approaches to inhibit the over expressed candidate genes will allow rapid development of this line of attack.

In some embodiments, the expression of AKT is manipulated. In one embodiment, such manipulation can be made using optimized siRNAs. See Hannon, G. J. RNA interference (2002); Plasterk, R. H. in Science 1263-5 (2002); and Elbashir, S. M. et al. in Nature 494-8 (2001). Strong Pearson correlations between target gene amplification/expression levels and pro-apoptotic effects of siRNAs will indicate that copy number/expression levels determine the extent of apoptotic responses to target gene inhibitors. Spearman rank test correlations between amplification detected and the level of induced apoptosis will indicate response to the targeted therapeutics.

High throughput methods can be used to identify AKT inhibitors such as siRNA and/or small molecular inhibitor formulations to deliver AKT (and other) inhibitors efficiently to cultured cells and xenografts. AKT (and other) inhibitory formulations will be preferentially effective against xenografts that are amplified at the target loci and that these will enhance response to 4-anilinoquinazoline kinase inhibitor compounds. Effective formulations using such methods as described herein can be developed for clinical application.

In one embodiment, RNA interference is used to generate small double-stranded RNA (small interference RNA or siRNA) inhibitors to affect the expression of a candidate gene generally through cleaving and destroying its cognate RNA. Small interference RNA (siRNA) is typically 19-22 nt double-stranded RNA. siRNA can be obtained by chemical synthesis or by DNA-vector based RNAi technology. Using DNA vector based siRNA technology, a small DNA insert (about 70 bp) encoding a short hairpin RNA targeting the gene of interest is cloned into a commercially available vector. The insert-containing vector can be transfected into the cell, and expressing the short hairpin RNA. The hairpin RNA is rapidly processed by the cellular machinery into 19-22 nt double stranded RNA (siRNA). In a preferred embodiment, the siRNA is inserted into a suitable RNAi vector because siRNA made synthetically tends to be less stable and not as effective in transfection.

siRNA can be made using methods and algorithms such as those described by Wang L, Mu F Y. (2004) A Web-based Design Center for Vector-based siRNA and siRNA cassette. Bioinformatics. (In press); Khvorova A, Reynolds A, Jayasena S D. (2003) Functional siRNAs and miRNAs exhibit strand bias. Cell. 115(2):209-16; Harborth J, Elbashir S M, Vandenburgh K, Manning a H, Scaringe S A, Weber K, Tuschl T. (2003) Sequence, chemical, and structural variation of small interfering RNAs and short hairpin RNAs and the effect on mammalian gene silencing. Antisense Nucleic Acid Drug Dev. 13(2):83-105; Reynolds A, Leake D, Boese Q, Scaringe S, Marshall W S, Khvorova A. (2004) Rational siRNA design for RNA interference. Nat. Biotechnol. 22(3):326-30 and Ui-Tei K, Naito Y, Takahashi F, Haraguchi T, Ohki-Hamazaki H, Juni A, Ueda R, Saigo K. (2004) Guidelines for the selection of highly effective siRNA sequences for mammalian and chick RNA interference. Nucleic Acids Res. 32(3):936-48, which are hereby incorporated by reference.

Other tools for constructing siRNA sequences are web tools such as the siRNA Target Finder and Construct Builder available from GenScript (http://www.genscript.com), Oligo Design and Analysis Tools from Integrated DNA Technologies (URL: <http://www.idtdna.com/SciTools/SciTools.aspx>), or siDESIGN™ Center from Dharmacon, Inc. (URL: <http://design.dharmacon.com/default.aspx?source=0>). siRNA are suggested to built using the ORF (open reading frame) as the target selecting region, preferably 50-100 nt downstream of the start codon. Because siRNAs function at the mRNA level, not at the protein level, to design an siRNA, the precise target mRNA nucleotide sequence may be required. Due to the degenerate nature of the genetic code and codon bias, it is difficult to accurately predict the correct nucleotide sequence from the peptide sequence. Additionally, since the function of siRNAs is to cleave mRNA sequences, it is important to use the mRNA nucleotide sequence and not the genomic sequence for siRNA design, although as noted in the Examples, the genomic sequence can be successfully used for siRNA design. However, designs using genomic information might inadvertently target introns and as a result the siRNA would not be functional for silencing the corresponding mRNA.

Rational siRNA design should also minimize off-target effects which often arise from partial complementarity of the sense or antisense strands to an unintended target. These effects are known to have a concentration dependence and one way to minimize off-target effects is often by reducing siRNA concentrations. Another way to minimize such off-target effects is to screen the siRNA for target specificity.

In one embodiment, the siRNA can be modified on the 5′-end of the sense strand to present compounds such as fluorescent dyes, chemical groups, or polar groups. Modification at the 5′-end of the antisense strand has been shown to interfere with siRNA silencing activity and therefore this position is not recommended for modification. Modifications at the other three termini have been shown to have minimal to no effect on silencing activity.

It is recommended that primers be designed to bracket one of the siRNA cleavage sites as this will help eliminate possible bias in the data (i.e., one of the primers should be upstream of the cleavage site, the other should be downstream of the cleavage site). Bias may be introduced into the experiment if the PCR amplifies either 5′ or 3′ of a cleavage site, in part because it is difficult to anticipate how long the cleaved mRNA product may persist prior to being degraded. If the amplified region contains the cleavage site, then no amplification can occur if the siRNA has performed its function.

In some embodiments, siRNA is designed based upon the mRNA sequence of AKT, SEQ ID NO: 9.

In another embodiment, antisense oligonucleotides (“oligos”) can be designed to inhibit AKT and other candidate gene function. Antisense oligonucleotides are short single-stranded nucleic acids, which function by selectively hybridizing to their target mRNA, thereby blocking translation. Translation is inhibited by either RNase H nuclease activity at the DNA:RNA duplex, or by inhibiting ribosome progression, thereby inhibiting protein synthesis. This results in discontinued synthesis and subsequent loss of function of the protein for which the target mRNA encodes.

In some embodiments, antisense oligos are phosphorothioated upon synthesis and purification, and are usually 18-22 bases in length. It is contemplated that the AKT gene antisense oligos may have other modifications such as 2′-O-Methyl RNA, methylphosphonates, chimeric oligos, modified bases and many others modifications, including fluorescent oligos.

In some embodiments, active antisense oligos should be compared against control oligos that have the same general chemistry, base composition, and length as the antisense oligo. These can include inverse sequences, scrambled sequences, and sense sequences. The inverse and scrambled are recommended because they have the same base composition, thus same molecular weight and Tm as the active antisense oligonucleotides. Rational antisense oligo design should consider, for example, that the antisense oligos do not anneal to an unintended mRNA or do not contain motifs known to invoke immunostimulatory responses such as four contiguous G residues, palindromes of 6 or more bases and CG motifs.

Antisense oligonucleotides can be used in vitro in most cell types with good results. However, some cell types require the use of transfection reagents to effect efficient transport into cellular interiors. It is recommended that optimization experiments be performed by using differing final oligonucleotide concentrations in the 1-5 μm range with in most cases the addition of transfection reagents. The window of opportunity, i.e., that concentration where you will obtain a reproducible antisense effect, may be quite narrow, where above that range you may experience confusing non-specific, non-antisense effects, and below that range you may not see any results at all. In a preferred embodiment, down regulation of the targeted mRNA (e.g. AKT mRNA SEQ ID NO: 9) will be demonstrated by use of techniques such as northern blot, real-time PCR, cDNA/oligo array or western blot. The same endpoints can be made for in vivo experiments, while also assessing behavioral endpoints.

For cell culture, antisense oligonucleotides should be re-suspended in sterile nuclease-free water (the use of DEPC-treated water is not recommended). Antisense oligonucleotides can be purified, lyophilized, and ready for use upon re-suspension. Upon suspension, antisense oligonucleotide stock solutions may be frozen at −20° C. and stable for several weeks.

Aptamers.

In another embodiment, aptamer sequences which bind to specific RNA or DNA sequences can be made. Aptamer sequences can be isolated through methods such as those disclosed in U.S. Pat. Nos. 5,756,291; 5,843,653; and 7,329,742, which are hereby incorporated by reference.

It is contemplated that the sequences described herein may be varied to result in substantially homologous sequences which retain the same function as the original. As used herein, a polynucleotide or fragment thereof is “substantially homologous” (or “substantially similar”) to another if, when optimally aligned (with appropriate nucleotide insertions or deletions) with the other polynucleotide (or its complementary strand), using an alignment program such as BLASTN (Altschul, S. F., Gish, W., Miller, W., Myers, E. W. & Lipman, D. J. (1990) “Basic local alignment search tool.” J. Mol. Biol. 215:403-410), and there is nucleotide sequence identity in at least about 80%, preferably at least about 90%, and more preferably at least about 95-98% of the nucleotide bases.

Methods of Treatment.

The AKT therapeutics of the present invention, such as the small molecule AKT inhibitor, also can be used to treat or prevent a variety of disorders associated with reduced survival rate, especially as related to cancers. The AKT therapeutics are administered to a patient in an amount sufficient to elicit a therapeutic response in the patient (e.g., reduction of tumor size and growth rate, prolonged survival rate, reduction in concurrent cancer therapeutics administered to patient). An amount adequate to accomplish this is defined as “therapeutically effective dose or amount.”

The AKT inhibitors of the invention can be administered directly to a mammalian subject using any route known in the art, including e.g., by injection (e.g., intravenous, intraperitoneal, subcutaneous, intramuscular, or intradermal), inhalation, transdermal application, rectal administration, or oral administration.

The pharmaceutical compositions of the invention may comprise a pharmaceutically acceptable carrier. Pharmaceutically acceptable carriers are determined in part by the particular composition being administered, as well as by the particular method used to administer the composition. Accordingly, there are a wide variety of suitable formulations of pharmaceutical compositions of the present invention (see, e.g., Remington's Pharmaceutical Sciences, 17th ed., 1989).

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The phrase “pharmaceutically-acceptable” refers to molecular entities and compositions that do not produce an allergic or similar untoward reaction when administered to a human. The preparation of an aqueous composition that contains a protein as an active ingredient is well understood in the art. Typically, such compositions are prepared as injectables, either as liquid solutions or suspensions; solid forms suitable for solution in, or suspension in, liquid prior to injection can also be prepared. The preparation can also be emulsified.

Administration of the AKT inhibitors of the invention can be in any convenient manner, e.g., by injection, intratumoral injection, intravenous and arterial stents (including eluting stents), cather, oral administration, inhalation, transdermal application, or rectal administration. In some cases, the peptides and nucleic acids are formulated with a pharmaceutically acceptable carrier prior to administration. Pharmaceutically acceptable carriers are determined in part by the particular composition being administered (e.g., nucleic acid or polypeptide), as well as by the particular method used to administer the composition. Accordingly, there are a wide variety of suitable formulations of pharmaceutical compositions of the present invention (see, e.g., Remington's Pharmaceutical Sciences, 17th ed., 1989).

The dose administered to a patient, in the context of the present invention should be sufficient to effect a beneficial therapeutic response in the patient over time. The dose will be determined by the efficacy of the particular vector (e.g. peptide or nucleic acid) employed and the condition of the patient, as well as the body weight or surface area of the patient to be treated. The size of the dose also will be determined by the existence, nature, and extent of any adverse side-effects that accompany the administration of a particular peptide or nucleic acid in a particular patient.

In determining the effective amount of the vector to be administered in the treatment or prophylaxis of diseases or disorder associated with the disease, the physician evaluates circulating plasma levels of the polypeptide or nucleic acid, polypeptide or nucleic acid toxicities, progression of the disease (e.g., breast cancer), and the production of antibodies that specifically bind to the peptide. Typically, the dose equivalent of a polypeptide is from about 0.1 to about 50 mg per kg, preferably from about 1 to about 25 mg per kg, most preferably from about 1 to about 20 mg per kg body weight. In general, the dose equivalent of a naked c acid is from about 1 μg to about 100 μg for a typical 70 kilogram patient, and doses of vectors which include a viral particle are calculated to yield an equivalent amount of therapeutic nucleic acid.

For administration, the AKT inhibitor of the present invention can be administered at a rate determined by the LD-50 of the polypeptide or nucleic acid, and the side-effects of the polypeptide or nucleic acid at various concentrations, as applied to the mass and overall health of the patient. Administration can be accomplished via single or divided doses, e.g., doses administered on a regular basis (e.g., daily) for a period of time (e.g., 2, 3, 4, 5, 6, days or 1-3 weeks or more).

In certain circumstances it will be desirable to deliver the pharmaceutical compositions comprising the AKT inhibitor therapies disclosed herein parenterally, intravenously, intramuscularly, or even intraperitoneally as described in U.S. Pat. No. 5,543,158; U.S. Pat. No. 5,641,515 and U.S. Pat. No. 5,399,363. Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions (U.S. Pat. No. 5,466,468). In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be facilitated by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion (see, e.g., Remington's Pharmaceutical Sciences, 15th Edition, pp. 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, and the general safety and purity standards as required by FDA Office of Biologics standards.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The compositions disclosed herein may be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug-release capsules, and the like.

To date, most studies have been performed with siRNA formulated in sterile saline or phosphate buffered saline (PBS) that has ionic character similar to serum. There are minor differences in PBS compositions (with or without calcium, magnesium, etc.) and investigators should select a formulation best suited to the injection route and animal employed for the study. Lyophilized oligonucleotides and standard or siSTABLE siRNAs are readily soluble in aqueous solution and can be resuspended at concentrations as high as 2.0 mM. However, viscosity of the resultant solutions can sometimes affect the handling of such concentrated solutions.

While lipid formulations have been used extensively for cell culture experiments, the attributes for optimal uptake in cell culture do not match those useful in animals. The principle issue is that the cationic nature of the lipids used in cell culture leads to aggregation when used in animals and results in serum clearance and lung accumulation. Polyethylene glycol complexed-liposome formulations are currently under investigation for delivery of siRNA by several academic and industrial investigators, including Dharmacon, but typically require complex formulation knowledge. There are a few reports that cite limited success using lipid-mediated delivery of plasmids or oligonucleotides in animals.

Oligonucleotides can also be administered via bolus or continuous administration using an ALZET mini-pump (DURECT Corporation). Caution should be observed with bolus administration as studies of antisense oligonucleotides demonstrated certain dosing-related toxicities including hind limb paralysis and death when the molecules were given at high doses and rates of bolus administration. Studies with antisense and ribozymes have shown that the molecules distribute in a related manner whether the dosing is through intravenous (IV), subcutaneous (sub-Q), or intraperitoneal (IP) administration. For most published studies, dosing has been conducted by IV bolus administration through the tail vein. Less is known about the other methods of delivery, although they may be suitable for various studies. Any method of administration will require optimization to ensure optimal delivery and animal health.

For bolus injection, dosing can occur once or twice per day. The clearance of oligonucleotides appears to be biphasic and a fairly large amount of the initial dose is cleared from the urine in the first pass. Dosing should be conducted for a fairly long term, with a one to two week course of administration being preferred. This is somewhat dependent on the model being examined, but several metabolic disorder studies in rodents that have been conducted using antisense oligonucleotides have required this course of dosing to demonstrate clear target knockdown and anticipated outcomes.

Liposomes.

In certain embodiments, the inventors contemplate the use of liposomes, nanocapsules, microparticles, microspheres, lipid particles, vesicles, and the like, for the administration of the AKT inhibitory peptides and nucleic acids of the present invention. In particular, the compositions of the present invention may be formulated for delivery either encapsulated in a lipid particle, a liposome, a vesicle, a nanosphere, or a nanoparticle or the like.

The formation and use of liposomes is generally known to those of skill in the art (see for example, Couvreur et al., 1977; Couvreur, 1988; Lasic, 1998; which describes the use of liposomes and nanocapsules in the targeted antibiotic therapy for intracellular bacterial infections and diseases). Recently, liposomes were developed with improved serum stability and circulation half-times (Gabizon & Papahadjopoulos, 1988; Allen and Choun, 1987; U.S. Pat. No. 5,741,516). Further, various methods of liposome and liposome like preparations as potential drug carriers have been reviewed (Takakura, 1998; Chandran et al., 1997; Margalit, 1995; U.S. Pat. No. 5,567,434; U.S. Pat. No. 5,552,157; U.S. Pat. No. 5,565,213; U.S. Pat. No. 5,738,868 and U.S. Pat. No. 5,795,587).

Liposomes are formed from phospholipids that are dispersed in an aqueous medium and spontaneously form multilamellar concentric bilayer vesicles (also termed multilamellar vesicles (MLVs). MLVs generally have diameters of from 25 nm to 4 m. Sonication of MLVs results in the formation of small unilamellar vesicles (SUVs) with diameters in the range of 200 to 500 Å, containing an aqueous solution in the core.

Liposomes bear resemblance to cellular membranes and are contemplated for use in connection with the present invention as carriers for the peptide compositions. They are widely suitable as both water- and lipid-soluble substances can be entrapped, i.e. in the aqueous spaces and within the bilayer itself, respectively. It is possible that the drug-bearing liposomes may even be employed for site-specific delivery of active agents by selectively modifying the liposomal formulation.

Targeting is generally not a limitation in terms of the present invention. However, should specific targeting be desired, methods are available for this to be accomplished. For example, antibodies may be used to bind to the liposome surface and to direct the liposomes and its contents to particular cell types. Carbohydrate determinants (glycoprotein or glycolipid cell-surface components that play a role in cell-cell recognition, interaction and adhesion) may also be used as recognition sites as they have potential in directing liposomes to particular cell types.

Alternatively, the invention provides for pharmaceutically-acceptable nanocapsule formulations of the compositions of the present invention. Nanocapsules can generally entrap compounds in a stable and reproducible way (Henry-Michelland et al., 1987; Quintanar-Guerrero et al., 1998; Douglas et al., 1987). To avoid side effects due to intracellular polymeric overloading, such ultrafine particles (sized around 0.1 m) should be designed using polymers able to be degraded in vivo. Biodegradable polyalkyl-cyanoacrylate nanoparticles that meet these requirements are contemplated for use in the present invention. Such particles may be are easily made, as described (Couvreur et al., 1980; 1988; zur Muhlen et al., 1998; Zambaux et al. 1998; Pinto-Alphandry et al., 1995 and U.S. Pat. No. 5,145,684).

Combination Therapy.

In some embodiments, the AKT inhibitor is administered in combination with a 4-anilinoquinazoline kinase inhibitor for treating a HER2+ cancer, including breast cancer. For example, an inhibitory AKT siRNA may be administered in conjunction with lapatinib.

The AKT inhibitor and the 4-anilinoquinazoline kinase inhibitor may be administered simultaneously or sequentially. For example, the AKT inhibitor may be administered first, followed by lapatinib. Alternatively, the 4-anilinoquinazoline kinase inhibitor may be administered first, followed by the AKT inhibitor. In some cases, the AKT inhibitor and 4-anilinoquinazoline kinase inhibitor are administered in the same formulation. In other cases the AKT inhibitor and the 4-anilinoquinazoline kinase inhibitor are administered in different formulations. When the AKT inhibitor and the 4-anilinoquinazoline kinase inhibitor are administered in different formulations, their administration may be simultaneous or sequential.

The average dose required to inhibit growth by 50% (GI50) for the lapatinib plus AKT combination as found in the Examples was 60 nM (range 18 nM to 120 nM). In 4 out of 11 HER2 amplified cell lines, there was significant synergy as measured by combination index (upper 95% less than 1) at multiple drug concentrations, while one additional line showed significant synergy at a single concentration. Synergy was observed when cells were co-treated (i.e. simultaneously) with Lapatinib and the AKT inhibitor.

Kits.

The present invention further provides kits for use within any of the above diagnostic methods. Such kits typically comprise two or more components necessary for performing a diagnostic assay. Components may be compounds, reagents, containers and/or equipment. For example, one container within a kit may contain lyophilized primers to amplify and sequence PIK3CA. One or more additional containers may enclose elements, such as reagents or buffers, to be used in the assay. Such kits may also, or alternatively, contain a detection reagent as described above that contains a reporter group suitable for direct or indirect detection of antibody binding.

Kits can also be supplied for therapeutic uses. Thus, the subject composition of the present invention may be provided, usually in a lyophilized form, in a container. For example, the 4-anilinoquinazoline kinase inhibitor and AKT inhibitors described herein are included in the kits with instructions for use, and optionally with buffers, stabilizers, biocides, or inert proteins. It may be desirable to include an inert extender or excipient to dilute the active ingredients, where the excipient may be present in from about 1 to 99% weight of the total composition.

### Example 1

Lapatinib is a dual inhibitor of EGFR/HER2. Recent evidence suggests that resistance to HER2 inhibition by lapatinib may be in part due to re-activation of PI3K-AKT signaling mediated by HER3. The purpose of this study was to screen lapatinib in combination with a pan-AKT inhibitor in a panel of HER2 amplified breast cancer cell lines to determine if this dual inhibition had synergistic effects in preventing cell line growth.

We treated cell lines with lapatinib, AKTi, or a combination of the two to determine if there were synergistic interactions between these targeted agents. Of 11 HER2 positive cell lines tested, four showed strong evidence of synergy, while four cell lines showed little or no synergy (and even some evidence of antagonism). The remaining three cell lines showed an intermediate response. Of the four lines with synergy, all were mutant for PIK3CA, while all four non-synergistic lines were wild-type for PIK3CA. From microarray data, we identified two probe sets (representing one gene, SASH1) at a False Discover Rate of less than 1% that showed a significant association between expression and response. SASH1 has previously been implicated as a tumor suppressor gene in breast, although at this point, it is unclear whether it plays any functional role or is simply a marker for synergistic response to lapatinib and AKTi. Our work demonstrates that a combination of lapatinib plus and AKT inhibitor may be beneficial in HER2 positive patients who also have PI3K pathway mutations. Care should be taken in screening patients prior to treatment, as the combination was found to be antagonistic in some cell lines that did not have PIK3CA mutations. SASH1 may be a useful screening tool in identifying such patients.

We used a panel of breast cancer cell lines that represent the heterogeneity seen in breast tumors for drug screening. Fixed numbers of cells were plated in 96 well plates. Using a Biotek liquid handling robot, cells were treated with nine two-fold dilutions of an AKT inhibitor (GSK690693), a second drug (lapatinib), or a combination of the two. Response was measured using CTG assay as % Growth Inhibition. 50% Growth Inhibition (GI50) was calculated. Synergistic interactions between drugs was calculated by standard median-effect method using % inhibition (inhibitory concentration) readings and reported as a combination index using the Synergy module in R.

Of 11 HER2 positive cell lines tested, four showed strong evidence of synergy in more than half of the nine concentrations used. Five cell lines showed little or no synergy (and even some evidence of antagonism). The remaining two cell lines showed an intermediate response. Of the four lines with synergy, all were mutant for PIK3CA, while only one of the five lines that did not show strong synergy was mutant for PIK3CA. Interestingly, this line did show significant synergy at one dose combination, suggesting that it was likely to be more sensitive to lapatinib+AKTi than the other four lines. From microarray data, we identified two probe sets (representing one gene, SASH1) at a False Discover Rate of less than 5% that showed a significant association between expression and response. SASH1 has previously been implicated as a tumor suppressor gene in breast, although at this point, it is unclear whether it plays any functional role or is simply a marker for synergistic response to lapatinib and AKTi.

Our work demonstrates that a combination of lapatinib plus and AKT inhibitor may be beneficial in HER2 positive patients who also have PI3K pathway mutations. Care should be taken in screening patients prior to treatment, as the combination was found to be antagonistic in some cell lines that did not have PIK3CA mutations. SASH1 may be a useful screening tool in identifying such patients.

### Example 2

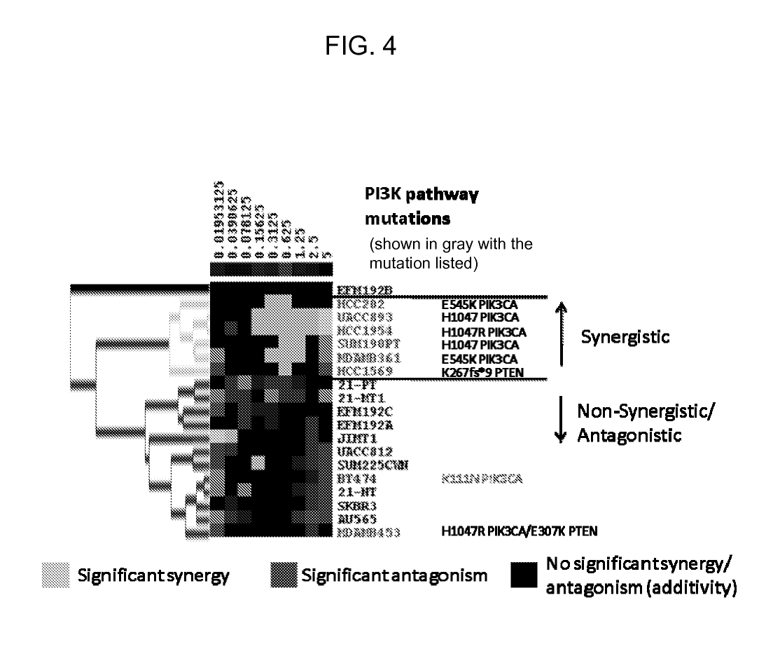

We have increased the number of HER2+ cell lines tested with the combination of Lapatinib+AKT inhibitor (GSK690693) from 11 to 22. The pattern that we observed in Example 1 above, i.e., synergy between these agents was only seen in PI3K mutant lines, has been maintained (see FIG. 4).

### Example 3

We have tested 20/21 of the HER2 amplified cell lines (all but HCC202, which is currently being assessed) with a second AKT inhibitor (GSK2141795) in combination with lapatinib. We were able to calculate synergy values for 19/20 of the cell lines (21-NT cell line failed in the calculation). Cells were treated in the same manner with the same dosages of lapatinib and AKT inhibitor (GSK2141795) as described for the lapatinib plus AKT inhibitor GSK690693 above.

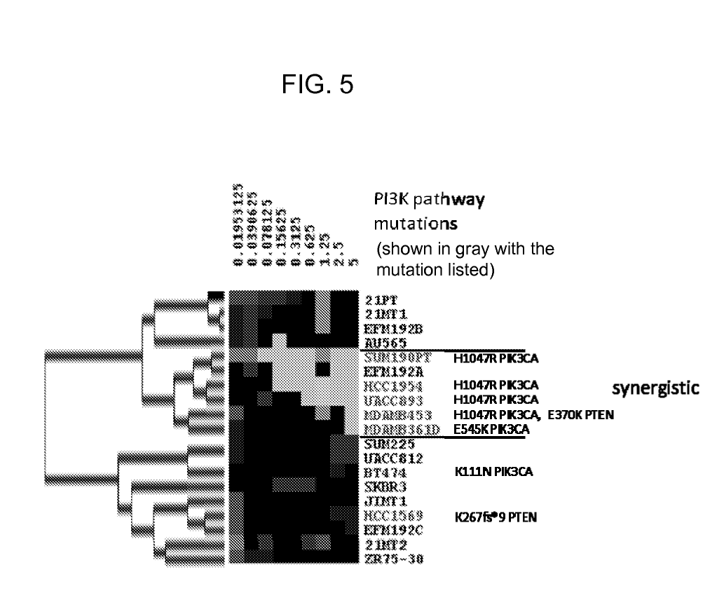

The results of this combination strongly mirror those observed with the AKT inhibitor, as 5/6 of the lines with significant synergy are mutant for PI3K (see FIG. 5). In contrast, of the 13 cell lines that do not show synergy, only 2 have PI3K mutations. Of these, one has a PTEN mutation, which may have a different effect than PIK3CA mutations (the mutation observed in synergistic lines), while the other line (BT474) is the only sample ever described with a K111N mutation in PIK3CA, raising questions about the functional significance of the mutation.

### Example 4

We have taken a non-synergistic cell line (EFM192A) that has wild-type PIK3CA and introduced an H1047R PIK3CA mutant by lentiviral transduction.

The cell line EFM192A was transduced with a mutant H1047R PIK3CA lentiviral vector using standard techniques. Briefly, the lentiviral vector containing mutant PIK3CA was transfected into the cell line 293 along with plasmids containing the viral gag and env genes for packaging. Supernatant containing virus was collected at 48 hours post-transfection and filtered to remove cell debris. Supernatant was then used to transducer EFM192A cells grown to 70% confluence. After 8 hours of treatment of the cells with supernatant, the medium was replaced. Following one day of growth, a selective agent (blasticidin, 10 ug/ml) was added to select for transduced cells. Cells were left in high levels of selection agent for two weeks (by this time, all of the untranduced EFM192A cells treated with blasticidin were killed). The cells were then maintained in lower levels of blasticidin (2-5 ug/ml) to ensure continued presence of the lentiviral delivered gene. Twenty four hours prior to treatment with lapatinib and AKT inhibitor (GSK690693), blasticidin was removed. The transduced cells were treated in the same manner with the same doses of targeted therapeutic agents as described above

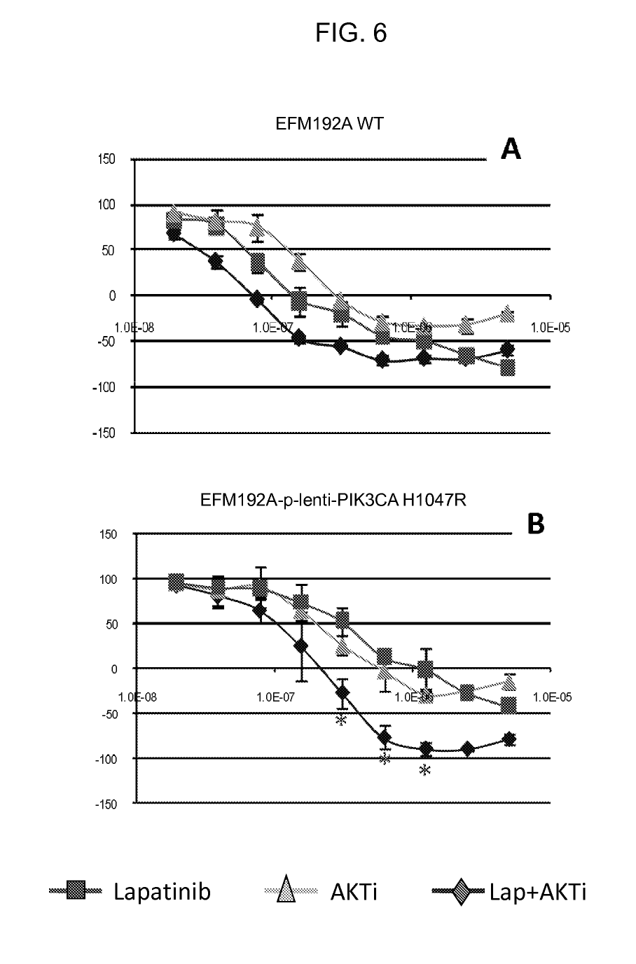

Referring now to FIG. 6, treatment of this altered line with Lapatinb+AKTi (GSK690693) resulted in significant synergy, strongly suggesting that PIK3CA mutations are not only markers of synergy, but have a direct effect on response to the drug combination.

### Example 5

Lapatinib is currently approved for use in patients with advanced HER2 positive breast cancer in conjunction with capecitabine. Patients eligible for Lapatinib+AKT inhibitor therapy would be those HER2 positive patients who also harbor either a PI3K pathway mutation (PIK3CA mutation and possibly PTEN mutation/deletion) or high levels of expression of SASH1. Paraffin embedded tumor blocks could be assessed for mutations in PIK3CA or PTEN using standard sequencing approaches. SASH1 levels could be assessed by RT-PCR approaches from paraffin embedded tissue using primers spanning a short amplicon or by immunohistochemical staining of tissue sections if suitable antibodies can be identified. Patients who have PI3K pathway mutations and/or high levels of SASH1 would then be candidates to receive combination therapy of Lapatinib plus the AKT inhibitor. Lapatinib is an orally bioavailable small molecule inhibitor, while an AKT inhibitor such as GSK690693 or GSK2141795 may be delivered orally or intravenously to the patient.

It is to be understood that the present invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “a gene” includes a plurality of such genes, and so forth.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

The present examples, methods, procedures, specific compounds and molecules are meant to exemplify and illustrate the invention and should in no way be seen as limiting the scope of the invention. Any patents, publications, publicly available sequences mentioned in this specification are indicative of levels of those skilled in the art to which the invention pertains and are hereby incorporated by reference to the same extent as if each was specifically and individually incorporated by reference.

TABLE 1

| | PIK3CA | PTEN | Lap | AKT | L + A |
|---|---|---|---|---|---|
| Cell Line | mutation | mutation | GI50 | GI50 | GI50 |
| HCC202 | p.E545K | nd | 0.068 | 0.015 | 0.018 |
| AU565 | wt | wt | 0.087 | 0.109 | 0.045 |
| MDAMB361 | wt/p.E545K | wt | 0.106 | 0.063 | 0.035 |
| UACC812 | wt | wt | 0.106 | 0.041 | 0.043 |
| MDAMB175 | wt | wt | 0.114 | 0.027 | 0.037 |
| BT474 | wt/p.K111N | wt | 0.116 | 0.089 | 0.050 |
| SKBR3 | wt | wt | 0.163 | 0.054 | 0.046 |
| SUM225 | wt | nd | 0.245 | 0.293 | 0.083 |
| HCC1569 | wt | p.K267fs*9 | 0.420 | 0.122 | 0.083 |
| HCC1954 | p.H1047R | wt | 0.673 | 0.119 | 0.085 |
| SUM190PT | p.H1047R | nd | 0.806 | 0.229 | 0.119 |
| MDAMB453 | p.H1047R | wt/p.E307K | 0.922 | 0.109 | 0.079 |

TABLE 2
List of cell lines used in the study (note: MDAMB175 is not HER2
amplified and so was excluded from the synergy experiments)

| | | | | PIK3CA | PTEN | |
|---|---|---|---|---|---|---|
| Cell Line | Status | ICBP43 | Class | status | status | p53 status |
| 21MT-1 | HER2 amplified | NO | N/A | wt | N/A | N/A |
| 21MT-2 | HER2 amplified | NO | N/A | wt | N/A | N/A |
| 21NT | HER2 amplified | NO | N/A | wt | N/A | N/A |
| 21PT | HER2 amplified | NO | N/A | wt | N/A | N/A |
| AU-565 | HER2 amplified | YES | Luminal | wt | wt | p.R175H |
| BT-474 | HER2 amplified | YES | Luminal | wt/p.K111N | wt | p.E285K |
| EFM192A | HER2 amplified | NO | N/A | wt | N/A | N/A |
| EFM192B | HER2 amplified | NO | N/A | wt | N/A | N/A |
| EFM192C | HER2 amplified | NO | N/A | wt | N/A | N/A |
| HCC1569 | HER2 amplified | YES | BasalA | wt | p.K267fs * 9 | p.E294* |
| HCC1954 | HER2 amplified | YES | BasalA | p.H1047R | wt | wt/p.Y163 |
| HCC202 | HER2 amplified | YES | Luminal | p.E545K | nd | nd |
| JIMT1 | HER2 amplified | NO | N/A | wt | N/A | N/A |
| MDA-MB-175-VII | HER2 over-expressing | YES | Luminal | wt | wt | wt |
| MDA-MB-361 | HER2 amplified | YES | Luminal | wt/p.E545K | wt | wt |
| MDA-MB-453 | HER2 amplified | YES | Luminal | p.H1047R | wt/p.E307K | wt |
| SK-BR-3 | HER2 amplified | YES | Luminal | wt | wt | nd |
| SUM190PT | HER2 amplified | NO | N/A | p.H1047R | nd | nd |
| SUM225CWN | HER2 amplified | NO | Luminal | wt | nd | nd |
| UACC-812 | HER2 amplified | YES | Luminal | wt | wt | wt |
| UACC-893 | HER2 amplified | YES | Luminal | p.H1047R | wt | p.R342* |
| ZR-75-30 | HER2 amplified | YES | Luminal | wt | wt | wt |

We claim:

1. A method for identifying a HER2-positive cancer patient suitable for treatment with a 4-anilinoquinazoline kinase inhibitor and an AKT inhibitor, comprising: (a) obtaining the sequence of the PIK3CA gene, mRNA, or protein from a sample from a patient; and (b) identifying any mutations in the PIK3CA gene in a sample from the patient as compared to the wild-type sequence found in SEQ ID NOS:3, 4, or 11, wherein a mutation in the PIK3CA gene, mRNA or protein sequence indicates the patient is suitable for treatment with the 4-anilinoquinazoline kinase inhibitor and an AKT inhibitor.

2. The method of claim 1, further comprising (c) measuring the expression level of a gene encoding HER2 in a sample from the patient, and (d) comparing said expression level of the gene encoding HER2 in said patient to the expression level of the gene encoding HER2 in a normal tissue sample, a reference expression level, or the average expression level of HER2 in a panel of normal cell lines or cancer cell lines, wherein an increase in the expression level of HER2 indicates the patient is suitable for treatment with the 4-anilinoquinazoline kinase inhibitor.

3. The method of claim 1, wherein the mutation identified in the PIK3 CA gene or mRNA sequence from the patient sample in step (b) expresses a mutant PIK3CA protein with a missense mutation found at amino acid residues 542-545 and/or amino acid residues, 1047-1049.

4. The method of claim 1, wherein the mutation identified in the PIK3 CA protein from the patient sample in step (b) is a missense mutation found at amino acid residues 542-545 and/or amino acid residues, 1047-1049.

5. The method of claim 1, wherein the cancer is breast cancer.

6. The method of claim 1, further comprising administering a therapeutically effective amount of the 4-anilinoquinazoline kinase inhibitor and an AKT inhibitor to the patient.

7. The method of claim 6, wherein the 4-anilinoquinazoline kinase inhibitor is a lapatinib.

8. The method of claim 7, wherein the lapatinib is lapatinib ditosylate monohydrate.

9. The method of claim 8, wherein the AKT inhibitor is an aminofuruzan.

10. The method of claim 9, wherein the AKT inhibitor is GSK690693.

11. The method of claim 6, wherein the AKT inhibitor is a heterocyclic carboxamide.

12. The method of claim 11, wherein the AKT inhibitor is GSK2141795.

13. The method of claim 5, wherein the AKT inhibitor is an antisense or inhibitory oligonucleotide, RNA interference, siRNA, a monoclonal antibody, or a small molecule.

14. The method of claim 2, wherein the HER2 expression level can be detected by measuring the levels of SEQ ID NO: 2 in the patient sample or by immunohistochemical analysis.

15. A method for identifying a HER2-positive cancer patient suitable for treatment with a 4-anilinoquinazoline kinase inhibitor and an AKT inhibitor, comprising: (a) measuring the expression level of the SASH1 gene in a sample from the patient; and (b) comparing the expression level of said gene from the patient with the expression level of the gene in a normal tissue sample or a reference expression level (such as the average expression level of the gene in a cell line panel or a cancer cell or tumor panel, or the like), wherein an increase in the expression level of SASH1 indicates the patient is suitable for treatment with the 4-anilinoquinazoline kinase inhibitor and the AKT inhibitor.

16. The method of claim 15, wherein the cancer is breast cancer.

17. The method of claim 16, further comprising administering a therapeutically effective amount of the 4-anilinoquinazoline kinase inhibitor and an AKT inhibitor to the patient.

18. The method of claim 17, wherein the 4-anilinoquinazoline kinase inhibitor is a lapatinib.

19. The method of claim 18, wherein the lapatinib is lapatinib ditosylate monohydrate.

20. The method of claim 19, wherein the AKT inhibitor is an aminofuruzan.

21. The method of claim 20, wherein the AKT inhibitor is GSK690693.

22. The method of claim 15, wherein the AKT inhibitor is a heterocyclic carboxamide.

23. The method of claim 22, wherein the AKT inhibitor is GSK2141795.

24. A method for identifying a HER2-positive cancer patient suitable for treatment with a 4-anilinoquinazoline kinase inhibitor and an AKT inhibitor, comprising: (a) measuring the expression level of the SASH1 gene in a sample from the patient; and (b) comparing the expression level of said gene from the patient with the expression level of the gene in a normal tissue sample or a reference expression level, wherein an increase in the expression level of SASH1 indicates the patient is suitable for treatment with the 4-anilinoquinazoline kinase inhibitor and an AKT inhibitor.

25. The method of claim 24, wherein the expression level of the SASH1 gene is measured by detecting the levels of SEQ ID NOs: 7 or 8.

26. The method of claim 24, wherein the cancer is breast cancer.

27. The method of claim 24, further comprising administering a therapeutically effective amount of the 4-anilinoquinazoline kinase inhibitor and an AKT inhibitor to the patient.

28. The method of claim 24, wherein the 4-anilinoquinazoline kinase inhibitor is a lapatinib.

29. The method of claim 28, wherein the lapatinib is lapatinib ditosylate monohydrate.

30. The method of claim 24, wherein the AKT inhibitor is an antisense or inhibitory oligonucleotide, RNA interference, siRNA, a monoclonal antibody, or a small molecule.

31. The method of claim 30, wherein the AKT inhibitor is a small molecule.

32. The method of claim 31, wherein the AKT inhibitor is an aminofuruzan.

33. The method of claim 32, wherein the AKT inhibitor is GSK690693.

34. The method of claim 31, wherein the AKT inhibitor is a heterocyclic carboxamide.

35. The method of claim 35, wherein the AKT inhibitor is GSK2141795.
